(19) United States
(12) Patent Application Publication
(10) Pub. No.: US 2005/0147615 A1
(43) Pub. Date: Jul. 7, 2005

(54) Compositions and methods for the therapy and diagnosis of colon cancer

(75) Inventors: Gordon E. King, Shoreline, WA (US); Madeleine Joy Meagher, Seattle, WA (US); Jiangchun Xu, Bellevue, WA (US); Heather Secrist, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Corixa Corporation, Seattle, WA (US)

(21) Appl. No.: 10/961,527

(22) Filed: Oct. 7, 2004

Related U.S. Application Data
(63) Continuation of application No. 09/920,300, filed on Jul. 31, 2001.
(60) Provisional application No. 60/302,051, filed on Jun. 29, 2001.
(60) Provisional application No. 60/279,763, filed on Mar. 28, 2001.
(60) Provisional application No. 60/223,283, filed on Aug. 3, 2000.

(51) Int. Cl. C12Q 1/68; C07H 21/04; A61K 48/00; A61K 39/395; C07K 14/47; C07K 16/30; C12N 5/08

(52) U.S. Cl. 424/155.1; 424/93.21; 435/6; 435/69.1; 435/320.1; 435/372; 530/350; 530/388.8; 536/23.1

(57) ABSTRACT

Compositions and methods for the therapy and diagnosis of cancer, particularly colon cancer, are disclosed. Illustrative compositions comprise one or more colon tumor polypeptides, immunogenic portions thereof, polynucleotides that encode such polypeptides, antigen presenting cell that expresses such polypeptides, and T cells that are specific for cells expressing such polypeptides. The disclosed compositions are useful, for example, in the diagnosis, prevention and/or treatment of diseases, particularly colon cancer.

### STATEMENT REGARDING SEQUENCE LISTING SUBMITTED ON CD-ROM

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 547c4.app.txt which is 1.11 MB and created on Oct. 7, 2004; CD-ROM No. 2 is labeled COPY 2, contains the file 547c4.app.txt which is 1.11 MB and created on Oct. 7, 2004; CD-ROM No. 3 is labeled CRF (Computer Readable Form), contains the file 547c4.app.txt which is 1.11 MB and created on Oct. 7, 2004.

### BACKGROUND OF THE INVENTION

1. Field of the Invention

The present invention relates generally to therapy and diagnosis of cancer, such as colon cancer. The invention is more specifically related to polypeptides, comprising at least a portion of a colon tumor protein, and to polynucleotides encoding such polypeptides. Such polypeptides and polynucleotides are useful in pharmaceutical compositions, e.g., vaccines, and other compositions for the diagnosis and treatment of colon cancer.

2. Description of the Related Art

Cancer is a significant health problem throughout the world. Although advances have been made in detection and therapy of cancer, no vaccine or other universally successful method for prevention and/or treatment is currently available. Current therapies, which are generally based on a combination of chemotherapy or surgery and radiation, continue to prove inadequate in many patients.

Colon cancer is the second most frequently diagnosed malignancy in the United States as well as the second most common cause of cancer death. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death. In spite of considerable research into therapies for the disease, colon cancer remains difficult to diagnose and treat. In spite of considerable research into therapies for these and other cancers, colon cancer remains difficult to diagnose and treat effectively. Accordingly, there is a need in the art for improved methods for detecting and treating such cancers. The present invention fulfills these needs and further provides other related advantages.

In spite of considerable research into therapies for these and other cancers, colon cancer remains difficult to diagnose and treat effectively. Accordingly, there is a need in the art for improved methods for detecting and treating such cancers. The present invention fulfills these needs and further provides other related advantages.

### BRIEF SUMMARY OF THE INVENTION

In one aspect, the present invention provides polynucleotide compositions comprising a sequence selected from the group consisting of:
(a) sequences provided in SEQ ID NO:1-1788;
(b) complements of the sequences provided in SEQ ID NO:1-1788;
(c) sequences consisting of at least 20, 25, 30, 35, 40, 45, 50, 75 and 100 contiguous residues of a sequence provided in SEQ ID NO:1-1788;
(d) sequences that hybridize to a sequence provided in SEQ ID NO:1-1788, under moderate or highly stringent conditions;
(e) sequences having at least 75%, 80%, 85%, 90%, 95%, 96%, 97%, 98% or 99% identity to a sequence of SEQ ID NO:1-1788;
(f) degenerate variants of a sequence provided in SEQ ID NO:1-1788.

In one preferred embodiment, the polynucleotide compositions of the invention are expressed in at least about 20%, more preferably in at least about 30%, and most preferably in at least about 50% of colon tumor samples tested, at a level that is at least about 2-fold, preferably at least about 5-fold, and most preferably at least about 10-fold higher than that for normal tissues.

The present invention, in another aspect, provides polypeptide compositions comprising an amino acid sequence that is encoded by a polynucleotide sequence described above.

The present invention further provides polypeptide compositions comprising an amino acid sequence selected from the group consisting of sequences recited in SEQ ID NO:1789.

In certain preferred embodiments, the polypeptides and/or polynucleotides of the present invention are immunogenic, i.e., they are capable of eliciting an immune response, particularly a humoral and/or cellular immune response, as further described herein.

The present invention further provides fragments, variants and/or derivatives of the disclosed polypeptide and/or polynucleotide sequences, wherein the fragments, variants and/or derivatives preferably have a level of immunogenic activity of at least about 50%, preferably at least about 70% and more preferably at least about 90% of the level of immunogenic activity of a polypeptide sequence set forth in SEQ ID NO:1789 or a polypeptide sequence encoded by a polynucleotide sequence set forth in SEQ ID NO:1-1788.

The present invention further provides polynucleotides that encode a polypeptide described above, expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, the pharmaceutical compositions, e.g., vaccine compositions, are provided for prophylactic or therapeutic applications. Such compositions generally comprise an immunogenic polypeptide or polynucleotide of the invention and an immunostimulant, such as an adjuvant.

The present invention further provides pharmaceutical compositions that comprise: (a) an antibody or antigen-binding fragment thereof that specifically binds to a polypeptide of the present invention, or a fragment thereof; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Illustrative antigen presenting cells include dendritic cells, macrophages, monocytes, fibroblasts and B cells.

Within related aspects, pharmaceutical compositions are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins, typically in the form of pharmaceutical compositions, e.g., vaccine compositions, comprising a physiologically acceptable carrier and/or an immunostimulant. The fusions proteins may comprise multiple immunogenic polypeptides or portions/variants thereof, as described herein, and may further comprise one or more polypeptide segments for facilitating the expression, purification and/or immunogenicity of the polypeptide(s).

Within further aspects, the present invention provides methods for stimulating an immune response in a patient, preferably a T cell response in a human patient, comprising administering a pharmaceutical composition described herein. The patient may be afflicted with colon cancer, in which case the methods provide treatment for the disease, or patient considered at risk for such a disease may be treated prophylactically.

Within further aspects, the present invention provides methods for inhibiting the development of a cancer in a patient, comprising administering to a patient a pharmaceutical composition as recited above. The patient may be afflicted with colon cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with a polypeptide of the present invention, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for a polypeptide of the present invention, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of polypeptide disclosed herein; (ii) a polynucleotide encoding such a polypeptide; and (iii) an antigen-presenting cell that expressed such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer, preferably a colon cancer, in a patient comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody.

The present invention also provides, within other aspects, methods for monitoring the progression of a cancer in a patient. Such methods comprise the steps of:
(a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample, e.g., tumor sample, serum sample, etc., obtained from a patient with an oligonucleotide that hybridizes to a polynucleotide that encodes a polypeptide of the present invention; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes a polypeptide of the present invention; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

| | | |
|---|---|---|
| SEQ ID NO: 1 | is the determined cDNA sequence for clone | ‘58123.1’ |
| SEQ ID NO: 2 | is the determined cDNA sequence for clone | ‘58124.1’ |
| SEQ ID NO: 3 | is the determined cDNA sequence for clone | ‘58125.1’ |
| SEQ ID NO: 4 | is the determined cDNA sequence for clone | ‘58126.1’ |
| SEQ ID NO: 5 | is the determined cDNA sequence for clone | ‘58127.1’ |
| SEQ ID NO: 6 | is the determined cDNA sequence for clone | ‘58128.1’ |
| SEQ ID NO: 7 | is the determined cDNA sequence for clone | ‘58130.1’ |
| SEQ ID NO: 8 | is the determined cDNA sequence for clone | ‘58131.1’ |
| SEQ ID NO: 9 | is the determined cDNA sequence for clone | ‘58132.1’ |
| SEQ ID NO: 10 | is the determined cDNA sequence for clone | ‘58133.1’ |
| SEQ ID NO: 11 | is the determined cDNA sequence for clone | ‘58135.1’ |
| SEQ ID NO: 12 | is the determined cDNA sequence for clone | ‘58136.1’ |
| SEQ ID NO: 13 | is the determined cDNA sequence for clone | ‘58138.1’ |
| SEQ ID NO: 14 | is the determined cDNA sequence for clone | ‘58139.1’ |
| SEQ ID NO: 15 | is the determined cDNA sequence for clone | ‘58141.1’ |
| SEQ ID NO: 16 | is the determined cDNA sequence for clone | ‘58142.1’ |
| SEQ ID NO: 17 | is the determined cDNA sequence for clone | ‘58143.1’ |
| SEQ ID NO: 18 | is the determined cDNA sequence for clone | ‘58144.1’ |
| SEQ ID NO: 19 | is the determined cDNA sequence for clone | ‘58148.1’ |
| SEQ ID NO: 20 | is the determined cDNA sequence for clone | ‘58149.1’ |
| SEQ ID NO: 21 | is the determined cDNA sequence for clone | ‘58150.1’ |
| SEQ ID NO: 22 | is the determined cDNA sequence for clone | ‘58151.1’ |
| SEQ ID NO: 23 | is the determined cDNA sequence for clone | ‘58153.1’ |
| SEQ ID NO: 24 | is the determined cDNA sequence for clone | ‘58154.1’ |
| SEQ ID NO: 25 | is the determined cDNA sequence for clone | ‘58155.1’ |
| SEQ ID NO: 26 | is the determined cDNA sequence for clone | ‘58156.1’ |
| SEQ ID NO: 27 | is the determined cDNA sequence for clone | ‘58159.1’ |
| SEQ ID NO: 28 | is the determined cDNA sequence for clone | ‘58161.1’ |
| SEQ ID NO: 29 | is the determined cDNA sequence for clone | ‘58163.1’ |
| SEQ ID NO: 30 | is the determined cDNA sequence for clone | ‘58164.1’ |
| SEQ ID NO: 31 | is the determined cDNA sequence for clone | ‘58165.1’ |
| SEQ ID NO: 32 | is the determined cDNA sequence for clone | ‘58166.1’ |
| SEQ ID NO: 33 | is the determined cDNA sequence for clone | ‘58167.1’ |
| SEQ ID NO: 34 | is the determined cDNA sequence for clone | ‘58169.1’ |
| SEQ ID NO: 35 | is the determined cDNA sequence for clone | ‘58170.1’ |
| SEQ ID NO: 36 | is the determined cDNA sequence for clone | ‘58171.1’ |
| SEQ ID NO: 37 | is the determined cDNA sequence for clone | ‘58172.1’ |
| SEQ ID NO: 38 | is the determined cDNA sequence for clone | ‘58174.1’ |
| SEQ ID NO: 39 | is the determined cDNA sequence for clone | ‘58176.1’ |
| SEQ ID NO: 40 | is the determined cDNA sequence for clone | ‘58177.1’ |
| SEQ ID NO: 41 | is the determined cDNA sequence for clone | ‘58178.1’ |
| SEQ ID NO: 42 | is the determined cDNA sequence for clone | ‘58183.1’ |
| SEQ ID NO: 43 | is the determined cDNA sequence for clone | ‘58185.1’ |
| SEQ ID NO: 44 | is the determined cDNA sequence for clone | ‘58186.1’ |
| SEQ ID NO: 45 | is the determined cDNA sequence for clone | ‘58189.1’ |
| SEQ ID NO: 46 | is the determined cDNA sequence for clone | ‘58190.1’ |
| SEQ ID NO: 47 | is the determined cDNA sequence for clone | ‘58194.1’ |
| SEQ ID NO: 48 | is the determined cDNA sequence for clone | ‘58196.1’ |
| SEQ ID NO: 49 | is the determined cDNA sequence for clone | ‘58203.1’ |
| SEQ ID NO: 50 | is the determined cDNA sequence for clone | ‘58204.1’ |
| SEQ ID NO: 51 | is the determined cDNA sequence for clone | ‘58205.1’ |
| SEQ ID NO: 52 | is the determined cDNA sequence for clone | ‘58206.1’ |
| SEQ ID NO: 53 | is the determined cDNA sequence for clone | ‘58208.1’ |
| SEQ ID NO: 54 | is the determined cDNA sequence for clone | ‘58214.1’ |
| SEQ ID NO: 55 | is the determined cDNA sequence for clone | ‘58215.1’ |
| SEQ ID NO: 56 | is the determined cDNA sequence for clone | ‘58216.1’ |
| SEQ ID NO: 57 | is the determined cDNA sequence for clone | ‘58218.1’ |
| SEQ ID NO: 58 | is the determined cDNA sequence for clone | ‘69339.1’ |
| SEQ ID NO: 59 | is the determined cDNA sequence for clone | ‘69340.1’ |
| SEQ ID NO: 60 | is the determined cDNA sequence for clone | ‘69341.1’ |
| SEQ ID NO: 61 | is the determined cDNA sequence for clone | ‘69342.1’ |
| SEQ ID NO: 62 | is the determined cDNA sequence for clone | ‘69343.1’ |
| SEQ ID NO: 63 | is the determined cDNA sequence for clone | ‘69344.1’ |
| SEQ ID NO: 64 | is the determined cDNA sequence for clone | ‘69345.1’ |
| SEQ ID NO: 65 | is the determined cDNA sequence for clone | ‘69346.1’ |
| SEQ ID NO: 66 | is the determined cDNA sequence for clone | ‘69347.1’ |
| SEQ ID NO: 67 | is the determined cDNA sequence for clone | ‘69348.1’ |
| SEQ ID NO: 68 | is the determined cDNA sequence for clone | ‘69349.1’ |
| SEQ ID NO: 69 | is the determined cDNA sequence for clone | ‘69350.1’ |
| SEQ ID NO: 70 | is the determined cDNA sequence for clone | ‘69351.1’ |
| SEQ ID NO: 71 | is the determined cDNA sequence for clone | ‘69352.1’ |
| SEQ ID NO: 72 | is the determined cDNA sequence for clone | ‘69353.1’ |
| SEQ ID NO: 73 | is the determined cDNA sequence for clone | ‘69354.1’ |
| SEQ ID NO: 74 | is the determined cDNA sequence for clone | ‘69355.1’ |
| SEQ ID NO: 75 | is the determined cDNA sequence for clone | ‘69357.1’ |
| SEQ ID NO: 76 | is the determined cDNA sequence for clone | ‘69358.1’ |
| SEQ ID NO: 77 | is the determined cDNA sequence for clone | ‘69360.1’ |
| SEQ ID NO: 78 | is the determined cDNA sequence for clone | ‘69965.1’ |
| SEQ ID NO: 79 | is the determined cDNA sequence for clone | ‘69966.1’ |
| SEQ ID NO: 80 | is the determined cDNA sequence for clone | ‘69967.1’ |
| SEQ ID NO: 81 | is the determined cDNA sequence for clone | ‘69969.1’ |
| SEQ ID NO: 82 | is the determined cDNA sequence for clone | ‘69970.1’ |
| SEQ ID NO: 83 | is the determined cDNA sequence for clone | ‘69971.1’ |
| SEQ ID NO: 84 | is the determined cDNA sequence for clone | ‘69972.1’ |
| SEQ ID NO: 85 | is the determined cDNA sequence for clone | ‘69974.1’ |
| SEQ ID NO: 86 | is the determined cDNA sequence for clone | ‘69975.1’ |
| SEQ ID NO: 87 | is the determined cDNA sequence for clone | ‘69976.1’ |
| SEQ ID NO: 88 | is the determined cDNA sequence for clone | ‘69977.1’ |
| SEQ ID NO: 89 | is the determined cDNA sequence for clone | ‘69978.1’ |
| SEQ ID NO: 90 | is the determined cDNA sequence for clone | ‘69980.1’ |
| SEQ ID NO: 91 | is the determined cDNA sequence for clone | ‘69981.1’ |
| SEQ ID NO: 92 | is the determined cDNA sequence for clone | ‘69982.1’ |
| SEQ ID NO: 93 | is the determined cDNA sequence for clone | ‘69983.1’ |
| SEQ ID NO: 94 | is the determined cDNA sequence for clone | ‘69984.1’ |
| SEQ ID NO: 95 | is the determined cDNA sequence for clone | ‘69985.1’ |
| SEQ ID NO: 96 | is the determined cDNA sequence for clone | ‘69986.1’ |
| SEQ ID NO: 97 | is the determined cDNA sequence for clone | ‘69987.1’ |
| SEQ ID NO: 98 | is the determined cDNA sequence for clone | ‘69989.1’ |
| SEQ ID NO: 99 | is the determined cDNA sequence for clone | ‘69990.1’ |
| SEQ ID NO: 100 | is the determined cDNA sequence for clone | ‘69991.1’ |
| SEQ ID NO: 101 | is the determined cDNA sequence for clone | ‘69992.1’ |
| SEQ ID NO: 102 | is the determined cDNA sequence for clone | ‘69993.1’ |
| SEQ ID NO: 103 | is the determined cDNA sequence for clone | ‘69994.1’ |
| SEQ ID NO: 104 | is the determined cDNA sequence for clone | ‘69995.1’ |
| SEQ ID NO: 105 | is the determined cDNA sequence for clone | ‘69996.1’ |
| SEQ ID NO: 106 | is the determined cDNA sequence for clone | ‘69997.1’ |
| SEQ ID NO: 107 | is the determined cDNA sequence for clone | ‘69999.1’ |
| SEQ ID NO: 108 | is the determined cDNA sequence for clone | ‘70000.1’ |
| SEQ ID NO: 109 | is the determined cDNA sequence for clone | ‘70001.1’ |
| SEQ ID NO: 110 | is the determined cDNA sequence for clone | ‘70002.1’ |
| SEQ ID NO: 111 | is the determined cDNA sequence for clone | ‘70003.1’ |
| SEQ ID NO: 112 | is the determined cDNA sequence for clone | ‘70004.1’ |
| SEQ ID NO: 113 | is the determined cDNA sequence for clone | ‘70006.1’ |
| SEQ ID NO: 114 | is the determined cDNA sequence for clone | ‘70007.1’ |
| SEQ ID NO: 115 | is the determined cDNA sequence for clone | ‘70009.1’ |
| SEQ ID NO: 116 | is the determined cDNA sequence for clone | ‘70010.1’ |
| SEQ ID NO: 117 | is the determined cDNA sequence for clone | ‘70011.1’ |
| SEQ ID NO: 118 | is the determined cDNA sequence for clone | ‘70012.1’ |
| SEQ ID NO: 119 | is the determined cDNA sequence for clone | ‘70013.1’ |
| SEQ ID NO: 120 | is the determined cDNA sequence for clone | ‘70015.1’ |
| SEQ ID NO: 121 | is the determined cDNA sequence for clone | ‘70016.1’ |
| SEQ ID NO: 122 | is the determined cDNA sequence for clone | ‘70017.1’ |
| SEQ ID NO: 123 | is the determined cDNA sequence for clone | ‘70018.1’ |
| SEQ ID NO: 124 | is the determined cDNA sequence for clone | ‘70020.1’ |
| SEQ ID NO: 125 | is the determined cDNA sequence for clone | ‘70021.1’ |
| SEQ ID NO: 126 | is the determined cDNA sequence for clone | ‘70022.1’ |
| SEQ ID NO: 127 | is the determined cDNA sequence for clone | ‘70024.1’ |
| SEQ ID NO: 128 | is the determined cDNA sequence for clone | ‘70025.1’ |
| SEQ ID NO: 129 | is the determined cDNA sequence for clone | ‘70026.1’ |
| SEQ ID NO: 130 | is the determined cDNA sequence for clone | ‘70028.1’ |
| SEQ ID NO: 131 | is the determined cDNA sequence for clone | ‘70029.1’ |
| SEQ ID NO: 132 | is the determined cDNA sequence for clone | ‘70030.1’ |
| SEQ ID NO: 133 | is the determined cDNA sequence for clone | ‘70032.1’ |
| SEQ ID NO: 134 | is the determined cDNA sequence for clone | ‘70033.1’ |
| SEQ ID NO: 135 | is the determined cDNA sequence for clone | ‘70034.1’ |
| SEQ ID NO: 136 | is the determined cDNA sequence for clone | ‘70036.1’ |
| SEQ ID NO: 137 | is the determined cDNA sequence for clone | ‘70037.1’ |
| SEQ ID NO: 138 | is the determined cDNA sequence for clone | ‘70038.1’ |
| SEQ ID NO: 139 | is the determined cDNA sequence for clone | ‘70040.1’ |
| SEQ ID NO: 140 | is the determined cDNA sequence for clone | ‘70041.1’ |
| SEQ ID NO: 141 | is the determined cDNA sequence for clone | ‘70044.1’ |
| SEQ ID NO: 142 | is the determined cDNA sequence for clone | ‘70045.1’ |
| SEQ ID NO: 143 | is the determined cDNA sequence for clone | ‘69489.1’ |
| SEQ ID NO: 144 | is the determined cDNA sequence for clone | ‘69490.1’ |
| SEQ ID NO: 145 | is the determined cDNA sequence for clone | ‘69491.1’ |
| SEQ ID NO: 146 | is the determined cDNA sequence for clone | ‘69492.1’ |
| SEQ ID NO: 147 | is the determined cDNA sequence for clone | ‘69493.1’ |
| SEQ ID NO: 148 | is the determined cDNA sequence for clone | ‘69494.1’ |
| SEQ ID NO: 149 | is the determined cDNA sequence for clone | ‘69496.1’ |
| SEQ ID NO: 150 | is the determined cDNA sequence for clone | ‘69497.1’ |
| SEQ ID NO: 151 | is the determined cDNA sequence for clone | ‘69498.1’ |
| SEQ ID NO: 152 | is the determined cDNA sequence for clone | ‘69499.1’ |
| SEQ ID NO: 153 | is the determined cDNA sequence for clone | ‘69500.1’ |
| SEQ ID NO: 154 | is the determined cDNA sequence for clone | ‘69501.1’ |
| SEQ ID NO: 155 | is the determined cDNA sequence for clone | ‘69503.1’ |
| SEQ ID NO: 156 | is the determined cDNA sequence for clone | ‘69505.1’ |
| SEQ ID NO: 157 | is the determined cDNA sequence for clone | ‘69506.1’ |
| SEQ ID NO: 158 | is the determined cDNA sequence for clone | ‘69507.1’ |
| SEQ ID NO: 159 | is the determined cDNA sequence for clone | ‘69508.1’ |
| SEQ ID NO: 160 | is the determined cDNA sequence for clone | ‘69509.1’ |
| SEQ ID NO: 161 | is the determined cDNA sequence for clone | ‘69511.1’ |
| SEQ ID NO: 162 | is the determined cDNA sequence for clone | ‘69512.1’ |
| SEQ ID NO: 163 | is the determined cDNA sequence for clone | ‘69513.1’ |
| SEQ ID NO: 164 | is the determined cDNA sequence for clone | ‘69514.1’ |
| SEQ ID NO: 165 | is the determined cDNA sequence for clone | ‘69516.1’ |
| SEQ ID NO: 166 | is the determined cDNA sequence for clone | ‘69517.1’ |
| SEQ ID NO: 167 | is the determined cDNA sequence for clone | ‘69518.1’ |
| SEQ ID NO: 168 | is the determined cDNA sequence for clone | ‘69520.1’ |
| SEQ ID NO: 169 | is the determined cDNA sequence for clone | ‘69521.1’ |
| SEQ ID NO: 170 | is the determined cDNA sequence for clone | ‘69523.1’ |
| SEQ ID NO: 171 | is the determined cDNA sequence for clone | ‘69524.1’ |
| SEQ ID NO: 172 | is the determined cDNA sequence for clone | ‘69525.1’ |
| SEQ ID NO: 173 | is the determined cDNA sequence for clone | ‘69526.1’ |
| SEQ ID NO: 174 | is the determined cDNA sequence for clone | ‘69527.1’ |
| SEQ ID NO: 175 | is the determined cDNA sequence for clone | ‘69528.1’ |
| SEQ ID NO: 176 | is the determined cDNA sequence for clone | ‘69529.1’ |
| SEQ ID NO: 177 | is the determined cDNA sequence for clone | ‘69530.1’ |
| SEQ ID NO: 178 | is the determined cDNA sequence for clone | ‘70019.1’ |
| SEQ ID NO: 179 | is the determined cDNA sequence for clone | ‘70023.1’ |
| SEQ ID NO: 180 | is the determined cDNA sequence for clone | ‘70035.1’ |
| SEQ ID NO: 181 | is the determined cDNA sequence for clone | ‘70039.1’ |
| SEQ ID NO: 182 | is the determined cDNA sequence for clone | ‘70046.1’ |
| SEQ ID NO: 183 | is the determined cDNA sequence for clone | ‘70047.1’ |
| SEQ ID NO: 184 | is the determined cDNA sequence for clone | ‘70048.1’ |
| SEQ ID NO: 185 | is the determined cDNA sequence for clone | ‘70049.1’ |
| SEQ ID NO: 186 | is the determined cDNA sequence for clone | ‘70050.1’ |
| SEQ ID NO: 187 | is the determined cDNA sequence for clone | ‘70051.1’ |
| SEQ ID NO: 188 | is the determined cDNA sequence for clone | ‘70052.1’ |
| SEQ ID NO: 189 | is the determined cDNA sequence for clone | ‘70053.1’ |
| SEQ ID NO: 190 | is the determined cDNA sequence for clone | ‘70054.1’ |
| SEQ ID NO: 191 | is the determined cDNA sequence for clone | ‘70055.1’ |
| SEQ ID NO: 192 | is the determined cDNA sequence for clone | ‘70058.1’ |
| SEQ ID NO: 193 | is the determined cDNA sequence for clone | ‘70059.1’ |
| SEQ ID NO: 194 | is the determined cDNA sequence for clone | ‘70060.1’ |
| SEQ ID NO: 195 | is the determined cDNA sequence for clone | ‘70061.1’ |
| SEQ ID NO: 196 | is the determined cDNA sequence for clone | ‘70064.1’ |
| SEQ ID NO: 197 | is the determined cDNA sequence for clone | ‘70065.1’ |
| SEQ ID NO: 198 | is the determined cDNA sequence for clone | ‘70066.1’ |
| SEQ ID NO: 199 | is the determined cDNA sequence for clone | ‘70067.1’ |
| SEQ ID NO: 200 | is the determined cDNA sequence for clone | ‘70068.1’ |
| SEQ ID NO: 201 | is the determined cDNA sequence for clone | ‘70069.1’ |
| SEQ ID NO: 202 | is the determined cDNA sequence for clone | ‘70070.1’ |
| SEQ ID NO: 203 | is the determined cDNA sequence for clone | ‘70071.1’ |
| SEQ ID NO: 204 | is the determined cDNA sequence for clone | ‘70072.1’ |
| SEQ ID NO: 205 | is the determined cDNA sequence for clone | ‘70073.1’ |
| SEQ ID NO: 206 | is the determined cDNA sequence for clone | ‘70074.1’ |
| SEQ ID NO: 207 | is the determined cDNA sequence for clone | ‘70075.1’ |
| SEQ ID NO: 208 | is the determined cDNA sequence for clone | ‘70077.1’ |
| SEQ ID NO: 209 | is the determined cDNA sequence for clone | ‘70078.1’ |
| SEQ ID NO: 210 | is the determined cDNA sequence for clone | ‘70079.1’ |
| SEQ ID NO: 211 | is the determined cDNA sequence for clone | ‘70144.1’ |
| SEQ ID NO: 212 | is the determined cDNA sequence for clone | ‘70145.1’ |
| SEQ ID NO: 213 | is the determined cDNA sequence for clone | ‘70146.1’ |
| SEQ ID NO: 214 | is the determined cDNA sequence for clone | ‘70147.1’ |
| SEQ ID NO: 215 | is the determined cDNA sequence for clone | ‘70148.1’ |
| SEQ ID NO: 216 | is the determined cDNA sequence for clone | ‘70149.1’ |
| SEQ ID NO: 217 | is the determined cDNA sequence for clone | ‘70150.1’ |
| SEQ ID NO: 218 | is the determined cDNA sequence for clone | ‘70151.1’ |
| SEQ ID NO: 219 | is the determined cDNA sequence for clone | ‘70152.1’ |
| SEQ ID NO: 220 | is the determined cDNA sequence for clone | ‘70153.1’ |
| SEQ ID NO: 221 | is the determined cDNA sequence for clone | ‘70154.1’ |
| SEQ ID NO: 222 | is the determined cDNA sequence for clone | ‘70155.1’ |
| SEQ ID NO: 223 | is the determined cDNA sequence for clone | ‘70158.1’ |
| SEQ ID NO: 224 | is the determined cDNA sequence for clone | ‘70159.1’ |
| SEQ ID NO: 225 | is the determined cDNA sequence for clone | ‘70160.1’ |
| SEQ ID NO: 226 | is the determined cDNA sequence for clone | ‘70161.1’ |
| SEQ ID NO: 227 | is the determined cDNA sequence for clone | ‘70162.1’ |
| SEQ ID NO: 228 | is the determined cDNA sequence for clone | ‘70163.1’ |
| SEQ ID NO: 229 | is the determined cDNA sequence for clone | ‘70165.1’ |
| SEQ ID NO: 230 | is the determined cDNA sequence for clone | 63690041 R0663: A02 |
| SEQ ID NO: 231 | is the determined cDNA sequence for clone | 63690042 R0663: A03 |
| SEQ ID NO: 232 | is the determined cDNA sequence for clone | 63690043 R0663: A05 |
| SEQ ID NO: 233 | is the determined cDNA sequence for clone | 63690045 R0663: A07 |
| SEQ ID NO: 234 | is the determined cDNA sequence for clone | 63690046 R0663: A08 |
| SEQ ID NO: 235 | is the determined cDNA sequence for clone | 63690047 R0663: A09 |
| SEQ ID NO: 236 | is the determined cDNA sequence for clone | 63690048 R0663: A10 |
| SEQ ID NO: 237 | is the determined cDNA sequence for clone | 63690049 R0663: A11 |
| SEQ ID NO: 238 | is the determined cDNA sequence for clone | 63690050 R0663: A12 |
| SEQ ID NO: 239 | is the determined cDNA sequence for clone | 63690051 R0663: B01 |
| SEQ ID NO: 240 | is the determined cDNA sequence for clone | 63690052 R0663: B02 |
| SEQ ID NO: 241 | is the determined cDNA sequence for clone | 63690053 R0663: B03 |
| SEQ ID NO: 242 | is the determined cDNA sequence for clone | 63690054 R0663: B04 |
| SEQ ID NO: 243 | is the determined cDNA sequence for clone | 63690055 R0663: B05 |
| SEQ ID NO: 244 | is the determined cDNA sequence for clone | 63690056 R0663: B06 |
| SEQ ID NO: 245 | is the determined cDNA sequence for clone | 63690057 R0663: B07 |
| SEQ ID NO: 246 | is the determined cDNA sequence for clone | 63690058 R0663: B08 |
| SEQ ID NO: 247 | is the determined cDNA sequence for clone | 63690059 R0663: B09 |
| SEQ ID NO: 248 | is the determined cDNA sequence for clone | 63690061 R0663: B11 |
| SEQ ID NO: 249 | is the determined cDNA sequence for clone | 63690062 R0663: B12 |
| SEQ ID NO: 250 | is the determined cDNA sequence for clone | 63690063 R0663: C01 |
| SEQ ID NO: 251 | is the determined cDNA sequence for clone | 63690065 R0663: C03 |
| SEQ ID NO: 252 | is the determined cDNA sequence for clone | 63690066 R0663: C04 |
| SEQ ID NO: 253 | is the determined cDNA sequence for clone | 63690067 R0663: C05 |
| SEQ ID NO: 254 | is the determined cDNA sequence for clone | 63690068 R0663: C06 |
| SEQ ID NO: 255 | is the determined cDNA sequence for clone | 63690069 R0663: C07 |
| SEQ ID NO: 256 | is the determined cDNA sequence for clone | 63690070 R0663: C08 |
| SEQ ID NO: 257 | is the determined cDNA sequence for clone | 63690071 R0663: C09 |
| SEQ ID NO: 258 | is the determined cDNA sequence for clone | 63690072 R0663: C10 |
| SEQ ID NO: 259 | is the determined cDNA sequence for clone | 63690073 R0663: C11 |
| SEQ ID NO: 260 | is the determined cDNA sequence for clone | 63690074 R0663: C12 |
| SEQ ID NO: 261 | is the determined cDNA sequence for clone | 63690075 R0663: D01 |
| SEQ ID NO: 262 | is the determined cDNA sequence for clone | 63690077 R0663: D03 |
| SEQ ID NO: 263 | is the determined cDNA sequence for clone | 63690078 R0663: D04 |
| SEQ ID NO: 264 | is the determined cDNA sequence for clone | 63690079 R0663: D05 |
| SEQ ID NO: 265 | is the determined cDNA sequence for clone | 63690080 R0663: D06 |
| SEQ ID NO: 266 | is the determined cDNA sequence for clone | 63690081 R0663: D07 |
| SEQ ID NO: 267 | is the determined cDNA sequence for clone | 63690082 R0663: D08 |
| SEQ ID NO: 268 | is the determined cDNA sequence for clone | 63690083 R0663: D09 |
| SEQ ID NO: 269 | is the determined cDNA sequence for clone | 63690084 R0663: D10 |
| SEQ ID NO: 270 | is the determined cDNA sequence for clone | 63690085 R0663: D11 |
| SEQ ID NO: 271 | is the determined cDNA sequence for clone | 63690086 R0663: D12 |
| SEQ ID NO: 272 | is the determined cDNA sequence for clone | 63690087 R0663: E01 |
| SEQ ID NO: 273 | is the determined cDNA sequence for clone | 63690088 R0663: E02 |
| SEQ ID NO: 274 | is the determined cDNA sequence for clone | 63690089 R0663: E03 |
| SEQ ID NO: 275 | is the determined cDNA sequence for clone | 63690090 R0663: E04 |
| SEQ ID NO: 276 | is the determined cDNA sequence for clone | 63690091 R0663: E05 |
| SEQ ID NO: 277 | is the determined cDNA sequence for clone | 63690092 R0663: E06 |
| SEQ ID NO: 278 | is the determined cDNA sequence for clone | 63690094 R0663: E08 |
| SEQ ID NO: 279 | is the determined cDNA sequence for clone | 63690095 R0663: E09 |
| SEQ ID NO: 280 | is the determined cDNA sequence for clone | 63690096 R0663: E10 |
| SEQ ID NO: 281 | is the determined cDNA sequence for clone | 63690097 R0663: E11 |
| SEQ ID NO: 282 | is the determined cDNA sequence for clone | 63690098 R0663: E12 |
| SEQ ID NO: 283 | is the determined cDNA sequence for clone | 63690099 R0663: F01 |
| SEQ ID NO: 284 | is the determined cDNA sequence for clone | 63690100 R0663: F02 |
| SEQ ID NO: 285 | is the determined cDNA sequence for clone | 63690101 R0663: F03 |
| SEQ ID NO: 286 | is the determined cDNA sequence for clone | 63690102 R0663: F04 |
| SEQ ID NO: 287 | is the determined cDNA sequence for clone | 63690104 R0663: F06 |
| SEQ ID NO: 288 | is the determined cDNA sequence for clone | 63690105 R0663: F07 |
| SEQ ID NO: 289 | is the determined cDNA sequence for clone | 63690106 R0663: F08 |
| SEQ ID NO: 290 | is the determined cDNA sequence for clone | 63690107 R0663: F09 |
| SEQ ID NO: 291 | is the determined cDNA sequence for clone | 63690108 R0663: F10 |
| SEQ ID NO: 292 | is the determined cDNA sequence for clone | 63690109 R0663: F11 |
| SEQ ID NO: 293 | is the determined cDNA sequence for clone | 63690110 R0663: F12 |
| SEQ ID NO: 294 | is the determined cDNA sequence for clone | 63690111 R0663: G01 |
| SEQ ID NO: 295 | is the determined cDNA sequence for clone | 63690112 R0663: G02 |
| SEQ ID NO: 296 | is the determined cDNA sequence for clone | 63690114 R0663: G04 |
| SEQ ID NO: 297 | is the determined cDNA sequence for clone | 63690115 R0663: G05 |
| SEQ ID NO: 298 | is the determined cDNA sequence for clone | 63690116 R0663: G06 |
| SEQ ID NO: 299 | is the determined cDNA sequence for clone | 63690117 R0663: G07 |
| SEQ ID NO: 300 | is the determined cDNA sequence for clone | 63690118 R0663: G08 |
| SEQ ID NO: 301 | is the determined cDNA sequence for clone | 63690119 R0663: G09 |
| SEQ ID NO: 302 | is the determined cDNA sequence for clone | 63690121 R0663: G11 |
| SEQ ID NO: 303 | is the determined cDNA sequence for clone | 63690122 R0663: G12 |
| SEQ ID NO: 304 | is the determined cDNA sequence for clone | 63690123 R0663: H01 |
| SEQ ID NO: 305 | is the determined cDNA sequence for clone | 63690124 R0663: H02 |
| SEQ ID NO: 306 | is the determined cDNA sequence for clone | 63690125 R0663: H03 |
| SEQ ID NO: 307 | is the determined cDNA sequence for clone | 63690126 R0663: H04 |
| SEQ ID NO: 308 | is the determined cDNA sequence for clone | 63690127 R0663: H05 |
| SEQ ID NO: 309 | is the determined cDNA sequence for clone | 63690128 R0663: H06 |
| SEQ ID NO: 310 | is the determined cDNA sequence for clone | 63690129 R0663: H07 |
| SEQ ID NO: 311 | is the determined cDNA sequence for clone | 63690130 R0663: H08 |
| SEQ ID NO: 312 | is the determined cDNA sequence for clone | 63690131 R0663: H09 |
| SEQ ID NO: 313 | is the determined cDNA sequence for clone | 63690132 R0663: H10 |
| SEQ ID NO: 314 | is the determined cDNA sequence for clone | 63690133 R0663: H11 |
| SEQ ID NO: 315 | is the determined cDNA sequence for clone | 63689948 R0664: A02 |
| SEQ ID NO: 316 | is the determined cDNA sequence for clone | 63689949 R0664: A03 |
| SEQ ID NO: 317 | is the determined cDNA sequence for clone | 63689950 R0664: A05 |
| SEQ ID NO: 318 | is the determined cDNA sequence for clone | 63689951 R0664: A06 |
| SEQ ID NO: 319 | is the determined cDNA sequence for clone | 63689952 R0664: A07 |
| SEQ ID NO: 320 | is the determined cDNA sequence for clone | 63689953 R0664: A08 |
| SEQ ID NO: 321 | is the determined cDNA sequence for clone | 63689954 R0664: A09 |
| SEQ ID NO: 322 | is the determined cDNA sequence for clone | 63689956 R0664: A11 |
| SEQ ID NO: 323 | is the determined cDNA sequence for clone | 63689957 R0664: A12 |
| SEQ ID NO: 324 | is the determined cDNA sequence for clone | 63689959 R0664: B02 |
| SEQ ID NO: 325 | is the determined cDNA sequence for clone | 63689961 R0664: B04 |
| SEQ ID NO: 326 | is the determined cDNA sequence for clone | 63689962 R0664: B05 |
| SEQ ID NO: 327 | is the determined cDNA sequence for clone | 63689963 R0664: B06 |
| SEQ ID NO: 328 | is the determined cDNA sequence for clone | 63689964 R0664: B07 |
| SEQ ID NO: 329 | is the determined cDNA sequence for clone | 63689965 R0664: B08 |
| SEQ ID NO: 330 | is the determined cDNA sequence for clone | 63689966 R0664: B09 |
| SEQ ID NO: 331 | is the determined cDNA sequence for clone | 63689967 R0664: B10 |
| SEQ ID NO: 332 | is the determined cDNA sequence for clone | 63689968 R0664: B11 |
| SEQ ID NO: 333 | is the determined cDNA sequence for clone | 63689969 R0664: B12 |
| SEQ ID NO: 334 | is the determined cDNA sequence for clone | 63689970 R0664: C01 |
| SEQ ID NO: 335 | is the determined cDNA sequence for clone | 63689972 R0664: C03 |
| SEQ ID NO: 336 | is the determined cDNA sequence for clone | 63689973 R0664: C04 |
| SEQ ID NO: 337 | is the determined cDNA sequence for clone | 63689974 R0664: C05 |
| SEQ ID NO: 338 | is the determined cDNA sequence for clone | 63689975 R0664: C06 |
| SEQ ID NO: 339 | is the determined cDNA sequence for clone | 63689976 R0664: C07 |
| SEQ ID NO: 340 | is the determined cDNA sequence for clone | 63689977 R0664: C08 |
| SEQ ID NO: 341 | is the determined cDNA sequence for clone | 63689978 R0664: C09 |
| SEQ ID NO: 342 | is the determined cDNA sequence for clone | 63689979 R0664: C10 |
| SEQ ID NO: 343 | is the determined cDNA sequence for clone | 63689980 R0664: C11 |
| SEQ ID NO: 344 | is the determined cDNA sequence for clone | 63689981 R0664: C12 |
| SEQ ID NO: 345 | is the determined cDNA sequence for clone | 63689982 R0664: D01 |
| SEQ ID NO: 346 | is the determined cDNA sequence for clone | 63689983 R0664: D02 |
| SEQ ID NO: 347 | is the determined cDNA sequence for clone | 63689984 R0664: D03 |
| SEQ ID NO: 348 | is the determined cDNA sequence for clone | 63689985 R0664: D04 |
| SEQ ID NO: 349 | is the determined cDNA sequence for clone | 63689986 R0664: D05 |
| SEQ ID NO: 350 | is the determined cDNA sequence for clone | 63689987 R0664: D06 |
| SEQ ID NO: 351 | is the determined cDNA sequence for clone | 63689988 R0664: D07 |
| SEQ ID NO: 352 | is the determined cDNA sequence for clone | 63689990 R0664: D09 |
| SEQ ID NO: 353 | is the determined cDNA sequence for clone | 63689992 R0664: D11 |
| SEQ ID NO: 354 | is the determined cDNA sequence for clone | 63689993 R0664: D12 |
| SEQ ID NO: 355 | is the determined cDNA sequence for clone | 63689994 R0664: E01 |
| SEQ ID NO: 356 | is the determined cDNA sequence for clone | 63689995 R0664: E02 |
| SEQ ID NO: 357 | is the determined cDNA sequence for clone | 63689996 R0664: E03 |
| SEQ ID NO: 358 | is the determined cDNA sequence for clone | 63689997 R0664: E04 |
| SEQ ID NO: 359 | is the determined cDNA sequence for clone | 63689998 R0664: E05 |
| SEQ ID NO: 360 | is the determined cDNA sequence for clone | 63689999 R0664: E06 |
| SEQ ID NO: 361 | is the determined cDNA sequence for clone | 63690000 R0664: E07 |
| SEQ ID NO: 362 | is the determined cDNA sequence for clone | 63690001 R0664: E08 |
| SEQ ID NO: 363 | is the determined cDNA sequence for clone | 63690002 R0664: E09 |
| SEQ ID NO: 364 | is the determined cDNA sequence for clone | 63690003 R0664: E10 |
| SEQ ID NO: 365 | is the determined cDNA sequence for clone | 63690004 R0664: E11 |
| SEQ ID NO: 366 | is the determined cDNA sequence for clone | 63690006 R0664: F01 |
| SEQ ID NO: 367 | is the determined cDNA sequence for clone | 63690007 R0664: F02 |
| SEQ ID NO: 368 | is the determined cDNA sequence for clone | 63690008 R0664: F03 |
| SEQ ID NO: 369 | is the determined cDNA sequence for clone | 63690009 R0664: F04 |
| SEQ ID NO: 370 | is the determined cDNA sequence for clone | 63690010 R0664: F05 |
| SEQ ID NO: 371 | is the determined cDNA sequence for clone | 63690011 R0664: F06 |
| SEQ ID NO: 372 | is the determined cDNA sequence for clone | 63690012 R0664: F07 |
| SEQ ID NO: 373 | is the determined cDNA sequence for clone | 63690013 R0664: F08 |
| SEQ ID NO: 374 | is the determined cDNA sequence for clone | 63690014 R0664: F09 |
| SEQ ID NO: 375 | is the determined cDNA sequence for clone | 63690015 R0664: F10 |
| SEQ ID NO: 376 | is the determined cDNA sequence for clone | 63690016 R0664: F11 |
| SEQ ID NO: 377 | is the determined cDNA sequence for clone | 63690017 R0664: F12 |
| SEQ ID NO: 378 | is the determined cDNA sequence for clone | 63690030 R0664: H01 |
| SEQ ID NO: 379 | is the determined cDNA sequence for clone | 63690031 R0664: H02 |
| SEQ ID NO: 380 | is the determined cDNA sequence for clone | 63690032 R0664: H03 |
| SEQ ID NO: 381 | is the determined cDNA sequence for clone | 63690033 R0664: H04 |
| SEQ ID NO: 382 | is the determined cDNA sequence for clone | 63690034 R0664: H05 |
| SEQ ID NO: 383 | is the determined cDNA sequence for clone | 63690035 R0664: H06 |
| SEQ ID NO: 384 | is the determined cDNA sequence for clone | 63690037 R0664: H08 |
| SEQ ID NO: 385 | is the determined cDNA sequence for clone | 63690038 R0664: H09 |
| SEQ ID NO: 386 | is the determined cDNA sequence for clone | 63690040 R0664: H11 |
| SEQ ID NO: 387 | is the determined cDNA sequence for clone | 63689762 R0665: A02 |
| SEQ ID NO: 388 | is the determined cDNA sequence for clone | 63689763 R0665: A03 |
| SEQ ID NO: 389 | is the determined cDNA sequence for clone | 63689764 R0665: A05 |
| SEQ ID NO: 390 | is the determined cDNA sequence for clone | 63689765 R0665: A06 |
| SEQ ID NO: 391 | is the determined cDNA sequence for clone | 63689766 R0665: A07 |
| SEQ ID NO: 392 | is the determined cDNA sequence for clone | 63689767 R0665: A08 |
| SEQ ID NO: 393 | is the determined cDNA sequence for clone | 63689768 R0665: A09 |
| SEQ ID NO: 394 | is the determined cDNA sequence for clone | 63689769 R0665: A10 |
| SEQ ID NO: 395 | is the determined cDNA sequence for clone | 63689770 R0665: A11 |
| SEQ ID NO: 396 | is the determined cDNA sequence for clone | 63689771 R0665: A12 |
| SEQ ID NO: 397 | is the determined cDNA sequence for clone | 63689772 R0665: B01 |
| SEQ ID NO: 398 | is the determined cDNA sequence for clone | 63689773 R0665: B02 |
| SEQ ID NO: 399 | is the determined cDNA sequence for clone | 63689774 R0665: B03 |
| SEQ ID NO: 400 | is the determined cDNA sequence for clone | 63689775 R0665: B04 |
| SEQ ID NO: 401 | is the determined cDNA sequence for clone | 63689777 R0665: B06 |
| SEQ ID NO: 402 | is the determined cDNA sequence for clone | 63689778 R0665: B07 |
| SEQ ID NO: 403 | is the determined cDNA sequence for clone | 63689780 R0665: B09 |
| SEQ ID NO: 404 | is the determined cDNA sequence for clone | 63689781 R0665: B10 |
| SEQ ID NO: 405 | is the determined cDNA sequence for clone | 63689782 R0665: B11 |
| SEQ ID NO: 406 | is the determined cDNA sequence for clone | 63689783 R0665: B12 |
| SEQ ID NO: 407 | is the determined cDNA sequence for clone | 63689784 R0665: C01 |
| SEQ ID NO: 408 | is the determined cDNA sequence for clone | 63689785 R0665: C02 |
| SEQ ID NO: 409 | is the determined cDNA sequence for clone | 63689786 R0665: C03 |
| SEQ ID NO: 410 | is the determined cDNA sequence for clone | 63689788 R0665: C05 |
| SEQ ID NO: 411 | is the determined cDNA sequence for clone | 63689789 R0665: C06 |
| SEQ ID NO: 412 | is the determined cDNA sequence for clone | 63689790 R0665: C07 |
| SEQ ID NO: 413 | is the determined cDNA sequence for clone | 63689791 R0665: C08 |
| SEQ ID NO: 414 | is the determined cDNA sequence for clone | 63689792 R0665: C09 |
| SEQ ID NO: 415 | is the determined cDNA sequence for clone | 63689793 R0665: C10 |
| SEQ ID NO: 416 | is the determined cDNA sequence for clone | 63689794 R0665: C11 |
| SEQ ID NO: 417 | is the determined cDNA sequence for clone | 63689795 R0665: C12 |
| SEQ ID NO: 418 | is the determined cDNA sequence for clone | 63689797 R0665: D02 |
| SEQ ID NO: 419 | is the determined cDNA sequence for clone | 63689798 R0665: D03 |
| SEQ ID NO: 420 | is the determined cDNA sequence for clone | 63689799 R0665: D04 |
| SEQ ID NO: 421 | is the determined cDNA sequence for clone | 63689801 R0665: D06 |
| SEQ ID NO: 422 | is the determined cDNA sequence for clone | 63689802 R0665: D07 |
| SEQ ID NO: 423 | is the determined cDNA sequence for clone | 63689804 R0665: D09 |
| SEQ ID NO: 424 | is the determined cDNA sequence for clone | 63689805 R0665: D10 |
| SEQ ID NO: 425 | is the determined cDNA sequence for clone | 63689806 R0665: D11 |
| SEQ ID NO: 426 | is the determined cDNA sequence for clone | 63689807 R0665: D12 |
| SEQ ID NO: 427 | is the determined cDNA sequence for clone | 63689808 R0665: E01 |
| SEQ ID NO: 428 | is the determined cDNA sequence for clone | 63689809 R0665: E02 |
| SEQ ID NO: 429 | is the determined cDNA sequence for clone | 63689810 R0665: E03 |
| SEQ ID NO: 430 | is the determined cDNA sequence for clone | 63689811 R0665: E04 |
| SEQ ID NO: 431 | is the determined cDNA sequence for clone | 63689812 R0665: E05 |
| SEQ ID NO: 432 | is the determined cDNA sequence for clone | 63689813 R0665: E06 |
| SEQ ID NO: 433 | is the determined cDNA sequence for clone | 63689814 R0665: E07 |
| SEQ ID NO: 434 | is the determined cDNA sequence for clone | 63689815 R0665: E08 |
| SEQ ID NO: 435 | is the determined cDNA sequence for clone | 63689816 R0665: E09 |
| SEQ ID NO: 436 | is the determined cDNA sequence for clone | 63689817 R0665: E10 |
| SEQ ID NO: 437 | is the determined cDNA sequence for clone | 63689818 R0665: E11 |
| SEQ ID NO: 438 | is the determined cDNA sequence for clone | 63689819 R0665: E12 |
| SEQ ID NO: 439 | is the determined cDNA sequence for clone | 63689820 R0665: F01 |
| SEQ ID NO: 440 | is the determined cDNA sequence for clone | 63689821 R0665: F02 |
| SEQ ID NO: 441 | is the determined cDNA sequence for clone | 63689824 R0665: F05 |
| SEQ ID NO: 442 | is the determined cDNA sequence for clone | 63689825 R0665: F06 |
| SEQ ID NO: 443 | is the determined cDNA sequence for clone | 63689826 R0665: F07 |
| SEQ ID NO: 444 | is the determined cDNA sequence for clone | 63689827 R0665: F08 |
| SEQ ID NO: 445 | is the determined cDNA sequence for clone | 63689828 R0665: F09 |
| SEQ ID NO: 446 | is the determined cDNA sequence for clone | 63689829 R0665: F10 |
| SEQ ID NO: 447 | is the determined cDNA sequence for clone | 63689830 R0665: F11 |
| SEQ ID NO: 448 | is the determined cDNA sequence for clone | 63689832 R0665: G01 |
| SEQ ID NO: 449 | is the determined cDNA sequence for clone | 63689833 R0665: G02 |
| SEQ ID NO: 450 | is the determined cDNA sequence for clone | 63689834 R0665: G03 |
| SEQ ID NO: 451 | is the determined cDNA sequence for clone | 63689837 R0665: G06 |
| SEQ ID NO: 452 | is the determined cDNA sequence for clone | 63689838 R0665: G07 |
| SEQ ID NO: 453 | is the determined cDNA sequence for clone | 63689839 R0665: G08 |
| SEQ ID NO: 454 | is the determined cDNA sequence for clone | 63689840 R0665: G09 |
| SEQ ID NO: 455 | is the determined cDNA sequence for clone | 63689842 R0665: G11 |
| SEQ ID NO: 456 | is the determined cDNA sequence for clone | 63689843 R0665: G12 |
| SEQ ID NO: 457 | is the determined cDNA sequence for clone | 63689845 R0665: H02 |
| SEQ ID NO: 458 | is the determined cDNA sequence for clone | 63689846 R0665: H03 |
| SEQ ID NO: 459 | is the determined cDNA sequence for clone | 63689847 R0665: H04 |
| SEQ ID NO: 460 | is the determined cDNA sequence for clone | 63689848 R0665: H05 |
| SEQ ID NO: 461 | is the determined cDNA sequence for clone | 63689849 R0665: H06 |
| SEQ ID NO: 462 | is the determined cDNA sequence for clone | 63689850 R0665: H07 |
| SEQ ID NO: 463 | is the determined cDNA sequence for clone | 63689851 R0665: H08 |
| SEQ ID NO: 464 | is the determined cDNA sequence for clone | 63689852 R0665: H09 |
| SEQ ID NO: 465 | is the determined cDNA sequence for clone | 63689853 R0665: H10 |
| SEQ ID NO: 466 | is the determined cDNA sequence for clone | 63689854 R0665: H11 |
| SEQ ID NO: 467 | is the determined cDNA sequence for clone | 63689577 R0666: A03 |
| SEQ ID NO: 468 | is the determined cDNA sequence for clone | 63689578 R0666: A05 |
| SEQ ID NO: 469 | is the determined cDNA sequence for clone | 63689579 R0666: A06 |
| SEQ ID NO: 470 | is the determined cDNA sequence for clone | 63689580 R0666: A07 |
| SEQ ID NO: 471 | is the determined cDNA sequence for clone | 63689581 R0666: A08 |
| SEQ ID NO: 472 | is the determined cDNA sequence for clone | 63689582 R0666: A09 |
| SEQ ID NO: 473 | is the determined cDNA sequence for clone | 63689583 R0666: A10 |
| SEQ ID NO: 474 | is the determined cDNA sequence for clone | 63689584 R0666: A11 |
| SEQ ID NO: 475 | is the determined cDNA sequence for clone | 63689585 R0666: A12 |
| SEQ ID NO: 476 | is the determined cDNA sequence for clone | 63689586 R0666: B01 |
| SEQ ID NO: 477 | is the determined cDNA sequence for clone | 63689587 R0666: B02 |
| SEQ ID NO: 478 | is the determined cDNA sequence for clone | 63689590 R0666: B05 |
| SEQ ID NO: 479 | is the determined cDNA sequence for clone | 63689591 R0666: B06 |
| SEQ ID NO: 480 | is the determined cDNA sequence for clone | 63689592 R0666: B07 |
| SEQ ID NO: 481 | is the determined cDNA sequence for clone | 63689593 R0666: B08 |
| SEQ ID NO: 482 | is the determined cDNA sequence for clone | 63689594 R0666: B09 |
| SEQ ID NO: 483 | is the determined cDNA sequence for clone | 63689595 R0666: B10 |
| SEQ ID NO: 484 | is the determined cDNA sequence for clone | 63689596 R0666: B11 |
| SEQ ID NO: 485 | is the determined cDNA sequence for clone | 63689598 R0666: C01 |
| SEQ ID NO: 486 | is the determined cDNA sequence for clone | 63689600 R0666: C03 |
| SEQ ID NO: 487 | is the determined cDNA sequence for clone | 63689601 R0666: C04 |
| SEQ ID NO: 488 | is the determined cDNA sequence for clone | 63689602 R0666: C05 |
| SEQ ID NO: 489 | is the determined cDNA sequence for clone | 63689603 R0666: C06 |
| SEQ ID NO: 490 | is the determined cDNA sequence for clone | 63689606 R0666: C09 |
| SEQ ID NO: 491 | is the determined cDNA sequence for clone | 63689607 R0666: C10 |
| SEQ ID NO: 492 | is the determined cDNA sequence for clone | 63689608 R0666: C11 |
| SEQ ID NO: 493 | is the determined cDNA sequence for clone | 63689609 R0666: C12 |
| SEQ ID NO: 494 | is the determined cDNA sequence for clone | 63689610 R0666: D01 |
| SEQ ID NO: 495 | is the determined cDNA sequence for clone | 63689611 R0666: D02 |
| SEQ ID NO: 496 | is the determined cDNA sequence for clone | 63689612 R0666: D03 |
| SEQ ID NO: 497 | is the determined cDNA sequence for clone | 63689613 R0666: D04 |
| SEQ ID NO: 498 | is the determined cDNA sequence for clone | 63689614 R0666: D05 |
| SEQ ID NO: 499 | is the determined cDNA sequence for clone | 63689615 R0666: D06 |
| SEQ ID NO: 500 | is the determined cDNA sequence for clone | 63689616 R0666: D07 |
| SEQ ID NO: 501 | is the determined cDNA sequence for clone | 63689617 R0666: D08 |
| SEQ ID NO: 502 | is the determined cDNA sequence for clone | 63689618 R0666: D09 |
| SEQ ID NO: 503 | is the determined cDNA sequence for clone | 63689619 R0666: D10 |
| SEQ ID NO: 504 | is the determined cDNA sequence for clone | 63689620 R0666: D11 |
| SEQ ID NO: 505 | is the determined cDNA sequence for clone | 63689622 R0666: E01 |
| SEQ ID NO: 506 | is the determined cDNA sequence for clone | 63689624 R0666: E03 |
| SEQ ID NO: 507 | is the determined cDNA sequence for clone | 63689625 R0666: E04 |
| SEQ ID NO: 508 | is the determined cDNA sequence for clone | 63689626 R0666: E05 |
| SEQ ID NO: 509 | is the determined cDNA sequence for clone | 63689627 R0666: E06 |
| SEQ ID NO: 510 | is the determined cDNA sequence for clone | 63689628 R0666: E07 |
| SEQ ID NO: 511 | is the determined cDNA sequence for clone | 63689630 R0666: E09 |
| SEQ ID NO: 512 | is the determined cDNA sequence for clone | 63689631 R0666: E10 |
| SEQ ID NO: 513 | is the determined cDNA sequence for clone | 63689632 R0666: E11 |
| SEQ ID NO: 514 | is the determined cDNA sequence for clone | 63689633 R0666: E12 |
| SEQ ID NO: 515 | is the determined cDNA sequence for clone | 63689634 R0666: F01 |
| SEQ ID NO: 516 | is the determined cDNA sequence for clone | 63689635 R0666: F02 |
| SEQ ID NO: 517 | is the determined cDNA sequence for clone | 63689636 R0666: F03 |
| SEQ ID NO: 518 | is the determined cDNA sequence for clone | 63689637 R0666: F04 |
| SEQ ID NO: 519 | is the determined cDNA sequence for clone | 63689638 R0666: F05 |
| SEQ ID NO: 520 | is the determined cDNA sequence for clone | 63689639 R0666: F06 |
| SEQ ID NO: 521 | is the determined cDNA sequence for clone | 63689641 R0666: F08 |
| SEQ ID NO: 522 | is the determined cDNA sequence for clone | 63689642 R0666: F09 |
| SEQ ID NO: 523 | is the determined cDNA sequence for clone | 63689643 R0666: F10 |
| SEQ ID NO: 524 | is the determined cDNA sequence for clone | 63689644 R0666: F11 |
| SEQ ID NO: 525 | is the determined cDNA sequence for clone | 63689645 R0666: F12 |
| SEQ ID NO: 526 | is the determined cDNA sequence for clone | 63689648 R0666: G03 |
| SEQ ID NO: 527 | is the determined cDNA sequence for clone | 63689649 R0666: G04 |
| SEQ ID NO: 528 | is the determined cDNA sequence for clone | 63689650 R0666: G05 |
| SEQ ID NO: 529 | is the determined cDNA sequence for clone | 63689652 R0666: G07 |
| SEQ ID NO: 530 | is the determined cDNA sequence for clone | 63689653 R0666: G08 |
| SEQ ID NO: 531 | is the determined cDNA sequence for clone | 63689654 R0666: G09 |
| SEQ ID NO: 532 | is the determined cDNA sequence for clone | 63689655 R0666: G10 |
| SEQ ID NO: 533 | is the determined cDNA sequence for clone | 63689656 R0666: G11 |
| SEQ ID NO: 534 | is the determined cDNA sequence for clone | 63689658 R0666: H01 |
| SEQ ID NO: 535 | is the determined cDNA sequence for clone | 63689659 R0666: H02 |
| SEQ ID NO: 536 | is the determined cDNA sequence for clone | 63689660 R0666: H03 |
| SEQ ID NO: 537 | is the determined cDNA sequence for clone | 63689661 R0666: H04 |
| SEQ ID NO: 538 | is the determined cDNA sequence for clone | 63689662 R0666: H05 |
| SEQ ID NO: 539 | is the determined cDNA sequence for clone | 63689663 R0666: H06 |
| SEQ ID NO: 540 | is the determined cDNA sequence for clone | 63689664 R0666: H07 |
| SEQ ID NO: 541 | is the determined cDNA sequence for clone | 63689665 R0666: H08 |
| SEQ ID NO: 542 | is the determined cDNA sequence for clone | 63689666 R0666: H09 |
| SEQ ID NO: 543 | is the determined cDNA sequence for clone | 63689667 R0666: H10 |
| SEQ ID NO: 544 | is the determined cDNA sequence for clone | 63689668 R0666: H11 |
| SEQ ID NO: 545 | is the determined cDNA sequence for clone | 63689484 R0667: A03 |
| SEQ ID NO: 546 | is the determined cDNA sequence for clone | 63689485 R0667: A05 |
| SEQ ID NO: 547 | is the determined cDNA sequence for clone | 63689486 R0667: A06 |
| SEQ ID NO: 548 | is the determined cDNA sequence for clone | 63689487 R0667: A07 |
| SEQ ID NO: 549 | is the determined cDNA sequence for clone | 63689488 R0667: A08 |
| SEQ ID NO: 550 | is the determined cDNA sequence for clone | 63689489 R0667: A09 |
| SEQ ID NO: 551 | is the determined cDNA sequence for clone | 63689491 R0667: A11 |
| SEQ ID NO: 552 | is the determined cDNA sequence for clone | 63689492 R0667: A12 |
| SEQ ID NO: 553 | is the determined cDNA sequence for clone | 63689493 R0667: B01 |
| SEQ ID NO: 554 | is the determined cDNA sequence for clone | 63689494 R0667: B02 |
| SEQ ID NO: 555 | is the determined cDNA sequence for clone | 63689495 R0667: B03 |
| SEQ ID NO: 556 | is the determined cDNA sequence for clone | 63689496 R0667: B04 |
| SEQ ID NO: 557 | is the determined cDNA sequence for clone | 63689497 R0667: B05 |
| SEQ ID NO: 558 | is the determined cDNA sequence for clone | 63689498 R0667: B06 |
| SEQ ID NO: 559 | is the determined cDNA sequence for clone | 63689499 R0667: B07 |
| SEQ ID NO: 560 | is the determined cDNA sequence for clone | 63689500 R0667: B08 |
| SEQ ID NO: 561 | is the determined cDNA sequence for clone | 63689501 R0667: B09 |
| SEQ ID NO: 562 | is the determined cDNA sequence for clone | 63689502 R0667: B10 |
| SEQ ID NO: 563 | is the determined cDNA sequence for clone | 63689503 R0667: B11 |
| SEQ ID NO: 564 | is the determined cDNA sequence for clone | 63689504 R0667: B12 |
| SEQ ID NO: 565 | is the determined cDNA sequence for clone | 63689505 R0667: C01 |
| SEQ ID NO: 566 | is the determined cDNA sequence for clone | 63689506 R0667: C02 |
| SEQ ID NO: 567 | is the determined cDNA sequence for clone | 63689507 R0667: C03 |
| SEQ ID NO: 568 | is the determined cDNA sequence for clone | 63689508 R0667: C04 |
| SEQ ID NO: 569 | is the determined cDNA sequence for clone | 63689509 R0667: C05 |
| SEQ ID NO: 570 | is the determined cDNA sequence for clone | 63689511 R0667: C07 |
| SEQ ID NO: 571 | is the determined cDNA sequence for clone | 63689512 R0667: C08 |
| SEQ ID NO: 572 | is the determined cDNA sequence for clone | 63689514 R0667: C10 |
| SEQ ID NO: 573 | is the determined cDNA sequence for clone | 63689515 R0667: C11 |
| SEQ ID NO: 574 | is the determined cDNA sequence for clone | 63689516 R0667: C12 |
| SEQ ID NO: 575 | is the determined cDNA sequence for clone | 63689517 R0667: D01 |
| SEQ ID NO: 576 | is the determined cDNA sequence for clone | 63689518 R0667: D02 |
| SEQ ID NO: 577 | is the determined cDNA sequence for clone | 63689519 R0667: D03 |
| SEQ ID NO: 578 | is the determined cDNA sequence for clone | 63689520 R0667: D04 |
| SEQ ID NO: 579 | is the determined cDNA sequence for clone | 63689521 R0667: D05 |
| SEQ ID NO: 580 | is the determined cDNA sequence for clone | 63689522 R0667: D06 |
| SEQ ID NO: 581 | is the determined cDNA sequence for clone | 63689523 R0667: D07 |
| SEQ ID NO: 582 | is the determined cDNA sequence for clone | 63689524 R0667: D08 |
| SEQ ID NO: 583 | is the determined cDNA sequence for clone | 63689526 R0667: D10 |
| SEQ ID NO: 584 | is the determined cDNA sequence for clone | 63689527 R0667: D11 |
| SEQ ID NO: 585 | is the determined cDNA sequence for clone | 63689528 R0667: D12 |
| SEQ ID NO: 586 | is the determined cDNA sequence for clone | 63689529 R0667: E01 |
| SEQ ID NO: 587 | is the determined cDNA sequence for clone | 63689532 R0667: E04 |
| SEQ ID NO: 588 | is the determined cDNA sequence for clone | 63689533 R0667: E05 |
| SEQ ID NO: 589 | is the determined cDNA sequence for clone | 63689534 R0667: E06 |
| SEQ ID NO: 590 | is the determined cDNA sequence for clone | 63689535 R0667: E07 |
| SEQ ID NO: 591 | is the determined cDNA sequence for clone | 63689536 R0667: E08 |
| SEQ ID NO: 592 | is the determined cDNA sequence for clone | 63689537 R0667: E09 |
| SEQ ID NO: 593 | is the determined cDNA sequence for clone | 63689538 R0667: E10 |
| SEQ ID NO: 594 | is the determined cDNA sequence for clone | 63689539 R0667: E11 |
| SEQ ID NO: 595 | is the determined cDNA sequence for clone | 63689540 R0667: E12 |
| SEQ ID NO: 596 | is the determined cDNA sequence for clone | 63689541 R0667: F01 |
| SEQ ID NO: 597 | is the determined cDNA sequence for clone | 63689542 R0667: F02 |
| SEQ ID NO: 598 | is the determined cDNA sequence for clone | 63689544 R0667: F04 |
| SEQ ID NO: 599 | is the determined cDNA sequence for clone | 63689546 R0667: F06 |
| SEQ ID NO: 600 | is the determined cDNA sequence for clone | 63689547 R0667: F07 |
| SEQ ID NO: 601 | is the determined cDNA sequence for clone | 63689548 R0667: F08 |
| SEQ ID NO: 602 | is the determined cDNA sequence for clone | 63689549 R0667: F09 |
| SEQ ID NO: 603 | is the determined cDNA sequence for clone | 63689550 R0667: F10 |
| SEQ ID NO: 604 | is the determined cDNA sequence for clone | 63689551 R0667: F11 |
| SEQ ID NO: 605 | is the determined cDNA sequence for clone | 63689552 R0667: F12 |
| SEQ ID NO: 606 | is the determined cDNA sequence for clone | 63689553 R0667: G01 |
| SEQ ID NO: 607 | is the determined cDNA sequence for clone | 63689554 R0667: G02 |
| SEQ ID NO: 608 | is the determined cDNA sequence for clone | 63689555 R0667: G03 |
| SEQ ID NO: 609 | is the determined cDNA sequence for clone | 63689557 R0667: G05 |
| SEQ ID NO: 610 | is the determined cDNA sequence for clone | 63689558 R0667: G06 |
| SEQ ID NO: 611 | is the determined cDNA sequence for clone | 63689559 R0667: G07 |
| SEQ ID NO: 612 | is the determined cDNA sequence for clone | 63689560 R0667: G08 |
| SEQ ID NO: 613 | is the determined cDNA sequence for clone | 63689561 R0667: G09 |
| SEQ ID NO: 614 | is the determined cDNA sequence for clone | 63689562 R0667: G10 |
| SEQ ID NO: 615 | is the determined cDNA sequence for clone | 63689563 R0667: G11 |
| SEQ ID NO: 616 | is the determined cDNA sequence for clone | 63689564 R0667: G12 |
| SEQ ID NO: 617 | is the determined cDNA sequence for clone | 63689565 R0667: H01 |
| SEQ ID NO: 618 | is the determined cDNA sequence for clone | 63689566 R0667: H02 |
| SEQ ID NO: 619 | is the determined cDNA sequence for clone | 63689569 R0667: H05 |
| SEQ ID NO: 620 | is the determined cDNA sequence for clone | 63689570 R0667: H06 |
| SEQ ID NO: 621 | is the determined cDNA sequence for clone | 63689571 R0667: H07 |
| SEQ ID NO: 622 | is the determined cDNA sequence for clone | 63689572 R0667: H08 |
| SEQ ID NO: 623 | is the determined cDNA sequence for clone | 63689573 R0667: H09 |
| SEQ ID NO: 624 | is the determined cDNA sequence for clone | 63689574 R0667: H10 |
| SEQ ID NO: 625 | is the determined cDNA sequence for clone | 63689575 R0667: H11 |
| SEQ ID NO: 626 | is the determined cDNA sequence for clone | 63689390 R0668: A02 |
| SEQ ID NO: 627 | is the determined cDNA sequence for clone | 63689391 R0668: A03 |
| SEQ ID NO: 628 | is the determined cDNA sequence for clone | 63689392 R0668: A05 |
| SEQ ID NO: 629 | is the determined cDNA sequence for clone | 63689393 R0668: A06 |
| SEQ ID NO: 630 | is the determined cDNA sequence for clone | 63689394 R0668: A07 |
| SEQ ID NO: 631 | is the determined cDNA sequence for clone | 63689395 R0668: A08 |
| SEQ ID NO: 632 | is the determined cDNA sequence for clone | 63689396 R0668: A09 |
| SEQ ID NO: 633 | is the determined cDNA sequence for clone | 63689397 R0668: A10 |
| SEQ ID NO: 634 | is the determined cDNA sequence for clone | 63689398 R0668: A11 |
| SEQ ID NO: 635 | is the determined cDNA sequence for clone | 63689399 R0668: A12 |
| SEQ ID NO: 636 | is the determined cDNA sequence for clone | 63689401 R0668: B02 |
| SEQ ID NO: 637 | is the determined cDNA sequence for clone | 63689402 R0668: B03 |
| SEQ ID NO: 638 | is the determined cDNA sequence for clone | 63689403 R0668: B04 |
| SEQ ID NO: 639 | is the determined cDNA sequence for clone | 63689404 R0668: B05 |
| SEQ ID NO: 640 | is the determined cDNA sequence for clone | 63689405 R0668: B06 |
| SEQ ID NO: 641 | is the determined cDNA sequence for clone | 63689406 R0668: B07 |
| SEQ ID NO: 642 | is the determined cDNA sequence for clone | 63689407 R0668: B08 |
| SEQ ID NO: 643 | is the determined cDNA sequence for clone | 63689408 R0668: B09 |
| SEQ ID NO: 644 | is the determined cDNA sequence for clone | 63689409 R0668: B10 |
| SEQ ID NO: 645 | is the determined cDNA sequence for clone | 63689410 R0668: B11 |
| SEQ ID NO: 646 | is the determined cDNA sequence for clone | 63689411 R0668: B12 |
| SEQ ID NO: 647 | is the determined cDNA sequence for clone | 63689412 R0668: C01 |
| SEQ ID NO: 648 | is the determined cDNA sequence for clone | 63689413 R0668: C02 |
| SEQ ID NO: 649 | is the determined cDNA sequence for clone | 63689414 R0668: C03 |
| SEQ ID NO: 650 | is the determined cDNA sequence for clone | 63689415 R0668: C04 |
| SEQ ID NO: 651 | is the determined cDNA sequence for clone | 63689416 R0668: C05 |
| SEQ ID NO: 652 | is the determined cDNA sequence for clone | 63689417 R0668: C06 |
| SEQ ID NO: 653 | is the determined cDNA sequence for clone | 63689418 R0668: C07 |
| SEQ ID NO: 654 | is the determined cDNA sequence for clone | 63689419 R0668: C08 |
| SEQ ID NO: 655 | is the determined cDNA sequence for clone | 63689420 R0668: C09 |
| SEQ ID NO: 656 | is the determined cDNA sequence for clone | 63689421 R0668: C10 |
| SEQ ID NO: 657 | is the determined cDNA sequence for clone | 63689422 R0668: C11 |
| SEQ ID NO: 658 | is the determined cDNA sequence for clone | 63689423 R0668: C12 |
| SEQ ID NO: 659 | is the determined cDNA sequence for clone | 63689424 R0668: D01 |
| SEQ ID NO: 660 | is the determined cDNA sequence for clone | 63689425 R0668: D02 |
| SEQ ID NO: 661 | is the determined cDNA sequence for clone | 63689426 R0668: D03 |
| SEQ ID NO: 662 | is the determined cDNA sequence for clone | 63689427 R0668: D04 |
| SEQ ID NO: 663 | is the determined cDNA sequence for clone | 63689428 R0668: D05 |
| SEQ ID NO: 664 | is the determined cDNA sequence for clone | 63689429 R0668: D06 |
| SEQ ID NO: 665 | is the determined cDNA sequence for clone | 63689430 R0668: D07 |
| SEQ ID NO: 666 | is the determined cDNA sequence for clone | 63689431 R0668: D08 |
| SEQ ID NO: 667 | is the determined cDNA sequence for clone | 63689432 R0668: D09 |
| SEQ ID NO: 668 | is the determined cDNA sequence for clone | 63689433 R0668: D10 |
| SEQ ID NO: 669 | is the determined cDNA sequence for clone | 63689434 R0668: D11 |
| SEQ ID NO: 670 | is the determined cDNA sequence for clone | 63689435 R0668: D12 |
| SEQ ID NO: 671 | is the determined cDNA sequence for clone | 63689436 R0668: E01 |
| SEQ ID NO: 672 | is the determined cDNA sequence for clone | 63689437 R0668: E02 |
| SEQ ID NO: 673 | is the determined cDNA sequence for clone | 63689438 R0668: E03 |
| SEQ ID NO: 674 | is the determined cDNA sequence for clone | 63689439 R0668: E04 |
| SEQ ID NO: 675 | is the determined cDNA sequence for clone | 63689440 R0668: E05 |
| SEQ ID NO: 676 | is the determined cDNA sequence for clone | 63689441 R0668: E06 |
| SEQ ID NO: 677 | is the determined cDNA sequence for clone | 63689442 R0668: E07 |
| SEQ ID NO: 678 | is the determined cDNA sequence for clone | 63689443 R0668: E08 |
| SEQ ID NO: 679 | is the determined cDNA sequence for clone | 63689444 R0668: E09 |
| SEQ ID NO: 680 | is the determined cDNA sequence for clone | 63689446 R0668: E11 |
| SEQ ID NO: 681 | is the determined cDNA sequence for clone | 63689447 R0668: E12 |
| SEQ ID NO: 682 | is the determined cDNA sequence for clone | 63689450 R0668: F03 |
| SEQ ID NO: 683 | is the determined cDNA sequence for clone | 63689451 R0668: F04 |
| SEQ ID NO: 684 | is the determined cDNA sequence for clone | 63689452 R0668: F05 |
| SEQ ID NO: 685 | is the determined cDNA sequence for clone | 63689453 R0668: F06 |
| SEQ ID NO: 686 | is the determined cDNA sequence for clone | 63689454 R0668: F07 |
| SEQ ID NO: 687 | is the determined cDNA sequence for clone | 63689455 R0668: F08 |
| SEQ ID NO: 688 | is the determined cDNA sequence for clone | 63689456 R0668: F09 |
| SEQ ID NO: 689 | is the determined cDNA sequence for clone | 63689457 R0668: F10 |
| SEQ ID NO: 690 | is the determined cDNA sequence for clone | 63689458 R0668: F11 |
| SEQ ID NO: 691 | is the determined cDNA sequence for clone | 63689459 R0668: F12 |
| SEQ ID NO: 692 | is the determined cDNA sequence for clone | 63689460 R0668: G01 |
| SEQ ID NO: 693 | is the determined cDNA sequence for clone | 63689461 R0668: G02 |
| SEQ ID NO: 694 | is the determined cDNA sequence for clone | 63689462 R0668: G03 |
| SEQ ID NO: 695 | is the determined cDNA sequence for clone | 63689463 R0668: G04 |
| SEQ ID NO: 696 | is the determined cDNA sequence for clone | 63689464 R0668: G05 |
| SEQ ID NO: 697 | is the determined cDNA sequence for clone | 63689465 R0668: G06 |
| SEQ ID NO: 698 | is the determined cDNA sequence for clone | 63689466 R0668: G07 |
| SEQ ID NO: 699 | is the determined cDNA sequence for clone | 63689467 R0668: G08 |
| SEQ ID NO: 700 | is the determined cDNA sequence for clone | 63689468 R0668: G09 |
| SEQ ID NO: 701 | is the determined cDNA sequence for clone | 63689469 R0668: G10 |
| SEQ ID NO: 702 | is the determined cDNA sequence for clone | 63689470 R0668: G11 |
| SEQ ID NO: 703 | is the determined cDNA sequence for clone | 63689471 R0668: G12 |
| SEQ ID NO: 704 | is the determined cDNA sequence for clone | 63689474 R0668: H03 |
| SEQ ID NO: 705 | is the determined cDNA sequence for clone | 63689476 R0668: H05 |
| SEQ ID NO: 706 | is the determined cDNA sequence for clone | 63689477 R0668: H06 |
| SEQ ID NO: 707 | is the determined cDNA sequence for clone | 63689478 R0668: H07 |
| SEQ ID NO: 708 | is the determined cDNA sequence for clone | 63689479 R0668: H08 |
| SEQ ID NO: 709 | is the determined cDNA sequence for clone | 63689480 R0668: H09 |
| SEQ ID NO: 710 | is the determined cDNA sequence for clone | 63689481 R0668: H10 |
| SEQ ID NO: 711 | is the determined cDNA sequence for clone | 63689482 R0668: H11 |
| SEQ ID NO: 712 | is the determined cDNA sequence for clone | 63690135 R0669: A03 |
| SEQ ID NO: 713 | is the determined cDNA sequence for clone | 63690137 R0669: A06 |
| SEQ ID NO: 714 | is the determined cDNA sequence for clone | 63690139 R0669: A08 |
| SEQ ID NO: 715 | is the determined cDNA sequence for clone | 63690140 R0669: A09 |
| SEQ ID NO: 716 | is the determined cDNA sequence for clone | 63690141 R0669: A10 |
| SEQ ID NO: 717 | is the determined cDNA sequence for clone | 63690142 R0669: A11 |
| SEQ ID NO: 718 | is the determined cDNA sequence for clone | 63690143 R0669: A12 |
| SEQ ID NO: 719 | is the determined cDNA sequence for clone | 63690146 R0669: B03 |
| SEQ ID NO: 720 | is the determined cDNA sequence for clone | 63690147 R0669: B04 |
| SEQ ID NO: 721 | is the determined cDNA sequence for clone | 63690148 R0669: B05 |
| SEQ ID NO: 722 | is the determined cDNA sequence for clone | 63690149 R0669: B06 |
| SEQ ID NO: 723 | is the determined cDNA sequence for clone | 63690150 R0669: B07 |
| SEQ ID NO: 724 | is the determined cDNA sequence for clone | 63690151 R0669: B08 |
| SEQ ID NO: 725 | is the determined cDNA sequence for clone | 63690152 R0669: B09 |
| SEQ ID NO: 726 | is the determined cDNA sequence for clone | 63690153 R0669: B10 |
| SEQ ID NO: 727 | is the determined cDNA sequence for clone | 63690154 R0669: B11 |
| SEQ ID NO: 728 | is the determined cDNA sequence for clone | 63690155 R0669: B12 |
| SEQ ID NO: 729 | is the determined cDNA sequence for clone | 63690156 R0669: C01 |
| SEQ ID NO: 730 | is the determined cDNA sequence for clone | 63690157 R0669: C02 |
| SEQ ID NO: 731 | is the determined cDNA sequence for clone | 63690158 R0669: C03 |
| SEQ ID NO: 732 | is the determined cDNA sequence for clone | 63690159 R0669: C04 |
| SEQ ID NO: 733 | is the determined cDNA sequence for clone | 63690160 R0669: C05 |
| SEQ ID NO: 734 | is the determined cDNA sequence for clone | 63690161 R0669: C06 |
| SEQ ID NO: 735 | is the determined cDNA sequence for clone | 63690162 R0669: C07 |
| SEQ ID NO: 736 | is the determined cDNA sequence for clone | 63690163 R0669: C08 |
| SEQ ID NO: 737 | is the determined cDNA sequence for clone | 63690164 R0669: C09 |
| SEQ ID NO: 738 | is the determined cDNA sequence for clone | 63690165 R0669: C10 |
| SEQ ID NO: 739 | is the determined cDNA sequence for clone | 63690166 R0669: C11 |
| SEQ ID NO: 740 | is the determined cDNA sequence for clone | 63690167 R0669: C12 |
| SEQ ID NO: 741 | is the determined cDNA sequence for clone | 63690168 R0669: D01 |
| SEQ ID NO: 742 | is the determined cDNA sequence for clone | 63690169 R0669: D02 |
| SEQ ID NO: 743 | is the determined cDNA sequence for clone | 63690170 R0669: D03 |
| SEQ ID NO: 744 | is the determined cDNA sequence for clone | 63690171 R0669: D04 |
| SEQ ID NO: 745 | is the determined cDNA sequence for clone | 63690172 R0669: D05 |
| SEQ ID NO: 746 | is the determined cDNA sequence for clone | 63690173 R0669: D06 |
| SEQ ID NO: 747 | is the determined cDNA sequence for clone | 63690174 R0669: D07 |
| SEQ ID NO: 748 | is the determined cDNA sequence for clone | 63690175 R0669: D08 |
| SEQ ID NO: 749 | is the determined cDNA sequence for clone | 63690176 R0669: D09 |
| SEQ ID NO: 750 | is the determined cDNA sequence for clone | 63690177 R0669: D10 |
| SEQ ID NO: 751 | is the determined cDNA sequence for clone | 63690178 R0669: D11 |
| SEQ ID NO: 752 | is the determined cDNA sequence for clone | 63690179 R0669: D12 |
| SEQ ID NO: 753 | is the determined cDNA sequence for clone | 63690180 R0669: E01 |
| SEQ ID NO: 754 | is the determined cDNA sequence for clone | 63690181 R0669: E02 |
| SEQ ID NO: 755 | is the determined cDNA sequence for clone | 63690182 R0669: E03 |
| SEQ ID NO: 756 | is the determined cDNA sequence for clone | 63690183 R0669: E04 |
| SEQ ID NO: 757 | is the determined cDNA sequence for clone | 63690184 R0669: E05 |
| SEQ ID NO: 758 | is the determined cDNA sequence for clone | 63690185 R0669: E06 |
| SEQ ID NO: 759 | is the determined cDNA sequence for clone | 63690186 R0669: E07 |
| SEQ ID NO: 760 | is the determined cDNA sequence for clone | 63690187 R0669: E08 |
| SEQ ID NO: 761 | is the determined cDNA sequence for clone | 63690188 R0669: E09 |
| SEQ ID NO: 762 | is the determined cDNA sequence for clone | 63690189 R0669: E10 |
| SEQ ID NO: 763 | is the determined cDNA sequence for clone | 63690190 R0669: E11 |
| SEQ ID NO: 764 | is the determined cDNA sequence for clone | 63690191 R0669: E12 |
| SEQ ID NO: 765 | is the determined cDNA sequence for clone | 63690192 R0669: F01 |
| SEQ ID NO: 766 | is the determined cDNA sequence for clone | 63690193 R0669: F02 |
| SEQ ID NO: 767 | is the determined cDNA sequence for clone | 63690194 R0669: F03 |
| SEQ ID NO: 768 | is the determined cDNA sequence for clone | 63690195 R0669: F04 |
| SEQ ID NO: 769 | is the determined cDNA sequence for clone | 63690196 R0669: F05 |
| SEQ ID NO: 770 | is the determined cDNA sequence for clone | 63690197 R0669: F06 |
| SEQ ID NO: 771 | is the determined cDNA sequence for clone | 63690198 R0669: F07 |
| SEQ ID NO: 772 | is the determined cDNA sequence for clone | 63690199 R0669: F08 |
| SEQ ID NO: 773 | is the determined cDNA sequence for clone | 63690200 R0669: F09 |
| SEQ ID NO: 774 | is the determined cDNA sequence for clone | 63690201 R0669: F10 |
| SEQ ID NO: 775 | is the determined cDNA sequence for clone | 63690202 R0669: F11 |
| SEQ ID NO: 776 | is the determined cDNA sequence for clone | 63690203 R0669: F12 |
| SEQ ID NO: 777 | is the determined cDNA sequence for clone | 63690204 R0669: G01 |
| SEQ ID NO: 778 | is the determined cDNA sequence for clone | 63690205 R0669: G02 |
| SEQ ID NO: 779 | is the determined cDNA sequence for clone | 63690206 R0669: G03 |
| SEQ ID NO: 780 | is the determined cDNA sequence for clone | 63690208 R0669: G05 |
| SEQ ID NO: 781 | is the determined cDNA sequence for clone | 63690210 R0669: G07 |
| SEQ ID NO: 782 | is the determined cDNA sequence for clone | 63690211 R0669: G08 |
| SEQ ID NO: 783 | is the determined cDNA sequence for clone | 63690212 R0669: G09 |
| SEQ ID NO: 784 | is the determined cDNA sequence for clone | 63690213 R0669: G10 |
| SEQ ID NO: 785 | is the determined cDNA sequence for clone | 63690214 R0669: G11 |
| SEQ ID NO: 786 | is the determined cDNA sequence for clone | 63690215 R0669: G12 |
| SEQ ID NO: 787 | is the determined cDNA sequence for clone | 63690216 R0669: H01 |
| SEQ ID NO: 788 | is the determined cDNA sequence for clone | 63690217 R0669: H02 |
| SEQ ID NO: 789 | is the determined cDNA sequence for clone | 63690218 R0669: H03 |
| SEQ ID NO: 790 | is the determined cDNA sequence for clone | 63690219 R0669: H04 |
| SEQ ID NO: 791 | is the determined cDNA sequence for clone | 63690220 R0669: H05 |
| SEQ ID NO: 792 | is the determined cDNA sequence for clone | 63690222 R0669: H07 |
| SEQ ID NO: 793 | is the determined cDNA sequence for clone | 63690223 R0669: H08 |
| SEQ ID NO: 794 | is the determined cDNA sequence for clone | 63690224 R0669: H09 |
| SEQ ID NO: 795 | is the determined cDNA sequence for clone | 63690225 R0669: H10 |
| SEQ ID NO: 796 | is the determined cDNA sequence for clone | 63690226 R0669: H11 |
| SEQ ID NO: 797 | is the determined cDNA sequence for clone | 63695095 R0670: A02 |
| SEQ ID NO: 798 | is the determined cDNA sequence for clone | 63695097 R0670: A05 |
| SEQ ID NO: 799 | is the determined cDNA sequence for clone | 63695098 R0670: A06 |
| SEQ ID NO: 800 | is the determined cDNA sequence for clone | 63695099 R0670: A07 |
| SEQ ID NO: 801 | is the determined cDNA sequence for clone | 63695100 R0670: A08 |
| SEQ ID NO: 802 | is the determined cDNA sequence for clone | 63695101 R0670: A09 |
| SEQ ID NO: 803 | is the determined cDNA sequence for clone | 63695102 R0670: A10 |
| SEQ ID NO: 804 | is the determined cDNA sequence for clone | 63695103 R0670: A11 |
| SEQ ID NO: 805 | is the determined cDNA sequence for clone | 63695105 R0670: B01 |
| SEQ ID NO: 806 | is the determined cDNA sequence for clone | 63695107 R0670: B03 |
| SEQ ID NO: 807 | is the determined cDNA sequence for clone | 63695108 R0670: B04 |
| SEQ ID NO: 808 | is the determined cDNA sequence for clone | 63695109 R0670: B05 |
| SEQ ID NO: 809 | is the determined cDNA sequence for clone | 63695110 R0670: B06 |
| SEQ ID NO: 810 | is the determined cDNA sequence for clone | 63695111 R0670: B07 |
| SEQ ID NO: 811 | is the determined cDNA sequence for clone | 63695112 R0670: B08 |
| SEQ ID NO: 812 | is the determined cDNA sequence for clone | 63695113 R0670: B09 |
| SEQ ID NO: 813 | is the determined cDNA sequence for clone | 63695115 R0670: B11 |
| SEQ ID NO: 814 | is the determined cDNA sequence for clone | 63695116 R0670: B12 |
| SEQ ID NO: 815 | is the determined cDNA sequence for clone | 63695117 R0670: C01 |
| SEQ ID NO: 816 | is the determined cDNA sequence for clone | 63695118 R0670: C02 |
| SEQ ID NO: 817 | is the determined cDNA sequence for clone | 63695119 R0670: C03 |
| SEQ ID NO: 818 | is the determined cDNA sequence for clone | 63695120 R0670: C04 |
| SEQ ID NO: 819 | is the determined cDNA sequence for clone | 63695121 R0670: C05 |
| SEQ ID NO: 820 | is the determined cDNA sequence for clone | 63695122 R0670: C06 |
| SEQ ID NO: 821 | is the determined cDNA sequence for clone | 63695123 R0670: C07 |
| SEQ ID NO: 822 | is the determined cDNA sequence for clone | 63695124 R0670: C08 |
| SEQ ID NO: 823 | is the determined cDNA sequence for clone | 63695125 R0670: C09 |
| SEQ ID NO: 824 | is the determined cDNA sequence for clone | 63695126 R0670: C10 |
| SEQ ID NO: 825 | is the determined cDNA sequence for clone | 63695127 R0670: C11 |
| SEQ ID NO: 826 | is the determined cDNA sequence for clone | 63695128 R0670: C12 |
| SEQ ID NO: 827 | is the determined cDNA sequence for clone | 63695129 R0670: D01 |
| SEQ ID NO: 828 | is the determined cDNA sequence for clone | 63695130 R0670: D02 |
| SEQ ID NO: 829 | is the determined cDNA sequence for clone | 63695131 R0670: D03 |
| SEQ ID NO: 830 | is the determined cDNA sequence for clone | 63695132 R0670: D04 |
| SEQ ID NO: 831 | is the determined cDNA sequence for clone | 63695133 R0670: D05 |
| SEQ ID NO: 832 | is the determined cDNA sequence for clone | 63695134 R0670: D06 |
| SEQ ID NO: 833 | is the determined cDNA sequence for clone | 63695135 R0670: D07 |
| SEQ ID NO: 834 | is the determined cDNA sequence for clone | 63695136 R0670: D08 |
| SEQ ID NO: 835 | is the determined cDNA sequence for clone | 63695137 R0670: D09 |
| SEQ ID NO: 836 | is the determined cDNA sequence for clone | 63695138 R0670: D10 |
| SEQ ID NO: 837 | is the determined cDNA sequence for clone | 63695139 R0670: D11 |
| SEQ ID NO: 838 | is the determined cDNA sequence for clone | 63695140 R0670: D12 |
| SEQ ID NO: 839 | is the determined cDNA sequence for clone | 63695142 R0670: E02 |
| SEQ ID NO: 840 | is the determined cDNA sequence for clone | 63695143 R0670: E03 |
| SEQ ID NO: 841 | is the determined cDNA sequence for clone | 63695144 R0670: E04 |
| SEQ ID NO: 842 | is the determined cDNA sequence for clone | 63695145 R0670: E05 |
| SEQ ID NO: 843 | is the determined cDNA sequence for clone | 63695147 R0670: E07 |
| SEQ ID NO: 844 | is the determined cDNA sequence for clone | 63695148 R0670: E08 |
| SEQ ID NO: 845 | is the determined cDNA sequence for clone | 63695149 R0670: E09 |
| SEQ ID NO: 846 | is the determined cDNA sequence for clone | 63695150 R0670: E10 |
| SEQ ID NO: 847 | is the determined cDNA sequence for clone | 63695151 R0670: E11 |
| SEQ ID NO: 848 | is the determined cDNA sequence for clone | 63695152 R0670: E12 |
| SEQ ID NO: 849 | is the determined cDNA sequence for clone | 63695153 R0670: F01 |
| SEQ ID NO: 850 | is the determined cDNA sequence for clone | 63695154 R0670: F02 |
| SEQ ID NO: 851 | is the determined cDNA sequence for clone | 63695155 R0670: F03 |
| SEQ ID NO: 852 | is the determined cDNA sequence for clone | 63695156 R0670: F04 |
| SEQ ID NO: 853 | is the determined cDNA sequence for clone | 63695157 R0670: F05 |
| SEQ ID NO: 854 | is the determined cDNA sequence for clone | 63695158 R0670: F06 |
| SEQ ID NO: 855 | is the determined cDNA sequence for clone | 63695159 R0670: F07 |
| SEQ ID NO: 856 | is the determined cDNA sequence for clone | 63695160 R0670: F08 |
| SEQ ID NO: 857 | is the determined cDNA sequence for clone | 63695161 R0670: F09 |
| SEQ ID NO: 858 | is the determined cDNA sequence for clone | 63695162 R0670: F10 |
| SEQ ID NO: 859 | is the determined cDNA sequence for clone | 63695163 R0670: F11 |
| SEQ ID NO: 860 | is the determined cDNA sequence for clone | 63695164 R0670: F12 |
| SEQ ID NO: 861 | is the determined cDNA sequence for clone | 63695165 R0670: G01 |
| SEQ ID NO: 862 | is the determined cDNA sequence for clone | 63695166 R0670: G02 |
| SEQ ID NO: 863 | is the determined cDNA sequence for clone | 63695167 R0670: G03 |
| SEQ ID NO: 864 | is the determined cDNA sequence for clone | 63695168 R0670: G04 |
| SEQ ID NO: 865 | is the determined cDNA sequence for clone | 63695169 R0670: G05 |
| SEQ ID NO: 866 | is the determined cDNA sequence for clone | 63695170 R0670: G06 |
| SEQ ID NO: 867 | is the determined cDNA sequence for clone | 63695171 R0670: G07 |
| SEQ ID NO: 868 | is the determined cDNA sequence for clone | 63695172 R0670: G08 |
| SEQ ID NO: 869 | is the determined cDNA sequence for clone | 63695173 R0670: G09 |
| SEQ ID NO: 870 | is the determined cDNA sequence for clone | 63695174 R0670: G10 |
| SEQ ID NO: 871 | is the determined cDNA sequence for clone | 63695175 R0670: G11 |
| SEQ ID NO: 872 | is the determined cDNA sequence for clone | 63695176 R0670: G12 |
| SEQ ID NO: 873 | is the determined cDNA sequence for clone | 63695177 R0670: H01 |
| SEQ ID NO: 874 | is the determined cDNA sequence for clone | 63695178 R0670: H02 |
| SEQ ID NO: 875 | is the determined cDNA sequence for clone | 63695179 R0670: H03 |
| SEQ ID NO: 876 | is the determined cDNA sequence for clone | 63695180 R0670: H04 |
| SEQ ID NO: 877 | is the determined cDNA sequence for clone | 63695181 R0670: H05 |
| SEQ ID NO: 878 | is the determined cDNA sequence for clone | 63695182 R0670: H06 |
| SEQ ID NO: 879 | is the determined cDNA sequence for clone | 63695183 R0670: H07 |
| SEQ ID NO: 880 | is the determined cDNA sequence for clone | 63695184 R0670: H08 |
| SEQ ID NO: 881 | is the determined cDNA sequence for clone | 63695185 R0670: H09 |
| SEQ ID NO: 882 | is the determined cDNA sequence for clone | 63695186 R0670: H10 |
| SEQ ID NO: 883 | is the determined cDNA sequence for clone | 63695187 R0670: H11 |
| SEQ ID NO: 884 | is the determined cDNA sequence for clone | 63695653 R0671: A02 |
| SEQ ID NO: 885 | is the determined cDNA sequence for clone | 63695654 R0671: A03 |
| SEQ ID NO: 886 | is the determined cDNA sequence for clone | 63695655 R0671: A05 |
| SEQ ID NO: 887 | is the determined cDNA sequence for clone | 63695657 R0671: A07 |
| SEQ ID NO: 888 | is the determined cDNA sequence for clone | 63695659 R0671: A09 |
| SEQ ID NO: 889 | is the determined cDNA sequence for clone | 63695660 R0671: A10 |
| SEQ ID NO: 890 | is the determined cDNA sequence for clone | 63695661 R0671: A11 |
| SEQ ID NO: 891 | is the determined cDNA sequence for clone | 63695663 R0671: B01 |
| SEQ ID NO: 892 | is the determined cDNA sequence for clone | 63695664 R0671: B02 |
| SEQ ID NO: 893 | is the determined cDNA sequence for clone | 63695665 R0671: B03 |
| SEQ ID NO: 894 | is the determined cDNA sequence for clone | 63695666 R0671: B04 |
| SEQ ID NO: 895 | is the determined cDNA sequence for clone | 63695667 R0671: B05 |
| SEQ ID NO: 896 | is the determined cDNA sequence for clone | 63695668 R0671: B06 |
| SEQ ID NO: 897 | is the determined cDNA sequence for clone | 63695669 R0671: B07 |
| SEQ ID NO: 898 | is the determined cDNA sequence for clone | 63695670 R0671: B08 |
| SEQ ID NO: 899 | is the determined cDNA sequence for clone | 63695671 R0671: B09 |
| SEQ ID NO: 900 | is the determined cDNA sequence for clone | 63695672 R0671: B10 |
| SEQ ID NO: 901 | is the determined cDNA sequence for clone | 63695673 R0671: B11 |
| SEQ ID NO: 902 | is the determined cDNA sequence for clone | 63695675 R0671: C01 |
| SEQ ID NO: 903 | is the determined cDNA sequence for clone | 63695676 R0671: C02 |
| SEQ ID NO: 904 | is the determined cDNA sequence for clone | 63695678 R0671: C04 |
| SEQ ID NO: 905 | is the determined cDNA sequence for clone | 63695679 R0671: C05 |
| SEQ ID NO: 906 | is the determined cDNA sequence for clone | 63695680 R0671: C06 |
| SEQ ID NO: 907 | is the determined cDNA sequence for clone | 63695682 R0671: C08 |
| SEQ ID NO: 908 | is the determined cDNA sequence for clone | 63695683 R0671: C09 |
| SEQ ID NO: 909 | is the determined cDNA sequence for clone | 63695685 R0671: C11 |
| SEQ ID NO: 910 | is the determined cDNA sequence for clone | 63695686 R0671: C12 |
| SEQ ID NO: 911 | is the determined cDNA sequence for clone | 63695687 R0671: D01 |
| SEQ ID NO: 912 | is the determined cDNA sequence for clone | 63695688 R0671: D02 |
| SEQ ID NO: 913 | is the determined cDNA sequence for clone | 63695689 R0671: D03 |
| SEQ ID NO: 914 | is the determined cDNA sequence for clone | 63695690 R0671: D04 |
| SEQ ID NO: 915 | is the determined cDNA sequence for clone | 63695691 R0671: D05 |
| SEQ ID NO: 916 | is the determined cDNA sequence for clone | 63695692 R0671: D06 |
| SEQ ID NO: 917 | is the determined cDNA sequence for clone | 63695693 R0671: D07 |
| SEQ ID NO: 918 | is the determined cDNA sequence for clone | 63695694 R0671: D08 |
| SEQ ID NO: 919 | is the determined cDNA sequence for clone | 63695695 R0671: D09 |
| SEQ ID NO: 920 | is the determined cDNA sequence for clone | 63695696 R0671: D10 |
| SEQ ID NO: 921 | is the determined cDNA sequence for clone | 63695697 R0671: D11 |
| SEQ ID NO: 922 | is the determined cDNA sequence for clone | 63695698 R0671: D12 |
| SEQ ID NO: 923 | is the determined cDNA sequence for clone | 63695699 R0671: E01 |
| SEQ ID NO: 924 | is the determined cDNA sequence for clone | 63695700 R0671: E02 |
| SEQ ID NO: 925 | is the determined cDNA sequence for clone | 63695701 R0671: E03 |
| SEQ ID NO: 926 | is the determined cDNA sequence for clone | 63695702 R0671: E04 |
| SEQ ID NO: 927 | is the determined cDNA sequence for clone | 63695703 R0671: E05 |
| SEQ ID NO: 928 | is the determined cDNA sequence for clone | 63695704 R0671: E06 |
| SEQ ID NO: 929 | is the determined cDNA sequence for clone | 63695705 R0671: E07 |
| SEQ ID NO: 930 | is the determined cDNA sequence for clone | 63695706 R0671: E08 |
| SEQ ID NO: 931 | is the determined cDNA sequence for clone | 63695708 R0671: E10 |
| SEQ ID NO: 932 | is the determined cDNA sequence for clone | 63695710 R0671: E12 |
| SEQ ID NO: 933 | is the determined cDNA sequence for clone | 63695711 R0671: F01 |
| SEQ ID NO: 934 | is the determined cDNA sequence for clone | 63695712 R0671: F02 |
| SEQ ID NO: 935 | is the determined cDNA sequence for clone | 63695713 R0671: F03 |
| SEQ ID NO: 936 | is the determined cDNA sequence for clone | 63695715 R0671: F05 |
| SEQ ID NO: 937 | is the determined cDNA sequence for clone | 63695716 R0671: F06 |
| SEQ ID NO: 938 | is the determined cDNA sequence for clone | 63695717 R0671: F07 |
| SEQ ID NO: 939 | is the determined cDNA sequence for clone | 63695718 R0671: F08 |
| SEQ ID NO: 940 | is the determined cDNA sequence for clone | 63695719 R0671: F09 |
| SEQ ID NO: 941 | is the determined cDNA sequence for clone | 63695720 R0671: F10 |
| SEQ ID NO: 942 | is the determined cDNA sequence for clone | 63695721 R0671: F11 |
| SEQ ID NO: 943 | is the determined cDNA sequence for clone | 63695722 R0671: F12 |
| SEQ ID NO: 944 | is the determined cDNA sequence for clone | 63695723 R0671: G01 |
| SEQ ID NO: 945 | is the determined cDNA sequence for clone | 63695724 R0671: G02 |
| SEQ ID NO: 946 | is the determined cDNA sequence for clone | 63695725 R0671: G03 |
| SEQ ID NO: 947 | is the determined cDNA sequence for clone | 63695727 R0671: G05 |
| SEQ ID NO: 948 | is the determined cDNA sequence for clone | 63695728 R0671: G06 |
| SEQ ID NO: 949 | is the determined cDNA sequence for clone | 63695729 R0671: G07 |
| SEQ ID NO: 950 | is the determined cDNA sequence for clone | 63695730 R0671: G08 |
| SEQ ID NO: 951 | is the determined cDNA sequence for clone | 63695733 R0671: G11 |
| SEQ ID NO: 952 | is the determined cDNA sequence for clone | 63695734 R0671: G12 |
| SEQ ID NO: 953 | is the determined cDNA sequence for clone | 63695735 R0671: H01 |
| SEQ ID NO: 954 | is the determined cDNA sequence for clone | 63695736 R0671: H02 |
| SEQ ID NO: 955 | is the determined cDNA sequence for clone | 63695737 R0671: H03 |
| SEQ ID NO: 956 | is the determined cDNA sequence for clone | 63695738 R0671: H04 |
| SEQ ID NO: 957 | is the determined cDNA sequence for clone | 63695739 R0671: H05 |
| SEQ ID NO: 958 | is the determined cDNA sequence for clone | 63695740 R0671: H06 |
| SEQ ID NO: 959 | is the determined cDNA sequence for clone | 63695741 R0671: H07 |
| SEQ ID NO: 960 | is the determined cDNA sequence for clone | 63695742 R0671: H08 |
| SEQ ID NO: 961 | is the determined cDNA sequence for clone | 63695743 R0671: H09 |
| SEQ ID NO: 962 | is the determined cDNA sequence for clone | 63695744 R0671: H10 |
| SEQ ID NO: 963 | is the determined cDNA sequence for clone | 63695745 R0671: H11 |
| SEQ ID NO: 964 | is the determined cDNA sequence for clone | 63695002 R0672: A02 |
| SEQ ID NO: 965 | is the determined cDNA sequence for clone | 63695003 R0672: A03 |
| SEQ ID NO: 966 | is the determined cDNA sequence for clone | 63695004 R0672: A05 |
| SEQ ID NO: 967 | is the determined cDNA sequence for clone | 63695005 R0672: A06 |
| SEQ ID NO: 968 | is the determined cDNA sequence for clone | 63695007 R0672: A08 |
| SEQ ID NO: 969 | is the determined cDNA sequence for clone | 63695008 R0672: A09 |
| SEQ ID NO: 970 | is the determined cDNA sequence for clone | 63695009 R0672: A10 |
| SEQ ID NO: 971 | is the determined cDNA sequence for clone | 63695010 R0672: A11 |
| SEQ ID NO: 972 | is the determined cDNA sequence for clone | 63695011 R0672: A12 |
| SEQ ID NO: 973 | is the determined cDNA sequence for clone | 63695012 R0672: B01 |
| SEQ ID NO: 974 | is the determined cDNA sequence for clone | 63695013 R0672: B02 |
| SEQ ID NO: 975 | is the determined cDNA sequence for clone | 63695015 R0672: B04 |
| SEQ ID NO: 976 | is the determined cDNA sequence for clone | 63695016 R0672: B05 |
| SEQ ID NO: 977 | is the determined cDNA sequence for clone | 63695017 R0672: B06 |
| SEQ ID NO: 978 | is the determined cDNA sequence for clone | 63695018 R0672: B07 |
| SEQ ID NO: 979 | is the determined cDNA sequence for clone | 63695019 R0672: B08 |
| SEQ ID NO: 980 | is the determined cDNA sequence for clone | 63695020 R0672: B09 |
| SEQ ID NO: 981 | is the determined cDNA sequence for clone | 63695021 R0672: B10 |
| SEQ ID NO: 982 | is the determined cDNA sequence for clone | 63695022 R0672: B11 |
| SEQ ID NO: 983 | is the determined cDNA sequence for clone | 63695023 R0672: B12 |
| SEQ ID NO: 984 | is the determined cDNA sequence for clone | 63695024 R0672: C01 |
| SEQ ID NO: 985 | is the determined cDNA sequence for clone | 63695025 R0672: C02 |
| SEQ ID NO: 986 | is the determined cDNA sequence for clone | 63695026 R0672: C03 |
| SEQ ID NO: 987 | is the determined cDNA sequence for clone | 63695027 R0672: C04 |
| SEQ ID NO: 988 | is the determined cDNA sequence for clone | 63695028 R0672: C05 |
| SEQ ID NO: 989 | is the determined cDNA sequence for clone | 63695029 R0672: C06 |
| SEQ ID NO: 990 | is the determined cDNA sequence for clone | 63695030 R0672: C07 |
| SEQ ID NO: 991 | is the determined cDNA sequence for clone | 63695031 R0672: C08 |
| SEQ ID NO: 992 | is the determined cDNA sequence for clone | 63695032 R0672: C09 |
| SEQ ID NO: 993 | is the determined cDNA sequence for clone | 63695033 R0672: C10 |
| SEQ ID NO: 994 | is the determined cDNA sequence for clone | 63695034 R0672: C11 |
| SEQ ID NO: 995 | is the determined cDNA sequence for clone | 63695035 R0672: C12 |
| SEQ ID NO: 996 | is the determined cDNA sequence for clone | 63695036 R0672: D01 |
| SEQ ID NO: 997 | is the determined cDNA sequence for clone | 63695037 R0672: D02 |
| SEQ ID NO: 998 | is the determined cDNA sequence for clone | 63695038 R0672: D03 |
| SEQ ID NO: 999 | is the determined cDNA sequence for clone | 63695039 R0672: D04 |
| SEQ ID NO: 1000 | is the determined cDNA sequence for clone | 63695040 R0672: D05 |
| SEQ ID NO: 1001 | is the determined cDNA sequence for clone | 63695043 R0672: D08 |
| SEQ ID NO: 1002 | is the determined cDNA sequence for clone | 63695044 R0672: D09 |
| SEQ ID NO: 1003 | is the determined cDNA sequence for clone | 63695045 R0672: D10 |
| SEQ ID NO: 1004 | is the determined cDNA sequence for clone | 63695046 R0672: D11 |
| SEQ ID NO: 1005 | is the determined cDNA sequence for clone | 63695047 R0672: D12 |
| SEQ ID NO: 1006 | is the determined cDNA sequence for clone | 63695048 R0672: E01 |
| SEQ ID NO: 1007 | is the determined cDNA sequence for clone | 63695049 R0672: E02 |
| SEQ ID NO: 1008 | is the determined cDNA sequence for clone | 63695050 R0672: E03 |
| SEQ ID NO: 1009 | is the determined cDNA sequence for clone | 63695051 R0672: E04 |
| SEQ ID NO: 1010 | is the determined cDNA sequence for clone | 63695052 R0672: E05 |
| SEQ ID NO: 1011 | is the determined cDNA sequence for clone | 63695053 R0672: E06 |
| SEQ ID NO: 1012 | is the determined cDNA sequence for clone | 63695054 R0672: E07 |
| SEQ ID NO: 1013 | is the determined cDNA sequence for clone | 63695055 R0672: E08 |
| SEQ ID NO: 1014 | is the determined cDNA sequence for clone | 63695056 R0672: E09 |
| SEQ ID NO: 1015 | is the determined cDNA sequence for clone | 63695057 R0672: E10 |
| SEQ ID NO: 1016 | is the determined cDNA sequence for clone | 63695058 R0672: E11 |
| SEQ ID NO: 1017 | is the determined cDNA sequence for clone | 63695059 R0672: E12 |
| SEQ ID NO: 1018 | is the determined cDNA sequence for clone | 63695060 R0672: F01 |
| SEQ ID NO: 1019 | is the determined cDNA sequence for clone | 63695061 R0672: F02 |
| SEQ ID NO: 1020 | is the determined cDNA sequence for clone | 63695062 R0672: F03 |
| SEQ ID NO: 1021 | is the determined cDNA sequence for clone | 63695063 R0672: F04 |
| SEQ ID NO: 1022 | is the determined cDNA sequence for clone | 63695064 R0672: F05 |
| SEQ ID NO: 1023 | is the determined cDNA sequence for clone | 63695065 R0672: F06 |
| SEQ ID NO: 1024 | is the determined cDNA sequence for clone | 63695066 R0672: F07 |
| SEQ ID NO: 1025 | is the determined cDNA sequence for clone | 63695068 R0672: F09 |
| SEQ ID NO: 1026 | is the determined cDNA sequence for clone | 63695069 R0672: F10 |
| SEQ ID NO: 1027 | is the determined cDNA sequence for clone | 63695070 R0672: F11 |
| SEQ ID NO: 1028 | is the determined cDNA sequence for clone | 63695071 R0672: F12 |
| SEQ ID NO: 1029 | is the determined cDNA sequence for clone | 63695072 R0672: G01 |
| SEQ ID NO: 1030 | is the determined cDNA sequence for clone | 63695073 R0672: G02 |
| SEQ ID NO: 1031 | is the determined cDNA sequence for clone | 63695074 R0672: G03 |
| SEQ ID NO: 1032 | is the determined cDNA sequence for clone | 63695075 R0672: G04 |
| SEQ ID NO: 1033 | is the determined cDNA sequence for clone | 63695076 R0672: G05 |
| SEQ ID NO: 1034 | is the determined cDNA sequence for clone | 63695077 R0672: G06 |
| SEQ ID NO: 1035 | is the determined cDNA sequence for clone | 63695078 R0672: G07 |
| SEQ ID NO: 1036 | is the determined cDNA sequence for clone | 63695079 R0672: G08 |
| SEQ ID NO: 1037 | is the determined cDNA sequence for clone | 63695080 R0672: G09 |
| SEQ ID NO: 1038 | is the determined cDNA sequence for clone | 63695081 R0672: G10 |
| SEQ ID NO: 1039 | is the determined cDNA sequence for clone | 63695082 R0672: G11 |
| SEQ ID NO: 1040 | is the determined cDNA sequence for clone | 63695083 R0672: G12 |
| SEQ ID NO: 1041 | is the determined cDNA sequence for clone | 63695085 R0672: H02 |
| SEQ ID NO: 1042 | is the determined cDNA sequence for clone | 63695086 R0672: H03 |
| SEQ ID NO: 1043 | is the determined cDNA sequence for clone | 63695087 R0672: H04 |
| SEQ ID NO: 1044 | is the determined cDNA sequence for clone | 63695088 R0672: H05 |
| SEQ ID NO: 1045 | is the determined cDNA sequence for clone | 63695089 R0672: H06 |
| SEQ ID NO: 1046 | is the determined cDNA sequence for clone | 63695090 R0672: H07 |
| SEQ ID NO: 1047 | is the determined cDNA sequence for clone | 63695091 R0672: H08 |
| SEQ ID NO: 1048 | is the determined cDNA sequence for clone | 63695092 R0672: H09 |
| SEQ ID NO: 1049 | is the determined cDNA sequence for clone | 63695093 R0672: H10 |
| SEQ ID NO: 1050 | is the determined cDNA sequence for clone | 63695094 R0672: H11 |
| SEQ ID NO: 1051 | is the determined cDNA sequence for clone | 63695282 R0673: A03 |
| SEQ ID NO: 1052 | is the determined cDNA sequence for clone | 63695284 R0673: A06 |
| SEQ ID NO: 1053 | is the determined cDNA sequence for clone | 63695285 R0673: A07 |
| SEQ ID NO: 1054 | is the determined cDNA sequence for clone | 63695286 R0673: A08 |
| SEQ ID NO: 1055 | is the determined cDNA sequence for clone | 63695287 R0673: A09 |
| SEQ ID NO: 1056 | is the determined cDNA sequence for clone | 63695289 R0673: A11 |
| SEQ ID NO: 1057 | is the determined cDNA sequence for clone | 63695290 R0673: A12 |
| SEQ ID NO: 1058 | is the determined cDNA sequence for clone | 63695291 R0673: B01 |
| SEQ ID NO: 1059 | is the determined cDNA sequence for clone | 63695292 R0673: B02 |
| SEQ ID NO: 1060 | is the determined cDNA sequence for clone | 63695294 R0673: B04 |
| SEQ ID NO: 1061 | is the determined cDNA sequence for clone | 63695295 R0673: B05 |
| SEQ ID NO: 1062 | is the determined cDNA sequence for clone | 63695296 R0673: B06 |
| SEQ ID NO: 1063 | is the determined cDNA sequence for clone | 63695297 R0673: B07 |
| SEQ ID NO: 1064 | is the determined cDNA sequence for clone | 63695298 R0673: B08 |
| SEQ ID NO: 1065 | is the determined cDNA sequence for clone | 63695301 R0673: B11 |
| SEQ ID NO: 1066 | is the determined cDNA sequence for clone | 63695303 R0673: C01 |
| SEQ ID NO: 1067 | is the determined cDNA sequence for clone | 63695304 R0673: C02 |
| SEQ ID NO: 1068 | is the determined cDNA sequence for clone | 63695305 R0673: C03 |
| SEQ ID NO: 1069 | is the determined cDNA sequence for clone | 63695306 R0673: C04 |
| SEQ ID NO: 1070 | is the determined cDNA sequence for clone | 63695307 R0673: C05 |
| SEQ ID NO: 1071 | is the determined cDNA sequence for clone | 63695308 R0673: C06 |
| SEQ ID NO: 1072 | is the determined cDNA sequence for clone | 63695310 R0673: C08 |
| SEQ ID NO: 1073 | is the determined cDNA sequence for clone | 63695311 R0673: C09 |
| SEQ ID NO: 1074 | is the determined cDNA sequence for clone | 63695312 R0673: C10 |
| SEQ ID NO: 1075 | is the determined cDNA sequence for clone | 63695313 R0673: C11 |
| SEQ ID NO: 1076 | is the determined cDNA sequence for clone | 63695314 R0673: C12 |
| SEQ ID NO: 1077 | is the determined cDNA sequence for clone | 63695315 R0673: D01 |
| SEQ ID NO: 1078 | is the determined cDNA sequence for clone | 63695316 R0673: D02 |
| SEQ ID NO: 1079 | is the determined cDNA sequence for clone | 63695317 R0673: D03 |
| SEQ ID NO: 1080 | is the determined cDNA sequence for clone | 63695318 R0673: D04 |
| SEQ ID NO: 1081 | is the determined cDNA sequence for clone | 63695319 R0673: D05 |
| SEQ ID NO: 1082 | is the determined cDNA sequence for clone | 63695320 R0673: D06 |
| SEQ ID NO: 1083 | is the determined cDNA sequence for clone | 63695321 R0673: D07 |
| SEQ ID NO: 1084 | is the determined cDNA sequence for clone | 63695323 R0673: D09 |
| SEQ ID NO: 1085 | is the determined cDNA sequence for clone | 63695324 R0673: D10 |
| SEQ ID NO: 1086 | is the determined cDNA sequence for clone | 63695325 R0673: D11 |
| SEQ ID NO: 1087 | is the determined cDNA sequence for clone | 63695326 R0673: D12 |
| SEQ ID NO: 1088 | is the determined cDNA sequence for clone | 63695327 R0673: E01 |
| SEQ ID NO: 1089 | is the determined cDNA sequence for clone | 63695328 R0673: E02 |
| SEQ ID NO: 1090 | is the determined cDNA sequence for clone | 63695329 R0673: E03 |
| SEQ ID NO: 1091 | is the determined cDNA sequence for clone | 63695330 R0673: E04 |
| SEQ ID NO: 1092 | is the determined cDNA sequence for clone | 63695331 R0673: E05 |
| SEQ ID NO: 1093 | is the determined cDNA sequence for clone | 63695333 R0673: E07 |
| SEQ ID NO: 1094 | is the determined cDNA sequence for clone | 63695334 R0673: E08 |
| SEQ ID NO: 1095 | is the determined cDNA sequence for clone | 63695335 R0673: E09 |
| SEQ ID NO: 1096 | is the determined cDNA sequence for clone | 63695337 R0673: E11 |
| SEQ ID NO: 1097 | is the determined cDNA sequence for clone | 63695338 R0673: E12 |
| SEQ ID NO: 1098 | is the determined cDNA sequence for clone | 63695339 R0673: F01 |
| SEQ ID NO: 1099 | is the determined cDNA sequence for clone | 63695341 R0673: F03 |
| SEQ ID NO: 1100 | is the determined cDNA sequence for clone | 63695342 R0673: F04 |
| SEQ ID NO: 1101 | is the determined cDNA sequence for clone | 63695344 R0673: F06 |
| SEQ ID NO: 1102 | is the determined cDNA sequence for clone | 63695346 R0673: F08 |
| SEQ ID NO: 1103 | is the determined cDNA sequence for clone | 63695347 R0673: F09 |
| SEQ ID NO: 1104 | is the determined cDNA sequence for clone | 63695348 R0673: F10 |
| SEQ ID NO: 1105 | is the determined cDNA sequence for clone | 63695349 R0673: F11 |
| SEQ ID NO: 1106 | is the determined cDNA sequence for clone | 63695350 R0673: F12 |
| SEQ ID NO: 1107 | is the determined cDNA sequence for clone | 63695351 R0673: G01 |
| SEQ ID NO: 1108 | is the determined cDNA sequence for clone | 63695352 R0673: G02 |
| SEQ ID NO: 1109 | is the determined cDNA sequence for clone | 63695353 R0673: G03 |
| SEQ ID NO: 1110 | is the determined cDNA sequence for clone | 63695354 R0673: G04 |
| SEQ ID NO: 1111 | is the determined cDNA sequence for clone | 63695356 R0673: G06 |
| SEQ ID NO: 1112 | is the determined cDNA sequence for clone | 63695357 R0673: G07 |
| SEQ ID NO: 1113 | is the determined cDNA sequence for clone | 63695358 R0673: G08 |
| SEQ ID NO: 1114 | is the determined cDNA sequence for clone | 63695359 R0673: G09 |
| SEQ ID NO: 1115 | is the determined cDNA sequence for clone | 63695361 R0673: G11 |
| SEQ ID NO: 1116 | is the determined cDNA sequence for clone | 63695363 R0673: H01 |
| SEQ ID NO: 1117 | is the determined cDNA sequence for clone | 63695364 R0673: H02 |
| SEQ ID NO: 1118 | is the determined cDNA sequence for clone | 63695366 R0673: H04 |
| SEQ ID NO: 1119 | is the determined cDNA sequence for clone | 63695367 R0673: H05 |
| SEQ ID NO: 1120 | is the determined cDNA sequence for clone | 63695368 R0673: H06 |
| SEQ ID NO: 1121 | is the determined cDNA sequence for clone | 63695369 R0673: H07 |
| SEQ ID NO: 1122 | is the determined cDNA sequence for clone | 63695370 R0673: H08 |
| SEQ ID NO: 1123 | is the determined cDNA sequence for clone | 63695371 R0673: H09 |
| SEQ ID NO: 1124 | is the determined cDNA sequence for clone | 63695372 R0673: H10 |
| SEQ ID NO: 1125 | is the determined cDNA sequence for clone | 63695373 R0673: H11 |
| SEQ ID NO: 1126 | is the determined cDNA sequence for clone | 63695188 R0674: A02 |
| SEQ ID NO: 1127 | is the determined cDNA sequence for clone | 63695189 R0674: A03 |
| SEQ ID NO: 1128 | is the determined cDNA sequence for clone | 63695190 R0674: A05 |
| SEQ ID NO: 1129 | is the determined cDNA sequence for clone | 63695191 R0674: A06 |
| SEQ ID NO: 1130 | is the determined cDNA sequence for clone | 63695192 R0674: A07 |
| SEQ ID NO: 1131 | is the determined cDNA sequence for clone | 63695194 R0674: A09 |
| SEQ ID NO: 1132 | is the determined cDNA sequence for clone | 63695196 R0674: A11 |
| SEQ ID NO: 1133 | is the determined cDNA sequence for clone | 63695197 R0674: A12 |
| SEQ ID NO: 1134 | is the determined cDNA sequence for clone | 63695198 R0674: B01 |
| SEQ ID NO: 1135 | is the determined cDNA sequence for clone | 63695199 R0674: B02 |
| SEQ ID NO: 1136 | is the determined cDNA sequence for clone | 63695200 R0674: B03 |
| SEQ ID NO: 1137 | is the determined cDNA sequence for clone | 63695202 R0674: B05 |
| SEQ ID NO: 1138 | is the determined cDNA sequence for clone | 63695203 R0674: B06 |
| SEQ ID NO: 1139 | is the determined cDNA sequence for clone | 63695205 R0674: B08 |
| SEQ ID NO: 1140 | is the determined cDNA sequence for clone | 63695206 R0674: B09 |
| SEQ ID NO: 1141 | is the determined cDNA sequence for clone | 63695207 R0674: B10 |
| SEQ ID NO: 1142 | is the determined cDNA sequence for clone | 63695208 R0674: B11 |
| SEQ ID NO: 1143 | is the determined cDNA sequence for clone | 63695209 R0674: B12 |
| SEQ ID NO: 1144 | is the determined cDNA sequence for clone | 63695210 R0674: C01 |
| SEQ ID NO: 1145 | is the determined cDNA sequence for clone | 63695212 R0674: C03 |
| SEQ ID NO: 1146 | is the determined cDNA sequence for clone | 63695213 R0674: C04 |
| SEQ ID NO: 1147 | is the determined cDNA sequence for clone | 63695214 R0674: C05 |
| SEQ ID NO: 1148 | is the determined cDNA sequence for clone | 63695216 R0674: C07 |
| SEQ ID NO: 1149 | is the determined cDNA sequence for clone | 63695218 R0674: C09 |
| SEQ ID NO: 1150 | is the determined cDNA sequence for clone | 63695220 R0674: C11 |
| SEQ ID NO: 1151 | is the determined cDNA sequence for clone | 63695221 R0674: C12 |
| SEQ ID NO: 1152 | is the determined cDNA sequence for clone | 63695223 R0674: D02 |
| SEQ ID NO: 1153 | is the determined cDNA sequence for clone | 63695224 R0674: D03 |
| SEQ ID NO: 1154 | is the determined cDNA sequence for clone | 63695225 R0674: D04 |
| SEQ ID NO: 1155 | is the determined cDNA sequence for clone | 63695226 R0674: D05 |
| SEQ ID NO: 1156 | is the determined cDNA sequence for clone | 63695227 R0674: D06 |
| SEQ ID NO: 1157 | is the determined cDNA sequence for clone | 63695228 R0674: D07 |
| SEQ ID NO: 1158 | is the determined cDNA sequence for clone | 63695234 R0674: E01 |
| SEQ ID NO: 1159 | is the determined cDNA sequence for clone | 63695236 R0674: E03 |
| SEQ ID NO: 1160 | is the determined cDNA sequence for clone | 63695237 R0674: E04 |
| SEQ ID NO: 1161 | is the determined cDNA sequence for clone | 63695238 R0674: E05 |
| SEQ ID NO: 1162 | is the determined cDNA sequence for clone | 63695241 R0674: E08 |
| SEQ ID NO: 1163 | is the determined cDNA sequence for clone | 63695244 R0674: E11 |
| SEQ ID NO: 1164 | is the determined cDNA sequence for clone | 63695247 R0674: F02 |
| SEQ ID NO: 1165 | is the determined cDNA sequence for clone | 63695248 R0674: F03 |
| SEQ ID NO: 1166 | is the determined cDNA sequence for clone | 63695249 R0674: F04 |
| SEQ ID NO: 1167 | is the determined cDNA sequence for clone | 63695250 R0674: F05 |
| SEQ ID NO: 1168 | is the determined cDNA sequence for clone | 63695251 R0674: F06 |
| SEQ ID NO: 1169 | is the determined cDNA sequence for clone | 63695252 R0674: F07 |
| SEQ ID NO: 1170 | is the determined cDNA sequence for clone | 63695255 R0674: F10 |
| SEQ ID NO: 1171 | is the determined cDNA sequence for clone | 63695256 R0674: F11 |
| SEQ ID NO: 1172 | is the determined cDNA sequence for clone | 63695257 R0674: F12 |
| SEQ ID NO: 1173 | is the determined cDNA sequence for clone | 63695261 R0674: G04 |
| SEQ ID NO: 1174 | is the determined cDNA sequence for clone | 63695262 R0674: G05 |
| SEQ ID NO: 1175 | is the determined cDNA sequence for clone | 63695263 R0674: G06 |
| SEQ ID NO: 1176 | is the determined cDNA sequence for clone | 63695264 R0674: G07 |
| SEQ ID NO: 1177 | is the determined cDNA sequence for clone | 63695265 R0674: G08 |
| SEQ ID NO: 1178 | is the determined cDNA sequence for clone | 63695266 R0674: G09 |
| SEQ ID NO: 1179 | is the determined cDNA sequence for clone | 63695267 R0674: G10 |
| SEQ ID NO: 1180 | is the determined cDNA sequence for clone | 63695268 R0674: G11 |
| SEQ ID NO: 1181 | is the determined cDNA sequence for clone | 63695270 R0674: H01 |
| SEQ ID NO: 1182 | is the determined cDNA sequence for clone | 63695271 R0674: H02 |
| SEQ ID NO: 1183 | is the determined cDNA sequence for clone | 63695272 R0674: H03 |
| SEQ ID NO: 1184 | is the determined cDNA sequence for clone | 63695273 R0674: H04 |
| SEQ ID NO: 1185 | is the determined cDNA sequence for clone | 63695274 R0674: H05 |
| SEQ ID NO: 1186 | is the determined cDNA sequence for clone | 63695275 R0674: H06 |
| SEQ ID NO: 1187 | is the determined cDNA sequence for clone | 63695276 R0674: H07 |
| SEQ ID NO: 1188 | is the determined cDNA sequence for clone | 63695278 R0674: H09 |
| SEQ ID NO: 1189 | is the determined cDNA sequence for clone | 63695279 R0674: H10 |
| SEQ ID NO: 1190 | is the determined cDNA sequence for clone | 63695280 R0674: H11 |
| SEQ ID NO: 1191 | is the determined cDNA sequence for clone | 63694910 R0675: A03 |
| SEQ ID NO: 1192 | is the determined cDNA sequence for clone | 63694911 R0675: A05 |
| SEQ ID NO: 1193 | is the determined cDNA sequence for clone | 63694912 R0675: A06 |
| SEQ ID NO: 1194 | is the determined cDNA sequence for clone | 63694913 R0675: A07 |
| SEQ ID NO: 1195 | is the determined cDNA sequence for clone | 63694914 R0675: A08 |
| SEQ ID NO: 1196 | is the determined cDNA sequence for clone | 63694915 R0675: A09 |
| SEQ ID NO: 1197 | is the determined cDNA sequence for clone | 63694916 R0675: A10 |
| SEQ ID NO: 1198 | is the determined cDNA sequence for clone | 63694917 R0675: A11 |
| SEQ ID NO: 1199 | is the determined cDNA sequence for clone | 63694918 R0675: A12 |
| SEQ ID NO: 1200 | is the determined cDNA sequence for clone | 63694919 R0675: B01 |
| SEQ ID NO: 1201 | is the determined cDNA sequence for clone | 63694920 R0675: B02 |
| SEQ ID NO: 1202 | is the determined cDNA sequence for clone | 63694921 R0675: B03 |
| SEQ ID NO: 1203 | is the determined cDNA sequence for clone | 63694922 R0675: B04 |
| SEQ ID NO: 1204 | is the determined cDNA sequence for clone | 63694923 R0675: B05 |
| SEQ ID NO: 1205 | is the determined cDNA sequence for clone | 63694924 R0675: B06 |
| SEQ ID NO: 1206 | is the determined cDNA sequence for clone | 63694925 R0675: B07 |
| SEQ ID NO: 1207 | is the determined cDNA sequence for clone | 63694926 R0675: B08 |
| SEQ ID NO: 1208 | is the determined cDNA sequence for clone | 63694927 R0675: B09 |
| SEQ ID NO: 1209 | is the determined cDNA sequence for clone | 63694928 R0675: B10 |
| SEQ ID NO: 1210 | is the determined cDNA sequence for clone | 63694929 R0675: B11 |
| SEQ ID NO: 1211 | is the determined cDNA sequence for clone | 63694930 R0675: B12 |
| SEQ ID NO: 1212 | is the determined cDNA sequence for clone | 63694931 R0675: C01 |
| SEQ ID NO: 1213 | is the determined cDNA sequence for clone | 63694932 R0675: C02 |
| SEQ ID NO: 1214 | is the determined cDNA sequence for clone | 63694934 R0675: C04 |
| SEQ ID NO: 1215 | is the determined cDNA sequence for clone | 63694935 R0675: C05 |
| SEQ ID NO: 1216 | is the determined cDNA sequence for clone | 63694936 R0675: C06 |
| SEQ ID NO: 1217 | is the determined cDNA sequence for clone | 63694937 R0675: C07 |
| SEQ ID NO: 1218 | is the determined cDNA sequence for clone | 63694938 R0675: C08 |
| SEQ ID NO: 1219 | is the determined cDNA sequence for clone | 63694939 R0675: C09 |
| SEQ ID NO: 1220 | is the determined cDNA sequence for clone | 63694940 R0675: C10 |
| SEQ ID NO: 1221 | is the determined cDNA sequence for clone | 63694941 R0675: C11 |
| SEQ ID NO: 1222 | is the determined cDNA sequence for clone | 63694943 R0675: D01 |
| SEQ ID NO: 1223 | is the determined cDNA sequence for clone | 63694944 R0675: D02 |
| SEQ ID NO: 1224 | is the determined cDNA sequence for clone | 63694946 R0675: D04 |
| SEQ ID NO: 1225 | is the determined cDNA sequence for clone | 63694947 R0675: D05 |
| SEQ ID NO: 1226 | is the determined cDNA sequence for clone | 63694948 R0675: D06 |
| SEQ ID NO: 1227 | is the determined cDNA sequence for clone | 63694949 R0675: D07 |
| SEQ ID NO: 1228 | is the determined cDNA sequence for clone | 63694950 R0675: D08 |
| SEQ ID NO: 1229 | is the determined cDNA sequence for clone | 63694952 R0675: D10 |
| SEQ ID NO: 1230 | is the determined cDNA sequence for clone | 63694953 R0675: D11 |
| SEQ ID NO: 1231 | is the determined cDNA sequence for clone | 63694954 R0675: D12 |
| SEQ ID NO: 1232 | is the determined cDNA sequence for clone | 63694955 R0675: E01 |
| SEQ ID NO: 1233 | is the determined cDNA sequence for clone | 63694958 R0675: E04 |
| SEQ ID NO: 1234 | is the determined cDNA sequence for clone | 63694959 R0675: E05 |
| SEQ ID NO: 1235 | is the determined cDNA sequence for clone | 63694960 R0675: E06 |
| SEQ ID NO: 1236 | is the determined cDNA sequence for clone | 63694961 R0675: E07 |
| SEQ ID NO: 1237 | is the determined cDNA sequence for clone | 63694962 R0675: E08 |
| SEQ ID NO: 1238 | is the determined cDNA sequence for clone | 63694963 R0675: E09 |
| SEQ ID NO: 1239 | is the determined cDNA sequence for clone | 63694964 R0675: E10 |
| SEQ ID NO: 1240 | is the determined cDNA sequence for clone | 63694966 R0675: E12 |
| SEQ ID NO: 1241 | is the determined cDNA sequence for clone | 63694967 R0675: F01 |
| SEQ ID NO: 1242 | is the determined cDNA sequence for clone | 63694968 R0675: F02 |
| SEQ ID NO: 1243 | is the determined cDNA sequence for clone | 63694969 R0675: F03 |
| SEQ ID NO: 1244 | is the determined cDNA sequence for clone | 63694970 R0675: F04 |
| SEQ ID NO: 1245 | is the determined cDNA sequence for clone | 63694971 R0675: F05 |
| SEQ ID NO: 1246 | is the determined cDNA sequence for clone | 63694972 R0675: F06 |
| SEQ ID NO: 1247 | is the determined cDNA sequence for clone | 63694973 R0675: F07 |
| SEQ ID NO: 1248 | is the determined cDNA sequence for clone | 63694974 R0675: F08 |
| SEQ ID NO: 1249 | is the determined cDNA sequence for clone | 63694975 R0675: F09 |
| SEQ ID NO: 1250 | is the determined cDNA sequence for clone | 63694976 R0675: F10 |
| SEQ ID NO: 1251 | is the determined cDNA sequence for clone | 63694977 R0675: F11 |
| SEQ ID NO: 1252 | is the determined cDNA sequence for clone | 63694978 R0675: F12 |
| SEQ ID NO: 1253 | is the determined cDNA sequence for clone | 63694979 R0675: G01 |
| SEQ ID NO: 1254 | is the determined cDNA sequence for clone | 63694980 R0675: G02 |
| SEQ ID NO: 1255 | is the determined cDNA sequence for clone | 63694981 R0675: G03 |
| SEQ ID NO: 1256 | is the determined cDNA sequence for clone | 63694982 R0675: G04 |
| SEQ ID NO: 1257 | is the determined cDNA sequence for clone | 63694983 R0675: G05 |
| SEQ ID NO: 1258 | is the determined cDNA sequence for clone | 63694984 R0675: G06 |
| SEQ ID NO: 1259 | is the determined cDNA sequence for clone | 63694985 R0675: G07 |
| SEQ ID NO: 1260 | is the determined cDNA sequence for clone | 63694986 R0675: G08 |
| SEQ ID NO: 1261 | is the determined cDNA sequence for clone | 63694987 R0675: G09 |
| SEQ ID NO: 1262 | is the determined cDNA sequence for clone | 63694988 R0675: G10 |
| SEQ ID NO: 1263 | is the determined cDNA sequence for clone | 63694990 R0675: G12 |
| SEQ ID NO: 1264 | is the determined cDNA sequence for clone | 63694991 R0675: H01 |
| SEQ ID NO: 1265 | is the determined cDNA sequence for clone | 63694992 R0675: H02 |
| SEQ ID NO: 1266 | is the determined cDNA sequence for clone | 63694993 R0675: H03 |
| SEQ ID NO: 1267 | is the determined cDNA sequence for clone | 63694995 R0675: H05 |
| SEQ ID NO: 1268 | is the determined cDNA sequence for clone | 63694996 R0675: H06 |
| SEQ ID NO: 1269 | is the determined cDNA sequence for clone | 63694997 R0675: H07 |
| SEQ ID NO: 1270 | is the determined cDNA sequence for clone | 63694999 R0675: H09 |
| SEQ ID NO: 1271 | is the determined cDNA sequence for clone | 63695000 R0675: H10 |
| SEQ ID NO: 1272 | is the determined cDNA sequence for clone | 63695746 R0676: A02 |
| SEQ ID NO: 1273 | is the determined cDNA sequence for clone | 63695747 R0676: A03 |
| SEQ ID NO: 1274 | is the determined cDNA sequence for clone | 63695748 R0676: A05 |
| SEQ ID NO: 1275 | is the determined cDNA sequence for clone | 63695749 R0676: A06 |
| SEQ ID NO: 1276 | is the determined cDNA sequence for clone | 63695750 R0676: A07 |
| SEQ ID NO: 1277 | is the determined cDNA sequence for clone | 63695751 R0676: A08 |
| SEQ ID NO: 1278 | is the determined cDNA sequence for clone | 63695752 R0676: A09 |
| SEQ ID NO: 1279 | is the determined cDNA sequence for clone | 63695754 R0676: A11 |
| SEQ ID NO: 1280 | is the determined cDNA sequence for clone | 63695755 R0676: A12 |
| SEQ ID NO: 1281 | is the determined cDNA sequence for clone | 63695756 R0676: B01 |
| SEQ ID NO: 1282 | is the determined cDNA sequence for clone | 63695758 R0676: B03 |
| SEQ ID NO: 1283 | is the determined cDNA sequence for clone | 63695759 R0676: B04 |
| SEQ ID NO: 1284 | is the determined cDNA sequence for clone | 63695760 R0676: B05 |
| SEQ ID NO: 1285 | is the determined cDNA sequence for clone | 63695762 R0676: B07 |
| SEQ ID NO: 1286 | is the determined cDNA sequence for clone | 63695764 R0676: B09 |
| SEQ ID NO: 1287 | is the determined cDNA sequence for clone | 63695766 R0676: B11 |
| SEQ ID NO: 1288 | is the determined cDNA sequence for clone | 63695769 R0676: C02 |
| SEQ ID NO: 1289 | is the determined cDNA sequence for clone | 63695770 R0676: C03 |
| SEQ ID NO: 1290 | is the determined cDNA sequence for clone | 63695771 R0676: C04 |
| SEQ ID NO: 1291 | is the determined cDNA sequence for clone | 63695772 R0676: C05 |
| SEQ ID NO: 1292 | is the determined cDNA sequence for clone | 63695773 R0676: C06 |
| SEQ ID NO: 1293 | is the determined cDNA sequence for clone | 63695774 R0676: C07 |
| SEQ ID NO: 1294 | is the determined cDNA sequence for clone | 63695775 R0676: C08 |
| SEQ ID NO: 1295 | is the determined cDNA sequence for clone | 63695777 R0676: C10 |
| SEQ ID NO: 1296 | is the determined cDNA sequence for clone | 63695778 R0676: C11 |
| SEQ ID NO: 1297 | is the determined cDNA sequence for clone | 63695779 R0676: C12 |
| SEQ ID NO: 1298 | is the determined cDNA sequence for clone | 63695780 R0676: D01 |
| SEQ ID NO: 1299 | is the determined cDNA sequence for clone | 63695782 R0676: D03 |
| SEQ ID NO: 1300 | is the determined cDNA sequence for clone | 63695784 R0676: D05 |
| SEQ ID NO: 1301 | is the determined cDNA sequence for clone | 63695786 R0676: D07 |
| SEQ ID NO: 1302 | is the determined cDNA sequence for clone | 63695787 R0676: D08 |
| SEQ ID NO: 1303 | is the determined cDNA sequence for clone | 63695788 R0676: D09 |
| SEQ ID NO: 1304 | is the determined cDNA sequence for clone | 63695790 R0676: D11 |
| SEQ ID NO: 1305 | is the determined cDNA sequence for clone | 63695791 R0676: D12 |
| SEQ ID NO: 1306 | is the determined cDNA sequence for clone | 63695792 R0676: E01 |
| SEQ ID NO: 1307 | is the determined cDNA sequence for clone | 63695793 R0676: E02 |
| SEQ ID NO: 1308 | is the determined cDNA sequence for clone | 63695794 R0676: E03 |
| SEQ ID NO: 1309 | is the determined cDNA sequence for clone | 63695796 R0676: E05 |
| SEQ ID NO: 1310 | is the determined cDNA sequence for clone | 63695797 R0676: E06 |
| SEQ ID NO: 1311 | is the determined cDNA sequence for clone | 63695798 R0676: E07 |
| SEQ ID NO: 1312 | is the determined cDNA sequence for clone | 63695803 R0676: E12 |
| SEQ ID NO: 1313 | is the determined cDNA sequence for clone | 63695804 R0676: F01 |
| SEQ ID NO: 1314 | is the determined cDNA sequence for clone | 63695806 R0676: F03 |
| SEQ ID NO: 1315 | is the determined cDNA sequence for clone | 63695807 R0676: F04 |
| SEQ ID NO: 1316 | is the determined cDNA sequence for clone | 63695808 R0676: F05 |
| SEQ ID NO: 1317 | is the determined cDNA sequence for clone | 63695809 R0676: F06 |
| SEQ ID NO: 1318 | is the determined cDNA sequence for clone | 63695810 R0676: F07 |
| SEQ ID NO: 1319 | is the determined cDNA sequence for clone | 63695811 R0676: F08 |
| SEQ ID NO: 1320 | is the determined cDNA sequence for clone | 63695812 R0676: F09 |
| SEQ ID NO: 1321 | is the determined cDNA sequence for clone | 63695813 R0676: F10 |
| SEQ ID NO: 1322 | is the determined cDNA sequence for clone | 63695814 R0676: F11 |
| SEQ ID NO: 1323 | is the determined cDNA sequence for clone | 63695815 R0676: F12 |
| SEQ ID NO: 1324 | is the determined cDNA sequence for clone | 63695816 R0676: G01 |
| SEQ ID NO: 1325 | is the determined cDNA sequence for clone | 63695817 R0676: G02 |
| SEQ ID NO: 1326 | is the determined cDNA sequence for clone | 63695818 R0676: G03 |
| SEQ ID NO: 1327 | is the determined cDNA sequence for clone | 63695820 R0676: G05 |
| SEQ ID NO: 1328 | is the determined cDNA sequence for clone | 63695822 R0676: G07 |
| SEQ ID NO: 1329 | is the determined cDNA sequence for clone | 63695823 R0676: G08 |
| SEQ ID NO: 1330 | is the determined cDNA sequence for clone | 63695824 R0676: G09 |
| SEQ ID NO: 1331 | is the determined cDNA sequence for clone | 63695825 R0676: G10 |
| SEQ ID NO: 1332 | is the determined cDNA sequence for clone | 63695826 R0676: G11 |
| SEQ ID NO: 1333 | is the determined cDNA sequence for clone | 63695827 R0676: G12 |
| SEQ ID NO: 1334 | is the determined cDNA sequence for clone | 63695828 R0676: H01 |
| SEQ ID NO: 1335 | is the determined cDNA sequence for clone | 63695829 R0676: H02 |
| SEQ ID NO: 1336 | is the determined cDNA sequence for clone | 63695830 R0676: H03 |
| SEQ ID NO: 1337 | is the determined cDNA sequence for clone | 63695831 R0676: H04 |
| SEQ ID NO: 1338 | is the determined cDNA sequence for clone | 63695832 R0676: H05 |
| SEQ ID NO: 1339 | is the determined cDNA sequence for clone | 63695833 R0676: H06 |
| SEQ ID NO: 1340 | is the determined cDNA sequence for clone | 63695834 R0676: H07 |
| SEQ ID NO: 1341 | is the determined cDNA sequence for clone | 63695835 R0676: H08 |
| SEQ ID NO: 1342 | is the determined cDNA sequence for clone | 63695836 R0676: H09 |
| SEQ ID NO: 1343 | is the determined cDNA sequence for clone | 63695837 R0676: H10 |
| SEQ ID NO: 1344 | is the determined cDNA sequence for clone | 63695838 R0676: H11 |
| SEQ ID NO: 1345 | is the determined cDNA sequence for clone | 63695374 R0677: A02 |
| SEQ ID NO: 1346 | is the determined cDNA sequence for clone | 63695375 R0677: A03 |
| SEQ ID NO: 1347 | is the determined cDNA sequence for clone | 63695376 R0677: A05 |
| SEQ ID NO: 1348 | is the determined cDNA sequence for clone | 63695378 R0677: A07 |
| SEQ ID NO: 1349 | is the determined cDNA sequence for clone | 63695379 R0677: A08 |
| SEQ ID NO: 1350 | is the determined cDNA sequence for clone | 63695380 R0677: A09 |
| SEQ ID NO: 1351 | is the determined cDNA sequence for clone | 63695381 R0677: A10 |
| SEQ ID NO: 1352 | is the determined cDNA sequence for clone | 63695382 R0677: A11 |
| SEQ ID NO: 1353 | is the determined cDNA sequence for clone | 63695383 R0677: A12 |
| SEQ ID NO: 1354 | is the determined cDNA sequence for clone | 63695384 R0677: B01 |
| SEQ ID NO: 1355 | is the determined cDNA sequence for clone | 63695386 R0677: B03 |
| SEQ ID NO: 1356 | is the determined cDNA sequence for clone | 63695387 R0677: B04 |
| SEQ ID NO: 1357 | is the determined cDNA sequence for clone | 63695388 R0677: B05 |
| SEQ ID NO: 1358 | is the determined cDNA sequence for clone | 63695389 R0677: B06 |
| SEQ ID NO: 1359 | is the determined cDNA sequence for clone | 63695390 R0677: B07 |
| SEQ ID NO: 1360 | is the determined cDNA sequence for clone | 63695391 R0677: B08 |
| SEQ ID NO: 1361 | is the determined cDNA sequence for clone | 63695392 R0677: B09 |
| SEQ ID NO: 1362 | is the determined cDNA sequence for clone | 63695393 R0677: B10 |
| SEQ ID NO: 1363 | is the determined cDNA sequence for clone | 63695394 R0677: B11 |
| SEQ ID NO: 1364 | is the determined cDNA sequence for clone | 63695395 R0677: B12 |
| SEQ ID NO: 1365 | is the determined cDNA sequence for clone | 63695397 R0677: C02 |
| SEQ ID NO: 1366 | is the determined cDNA sequence for clone | 63695398 R0677: C03 |
| SEQ ID NO: 1367 | is the determined cDNA sequence for clone | 63695399 R0677: C04 |
| SEQ ID NO: 1368 | is the determined cDNA sequence for clone | 63695400 R0677: C05 |
| SEQ ID NO: 1369 | is the determined cDNA sequence for clone | 63695401 R0677: C06 |
| SEQ ID NO: 1370 | is the determined cDNA sequence for clone | 63695402 R0677: C07 |
| SEQ ID NO: 1371 | is the determined cDNA sequence for clone | 63695403 R0677: C08 |
| SEQ ID NO: 1372 | is the determined cDNA sequence for clone | 63695404 R0677: C09 |
| SEQ ID NO: 1373 | is the determined cDNA sequence for clone | 63695405 R0677: C10 |
| SEQ ID NO: 1374 | is the determined cDNA sequence for clone | 63695406 R0677: C11 |
| SEQ ID NO: 1375 | is the determined cDNA sequence for clone | 63695408 R0677: D01 |
| SEQ ID NO: 1376 | is the determined cDNA sequence for clone | 63695409 R0677: D02 |
| SEQ ID NO: 1377 | is the determined cDNA sequence for clone | 63695411 R0677: D04 |
| SEQ ID NO: 1378 | is the determined cDNA sequence for clone | 63695412 R0677: D05 |
| SEQ ID NO: 1379 | is the determined cDNA sequence for clone | 63695413 R0677: D06 |
| SEQ ID NO: 1380 | is the determined cDNA sequence for clone | 63695414 R0677: D07 |
| SEQ ID NO: 1381 | is the determined cDNA sequence for clone | 63695415 R0677: D08 |
| SEQ ID NO: 1382 | is the determined cDNA sequence for clone | 63695416 R0677: D09 |
| SEQ ID NO: 1383 | is the determined cDNA sequence for clone | 63695418 R0677: D11 |
| SEQ ID NO: 1384 | is the determined cDNA sequence for clone | 63695419 R0677: D12 |
| SEQ ID NO: 1385 | is the determined cDNA sequence for clone | 63695420 R0677: E01 |
| SEQ ID NO: 1386 | is the determined cDNA sequence for clone | 63695421 R0677: E02 |
| SEQ ID NO: 1387 | is the determined cDNA sequence for clone | 63695422 R0677: E03 |
| SEQ ID NO: 1388 | is the determined cDNA sequence for clone | 63695423 R0677: E04 |
| SEQ ID NO: 1389 | is the determined cDNA sequence for clone | 63695424 R0677: E05 |
| SEQ ID NO: 1390 | is the determined cDNA sequence for clone | 63695425 R0677: E06 |
| SEQ ID NO: 1391 | is the determined cDNA sequence for clone | 63695426 R0677: E07 |
| SEQ ID NO: 1392 | is the determined cDNA sequence for clone | 63695427 R0677: E08 |
| SEQ ID NO: 1393 | is the determined cDNA sequence for clone | 63695428 R0677: E09 |
| SEQ ID NO: 1394 | is the determined cDNA sequence for clone | 63695429 R0677: E10 |
| SEQ ID NO: 1395 | is the determined cDNA sequence for clone | 63695430 R0677: E11 |
| SEQ ID NO: 1396 | is the determined cDNA sequence for clone | 63695431 R0677: E12 |
| SEQ ID NO: 1397 | is the determined cDNA sequence for clone | 63695432 R0677: F01 |
| SEQ ID NO: 1398 | is the determined cDNA sequence for clone | 63695433 R0677: F02 |
| SEQ ID NO: 1399 | is the determined cDNA sequence for clone | 63695434 R0677: F03 |
| SEQ ID NO: 1400 | is the determined cDNA sequence for clone | 63695435 R0677: F04 |
| SEQ ID NO: 1401 | is the determined cDNA sequence for clone | 63695436 R0677: F05 |
| SEQ ID NO: 1402 | is the determined cDNA sequence for clone | 63695437 R0677: F06 |
| SEQ ID NO: 1403 | is the determined cDNA sequence for clone | 63695439 R0677: F08 |
| SEQ ID NO: 1404 | is the determined cDNA sequence for clone | 63695440 R0677: F09 |
| SEQ ID NO: 1405 | is the determined cDNA sequence for clone | 63695442 R0677: F11 |
| SEQ ID NO: 1406 | is the determined cDNA sequence for clone | 63695443 R0677: F12 |
| SEQ ID NO: 1407 | is the determined cDNA sequence for clone | 63695444 R0677: G01 |
| SEQ ID NO: 1408 | is the determined cDNA sequence for clone | 63695445 R0677: G02 |
| SEQ ID NO: 1409 | is the determined cDNA sequence for clone | 63695446 R0677: G03 |
| SEQ ID NO: 1410 | is the determined cDNA sequence for clone | 63695447 R0677: G04 |
| SEQ ID NO: 1411 | is the determined cDNA sequence for clone | 63695448 R0677: G05 |
| SEQ ID NO: 1412 | is the determined cDNA sequence for clone | 63695449 R0677: G06 |
| SEQ ID NO: 1413 | is the determined cDNA sequence for clone | 63695450 R0677: G07 |
| SEQ ID NO: 1414 | is the determined cDNA sequence for clone | 63695451 R0677: G08 |
| SEQ ID NO: 1415 | is the determined cDNA sequence for clone | 63695452 R0677: G09 |
| SEQ ID NO: 1416 | is the determined cDNA sequence for clone | 63695453 R0677: G10 |
| SEQ ID NO: 1417 | is the determined cDNA sequence for clone | 63695454 R0677: G11 |
| SEQ ID NO: 1418 | is the determined cDNA sequence for clone | 63695455 R0677: G12 |
| SEQ ID NO: 1419 | is the determined cDNA sequence for clone | 63695456 R0677: H01 |
| SEQ ID NO: 1420 | is the determined cDNA sequence for clone | 63695457 R0677: H02 |
| SEQ ID NO: 1421 | is the determined cDNA sequence for clone | 63695458 R0677: H03 |
| SEQ ID NO: 1422 | is the determined cDNA sequence for clone | 63695459 R0677: H04 |
| SEQ ID NO: 1423 | is the determined cDNA sequence for clone | 63695460 R0677: H05 |
| SEQ ID NO: 1424 | is the determined cDNA sequence for clone | 63695461 R0677: H06 |
| SEQ ID NO: 1425 | is the determined cDNA sequence for clone | 63695462 R0677: H07 |
| SEQ ID NO: 1426 | is the determined cDNA sequence for clone | 63695463 R0677: H08 |
| SEQ ID NO: 1427 | is the determined cDNA sequence for clone | 63695464 R0677: H09 |
| SEQ ID NO: 1428 | is the determined cDNA sequence for clone | 63695465 R0677: H10 |
| SEQ ID NO: 1429 | is the determined cDNA sequence for clone | 63695466 R0677: H11 |
| SEQ ID NO: 1430 | is the determined cDNA sequence for clone | 63708283 R0678: A02 |
| SEQ ID NO: 1431 | is the determined cDNA sequence for clone | 63708284 R0678: A03 |
| SEQ ID NO: 1432 | is the determined cDNA sequence for clone | 63708285 R0678: A05 |
| SEQ ID NO: 1433 | is the determined cDNA sequence for clone | 63708286 R0678: A06 |
| SEQ ID NO: 1434 | is the determined cDNA sequence for clone | 63708287 R0678: A07 |
| SEQ ID NO: 1435 | is the determined cDNA sequence for clone | 63708289 R0678: A09 |
| SEQ ID NO: 1436 | is the determined cDNA sequence for clone | 63708290 R0678: A10 |
| SEQ ID NO: 1437 | is the determined cDNA sequence for clone | 63708291 R0678: A11 |
| SEQ ID NO: 1438 | is the determined cDNA sequence for clone | 63708292 R0678: A12 |
| SEQ ID NO: 1439 | is the determined cDNA sequence for clone | 63708293 R0678: B01 |
| SEQ ID NO: 1440 | is the determined cDNA sequence for clone | 63708294 R0678: B02 |
| SEQ ID NO: 1441 | is the determined cDNA sequence for clone | 63708295 R0678: B03 |
| SEQ ID NO: 1442 | is the determined cDNA sequence for clone | 63708296 R0678: B04 |
| SEQ ID NO: 1443 | is the determined cDNA sequence for clone | 63708297 R0678: B05 |
| SEQ ID NO: 1444 | is the determined cDNA sequence for clone | 63708298 R0678: B06 |
| SEQ ID NO: 1445 | is the determined cDNA sequence for clone | 63708299 R0678: B07 |
| SEQ ID NO: 1446 | is the determined cDNA sequence for clone | 63708300 R0678: B08 |
| SEQ ID NO: 1447 | is the determined cDNA sequence for clone | 63708302 R0678: B10 |
| SEQ ID NO: 1448 | is the determined cDNA sequence for clone | 63708304 R0678: B12 |
| SEQ ID NO: 1449 | is the determined cDNA sequence for clone | 63708305 R0678: C01 |
| SEQ ID NO: 1450 | is the determined cDNA sequence for clone | 63708306 R0678: C02 |
| SEQ ID NO: 1451 | is the determined cDNA sequence for clone | 63708307 R0678: C03 |
| SEQ ID NO: 1452 | is the determined cDNA sequence for clone | 63708308 R0678: C04 |
| SEQ ID NO: 1453 | is the determined cDNA sequence for clone | 63708309 R0678: C05 |
| SEQ ID NO: 1454 | is the determined cDNA sequence for clone | 63708311 R0678: C07 |
| SEQ ID NO: 1455 | is the determined cDNA sequence for clone | 63708313 R0678: C09 |
| SEQ ID NO: 1456 | is the determined cDNA sequence for clone | 63708314 R0678: C10 |
| SEQ ID NO: 1457 | is the determined cDNA sequence for clone | 63708315 R0678: C11 |
| SEQ ID NO: 1458 | is the determined cDNA sequence for clone | 63708316 R0678: C12 |
| SEQ ID NO: 1459 | is the determined cDNA sequence for clone | 63708317 R0678: D01 |
| SEQ ID NO: 1460 | is the determined cDNA sequence for clone | 63708318 R0678: D02 |
| SEQ ID NO: 1461 | is the determined cDNA sequence for clone | 63708319 R0678: D03 |
| SEQ ID NO: 1462 | is the determined cDNA sequence for clone | 63708321 R0678: D05 |
| SEQ ID NO: 1463 | is the determined cDNA sequence for clone | 63708322 R0678: D06 |
| SEQ ID NO: 1464 | is the determined cDNA sequence for clone | 63708323 R0678: D07 |
| SEQ ID NO: 1465 | is the determined cDNA sequence for clone | 63708324 R0678: D08 |
| SEQ ID NO: 1466 | is the determined cDNA sequence for clone | 63708325 R0678: D09 |
| SEQ ID NO: 1467 | is the determined cDNA sequence for clone | 63708326 R0678: D10 |
| SEQ ID NO: 1468 | is the determined cDNA sequence for clone | 63708327 R0678: D11 |
| SEQ ID NO: 1469 | is the determined cDNA sequence for clone | 63708328 R0678: D12 |
| SEQ ID NO: 1470 | is the determined cDNA sequence for clone | 63708330 R0678: E02 |
| SEQ ID NO: 1471 | is the determined cDNA sequence for clone | 63708331 R0678: E03 |
| SEQ ID NO: 1472 | is the determined cDNA sequence for clone | 63708332 R0678: E04 |
| SEQ ID NO: 1473 | is the determined cDNA sequence for clone | 63708333 R0678: E05 |
| SEQ ID NO: 1474 | is the determined cDNA sequence for clone | 63708334 R0678: E06 |
| SEQ ID NO: 1475 | is the determined cDNA sequence for clone | 63708335 R0678: E07 |
| SEQ ID NO: 1476 | is the determined cDNA sequence for clone | 63708336 R0678: E08 |
| SEQ ID NO: 1477 | is the determined cDNA sequence for clone | 63708337 R0678: E09 |
| SEQ ID NO: 1478 | is the determined cDNA sequence for clone | 63708338 R0678: E10 |
| SEQ ID NO: 1479 | is the determined cDNA sequence for clone | 63708339 R0678: E11 |
| SEQ ID NO: 1480 | is the determined cDNA sequence for clone | 63708340 R0678: E12 |
| SEQ ID NO: 1481 | is the determined cDNA sequence for clone | 63708341 R0678: F01 |
| SEQ ID NO: 1482 | is the determined cDNA sequence for clone | 63708342 R0678: F02 |
| SEQ ID NO: 1483 | is the determined cDNA sequence for clone | 63708343 R0678: F03 |
| SEQ ID NO: 1484 | is the determined cDNA sequence for clone | 63708344 R0678: F04 |
| SEQ ID NO: 1485 | is the determined cDNA sequence for clone | 63708345 R0678: F05 |
| SEQ ID NO: 1486 | is the determined cDNA sequence for clone | 63708346 R0678: F06 |
| SEQ ID NO: 1487 | is the determined cDNA sequence for clone | 63708347 R0678: F07 |
| SEQ ID NO: 1488 | is the determined cDNA sequence for clone | 63708348 R0678: F08 |
| SEQ ID NO: 1489 | is the determined cDNA sequence for clone | 63708349 R0678: F09 |
| SEQ ID NO: 1490 | is the determined cDNA sequence for clone | 63708350 R0678: F10 |
| SEQ ID NO: 1491 | is the determined cDNA sequence for clone | 63708352 R0678: F12 |
| SEQ ID NO: 1492 | is the determined cDNA sequence for clone | 63708354 R0678: G02 |
| SEQ ID NO: 1493 | is the determined cDNA sequence for clone | 63708355 R0678: G03 |
| SEQ ID NO: 1494 | is the determined cDNA sequence for clone | 63708356 R0678: G04 |
| SEQ ID NO: 1495 | is the determined cDNA sequence for clone | 63708357 R0678: G05 |
| SEQ ID NO: 1496 | is the determined cDNA sequence for clone | 63708358 R0678: G06 |
| SEQ ID NO: 1497 | is the determined cDNA sequence for clone | 63708359 R0678: G07 |
| SEQ ID NO: 1498 | is the determined cDNA sequence for clone | 63708361 R0678: G09 |
| SEQ ID NO: 1499 | is the determined cDNA sequence for clone | 63708362 R0678: G10 |
| SEQ ID NO: 1500 | is the determined cDNA sequence for clone | 63708363 R0678: G11 |
| SEQ ID NO: 1501 | is the determined cDNA sequence for clone | 63708365 R0678: H01 |
| SEQ ID NO: 1502 | is the determined cDNA sequence for clone | 63708366 R0678: H02 |
| SEQ ID NO: 1503 | is the determined cDNA sequence for clone | 63708367 R0678: H03 |
| SEQ ID NO: 1504 | is the determined cDNA sequence for clone | 63708370 R0678: H06 |
| SEQ ID NO: 1505 | is the determined cDNA sequence for clone | 63708371 R0678: H07 |
| SEQ ID NO: 1506 | is the determined cDNA sequence for clone | 63708372 R0678: H08 |
| SEQ ID NO: 1507 | is the determined cDNA sequence for clone | 63708373 R0678: H09 |
| SEQ ID NO: 1508 | is the determined cDNA sequence for clone | 63708374 R0678: H10 |
| SEQ ID NO: 1509 | is the determined cDNA sequence for clone | 63708375 R0678: H11 |
| SEQ ID NO: 1510 | is the determined cDNA sequence for clone | 63695560 R0679: A02 |
| SEQ ID NO: 1511 | is the determined cDNA sequence for clone | 63695561 R0679: A03 |
| SEQ ID NO: 1512 | is the determined cDNA sequence for clone | 63695562 R0679: A05 |
| SEQ ID NO: 1513 | is the determined cDNA sequence for clone | 63695563 R0679: A06 |
| SEQ ID NO: 1514 | is the determined cDNA sequence for clone | 63695564 R0679: A07 |
| SEQ ID NO: 1515 | is the determined cDNA sequence for clone | 63695565 R0679: A08 |
| SEQ ID NO: 1516 | is the determined cDNA sequence for clone | 63695566 R0679: A09 |
| SEQ ID NO: 1517 | is the determined cDNA sequence for clone | 63695567 R0679: A10 |
| SEQ ID NO: 1518 | is the determined cDNA sequence for clone | 63695568 R0679: A11 |
| SEQ ID NO: 1519 | is the determined cDNA sequence for clone | 63695569 R0679: A12 |
| SEQ ID NO: 1520 | is the determined cDNA sequence for clone | 63695570 R0679: B01 |
| SEQ ID NO: 1521 | is the determined cDNA sequence for clone | 63695571 R0679: B02 |
| SEQ ID NO: 1522 | is the determined cDNA sequence for clone | 63695572 R0679: B03 |
| SEQ ID NO: 1523 | is the determined cDNA sequence for clone | 63695573 R0679: B04 |
| SEQ ID NO: 1524 | is the determined cDNA sequence for clone | 63695574 R0679: B05 |
| SEQ ID NO: 1525 | is the determined cDNA sequence for clone | 63695575 R0679: B06 |
| SEQ ID NO: 1526 | is the determined cDNA sequence for clone | 63695576 R0679: B07 |
| SEQ ID NO: 1527 | is the determined cDNA sequence for clone | 63695577 R0679: B08 |
| SEQ ID NO: 1528 | is the determined cDNA sequence for clone | 63695578 R0679: B09 |
| SEQ ID NO: 1529 | is the determined cDNA sequence for clone | 63695579 R0679: B10 |
| SEQ ID NO: 1530 | is the determined cDNA sequence for clone | 63695580 R0679: B11 |
| SEQ ID NO: 1531 | is the determined cDNA sequence for clone | 63695581 R0679: B12 |
| SEQ ID NO: 1532 | is the determined cDNA sequence for clone | 63695582 R0679: C01 |
| SEQ ID NO: 1533 | is the determined cDNA sequence for clone | 63695583 R0679: C02 |
| SEQ ID NO: 1534 | is the determined cDNA sequence for clone | 63695586 R0679: C05 |
| SEQ ID NO: 1535 | is the determined cDNA sequence for clone | 63695587 R0679: C06 |
| SEQ ID NO: 1536 | is the determined cDNA sequence for clone | 63695589 R0679: C08 |
| SEQ ID NO: 1537 | is the determined cDNA sequence for clone | 63695590 R0679: C09 |
| SEQ ID NO: 1538 | is the determined cDNA sequence for clone | 63695591 R0679: C10 |
| SEQ ID NO: 1539 | is the determined cDNA sequence for clone | 63695592 R0679: C11 |
| SEQ ID NO: 1540 | is the determined cDNA sequence for clone | 63695593 R0679: C12 |
| SEQ ID NO: 1541 | is the determined cDNA sequence for clone | 63695594 R0679: D01 |
| SEQ ID NO: 1542 | is the determined cDNA sequence for clone | 63695595 R0679: D02 |
| SEQ ID NO: 1543 | is the determined cDNA sequence for clone | 63695596 R0679: D03 |
| SEQ ID NO: 1544 | is the determined cDNA sequence for clone | 63695597 R0679: D04 |
| SEQ ID NO: 1545 | is the determined cDNA sequence for clone | 63695598 R0679: D05 |
| SEQ ID NO: 1546 | is the determined cDNA sequence for clone | 63695599 R0679: D06 |
| SEQ ID NO: 1547 | is the determined cDNA sequence for clone | 63695600 R0679: D07 |
| SEQ ID NO: 1548 | is the determined cDNA sequence for clone | 63695602 R0679: D09 |
| SEQ ID NO: 1549 | is the determined cDNA sequence for clone | 63695603 R0679: D10 |
| SEQ ID NO: 1550 | is the determined cDNA sequence for clone | 63695604 R0679: D11 |
| SEQ ID NO: 1551 | is the determined cDNA sequence for clone | 63695605 R0679: D12 |
| SEQ ID NO: 1552 | is the determined cDNA sequence for clone | 63695606 R0679: E01 |
| SEQ ID NO: 1553 | is the determined cDNA sequence for clone | 63695608 R0679: E03 |
| SEQ ID NO: 1554 | is the determined cDNA sequence for clone | 63695609 R0679: E04 |
| SEQ ID NO: 1555 | is the determined cDNA sequence for clone | 63695610 R0679: E05 |
| SEQ ID NO: 1556 | is the determined cDNA sequence for clone | 63695611 R0679: E06 |
| SEQ ID NO: 1557 | is the determined cDNA sequence for clone | 63695612 R0679: E07 |
| SEQ ID NO: 1558 | is the determined cDNA sequence for clone | 63695613 R0679: E08 |
| SEQ ID NO: 1559 | is the determined cDNA sequence for clone | 63695614 R0679: E09 |
| SEQ ID NO: 1560 | is the determined cDNA sequence for clone | 63695615 R0679: E10 |
| SEQ ID NO: 1561 | is the determined cDNA sequence for clone | 63695616 R0679: E11 |
| SEQ ID NO: 1562 | is the determined cDNA sequence for clone | 63695617 R0679: E12 |
| SEQ ID NO: 1563 | is the determined cDNA sequence for clone | 63695618 R0679: F01 |
| SEQ ID NO: 1564 | is the determined cDNA sequence for clone | 63695619 R0679: F02 |
| SEQ ID NO: 1565 | is the determined cDNA sequence for clone | 63695620 R0679: F03 |
| SEQ ID NO: 1566 | is the determined cDNA sequence for clone | 63695622 R0679: F05 |
| SEQ ID NO: 1567 | is the determined cDNA sequence for clone | 63695623 R0679: F06 |
| SEQ ID NO: 1568 | is the determined cDNA sequence for clone | 63695624 R0679: F07 |
| SEQ ID NO: 1569 | is the determined cDNA sequence for clone | 63695625 R0679: F08 |
| SEQ ID NO: 1570 | is the determined cDNA sequence for clone | 63695626 R0679: F09 |
| SEQ ID NO: 1571 | is the determined cDNA sequence for clone | 63695627 R0679: F10 |
| SEQ ID NO: 1572 | is the determined cDNA sequence for clone | 63695629 R0679: F12 |
| SEQ ID NO: 1573 | is the determined cDNA sequence for clone | 63695630 R0679: G01 |
| SEQ ID NO: 1574 | is the determined cDNA sequence for clone | 63695631 R0679: G02 |
| SEQ ID NO: 1575 | is the determined cDNA sequence for clone | 63695633 R0679: G04 |
| SEQ ID NO: 1576 | is the determined cDNA sequence for clone | 63695635 R0679: G06 |
| SEQ ID NO: 1577 | is the determined cDNA sequence for clone | 63695636 R0679: G07 |
| SEQ ID NO: 1578 | is the determined cDNA sequence for clone | 63695637 R0679: G08 |
| SEQ ID NO: 1579 | is the determined cDNA sequence for clone | 63695640 R0679: G11 |
| SEQ ID NO: 1580 | is the determined cDNA sequence for clone | 63695641 R0679: G12 |
| SEQ ID NO: 1581 | is the determined cDNA sequence for clone | 63695642 R0679: H01 |
| SEQ ID NO: 1582 | is the determined cDNA sequence for clone | 63695643 R0679: H02 |
| SEQ ID NO: 1583 | is the determined cDNA sequence for clone | 63695644 R0679: H03 |
| SEQ ID NO: 1584 | is the determined cDNA sequence for clone | 63695645 R0679: H04 |
| SEQ ID NO: 1585 | is the determined cDNA sequence for clone | 63695646 R0679: H05 |
| SEQ ID NO: 1586 | is the determined cDNA sequence for clone | 63695647 R0679: H06 |
| SEQ ID NO: 1587 | is the determined cDNA sequence for clone | 63695649 R0679: H08 |
| SEQ ID NO: 1588 | is the determined cDNA sequence for clone | 63695650 R0679: H09 |
| SEQ ID NO: 1589 | is the determined cDNA sequence for clone | 63695652 R0679: H11 |
| SEQ ID NO: 1590 | is the determined cDNA sequence for clone | 63695468 R0680: A03 |
| SEQ ID NO: 1591 | is the determined cDNA sequence for clone | 63695469 R0680: A05 |
| SEQ ID NO: 1592 | is the determined cDNA sequence for clone | 63695470 R0680: A06 |
| SEQ ID NO: 1593 | is the determined cDNA sequence for clone | 63695471 R0680: A07 |
| SEQ ID NO: 1594 | is the determined cDNA sequence for clone | 63695472 R0680: A08 |
| SEQ ID NO: 1595 | is the determined cDNA sequence for clone | 63695473 R0680: A09 |
| SEQ ID NO: 1596 | is the determined cDNA sequence for clone | 63695474 R0680: A10 |
| SEQ ID NO: 1597 | is the determined cDNA sequence for clone | 63695475 R0680: A11 |
| SEQ ID NO: 1598 | is the determined cDNA sequence for clone | 63695476 R0680: A12 |
| SEQ ID NO: 1599 | is the determined cDNA sequence for clone | 63695477 R0680: B01 |
| SEQ ID NO: 1600 | is the determined cDNA sequence for clone | 63695478 R0680: B02 |
| SEQ ID NO: 1601 | is the determined cDNA sequence for clone | 63695480 R0680: B04 |
| SEQ ID NO: 1602 | is the determined cDNA sequence for clone | 63695482 R0680: B06 |
| SEQ ID NO: 1603 | is the determined cDNA sequence for clone | 63695483 R0680: B07 |
| SEQ ID NO: 1604 | is the determined cDNA sequence for clone | 63695484 R0680: B08 |
| SEQ ID NO: 1605 | is the determined cDNA sequence for clone | 63695485 R0680: B09 |
| SEQ ID NO: 1606 | is the determined cDNA sequence for clone | 63695486 R0680: B10 |
| SEQ ID NO: 1607 | is the determined cDNA sequence for clone | 63695487 R0680: B11 |
| SEQ ID NO: 1608 | is the determined cDNA sequence for clone | 63695488 R0680: B12 |
| SEQ ID NO: 1609 | is the determined cDNA sequence for clone | 63695489 R0680: C01 |
| SEQ ID NO: 1610 | is the determined cDNA sequence for clone | 63695490 R0680: C02 |
| SEQ ID NO: 1611 | is the determined cDNA sequence for clone | 63695491 R0680: C03 |
| SEQ ID NO: 1612 | is the determined cDNA sequence for clone | 63695492 R0680: C04 |
| SEQ ID NO: 1613 | is the determined cDNA sequence for clone | 63695495 R0680: C07 |
| SEQ ID NO: 1614 | is the determined cDNA sequence for clone | 63695496 R0680: C08 |
| SEQ ID NO: 1615 | is the determined cDNA sequence for clone | 63695497 R0680: C09 |
| SEQ ID NO: 1616 | is the determined cDNA sequence for clone | 63695498 R0680: C10 |
| SEQ ID NO: 1617 | is the determined cDNA sequence for clone | 63695499 R0680: C11 |
| SEQ ID NO: 1618 | is the determined cDNA sequence for clone | 63695501 R0680: D01 |
| SEQ ID NO: 1619 | is the determined cDNA sequence for clone | 63695502 R0680: D02 |
| SEQ ID NO: 1620 | is the determined cDNA sequence for clone | 63695503 R0680: D03 |
| SEQ ID NO: 1621 | is the determined cDNA sequence for clone | 63695504 R0680: D04 |
| SEQ ID NO: 1622 | is the determined cDNA sequence for clone | 63695507 R0680: D07 |
| SEQ ID NO: 1623 | is the determined cDNA sequence for clone | 63695509 R0680: D09 |
| SEQ ID NO: 1624 | is the determined cDNA sequence for clone | 63695510 R0680: D10 |
| SEQ ID NO: 1625 | is the determined cDNA sequence for clone | 63695511 R0680: D11 |
| SEQ ID NO: 1626 | is the determined cDNA sequence for clone | 63695512 R0680: D12 |
| SEQ ID NO: 1627 | is the determined cDNA sequence for clone | 63695513 R0680: E01 |
| SEQ ID NO: 1628 | is the determined cDNA sequence for clone | 63695515 R0680: E03 |
| SEQ ID NO: 1629 | is the determined cDNA sequence for clone | 63695516 R0680: E04 |
| SEQ ID NO: 1630 | is the determined cDNA sequence for clone | 63695518 R0680: E06 |
| SEQ ID NO: 1631 | is the determined cDNA sequence for clone | 63695519 R0680: E07 |
| SEQ ID NO: 1632 | is the determined cDNA sequence for clone | 63695520 R0680: E08 |
| SEQ ID NO: 1633 | is the determined cDNA sequence for clone | 63695521 R0680: E09 |
| SEQ ID NO: 1634 | is the determined cDNA sequence for clone | 63695522 R0680: E10 |
| SEQ ID NO: 1635 | is the determined cDNA sequence for clone | 63695523 R0680: E11 |
| SEQ ID NO: 1636 | is the determined cDNA sequence for clone | 63695524 R0680: E12 |
| SEQ ID NO: 1637 | is the determined cDNA sequence for clone | 63695525 R0680: F01 |
| SEQ ID NO: 1638 | is the determined cDNA sequence for clone | 63695526 R0680: F02 |
| SEQ ID NO: 1639 | is the determined cDNA sequence for clone | 63695527 R0680: F03 |
| SEQ ID NO: 1640 | is the determined cDNA sequence for clone | 63695528 R0680: F04 |
| SEQ ID NO: 1641 | is the determined cDNA sequence for clone | 63695530 R0680: F06 |
| SEQ ID NO: 1642 | is the determined cDNA sequence for clone | 63695532 R0680: F08 |
| SEQ ID NO: 1643 | is the determined cDNA sequence for clone | 63695534 R0680: F10 |
| SEQ ID NO: 1644 | is the determined cDNA sequence for clone | 63695535 R0680: F11 |
| SEQ ID NO: 1645 | is the determined cDNA sequence for clone | 63695536 R0680: F12 |
| SEQ ID NO: 1646 | is the determined cDNA sequence for clone | 63695537 R0680: G01 |
| SEQ ID NO: 1647 | is the determined cDNA sequence for clone | 63695538 R0680: G02 |
| SEQ ID NO: 1648 | is the determined cDNA sequence for clone | 63695539 R0680: G03 |
| SEQ ID NO: 1649 | is the determined cDNA sequence for clone | 63695540 R0680: G04 |
| SEQ ID NO: 1650 | is the determined cDNA sequence for clone | 63695542 R0680: G06 |
| SEQ ID NO: 1651 | is the determined cDNA sequence for clone | 63695544 R0680: G08 |
| SEQ ID NO: 1652 | is the determined cDNA sequence for clone | 63695545 R0680: G09 |
| SEQ ID NO: 1653 | is the determined cDNA sequence for clone | 63695546 R0680: G10 |
| SEQ ID NO: 1654 | is the determined cDNA sequence for clone | 63695547 R0680: G11 |
| SEQ ID NO: 1655 | is the determined cDNA sequence for clone | 63695549 R0680: H01 |
| SEQ ID NO: 1656 | is the determined cDNA sequence for clone | 63695551 R0680: H03 |
| SEQ ID NO: 1657 | is the determined cDNA sequence for clone | 63695552 R0680: H04 |
| SEQ ID NO: 1658 | is the determined cDNA sequence for clone | 63695554 R0680: H06 |
| SEQ ID NO: 1659 | is the determined cDNA sequence for clone | 63695556 R0680: H08 |
| SEQ ID NO: 1660 | is the determined cDNA sequence for clone | 63695559 R0680: H11 |
| SEQ ID NO: 1661 | is the determined cDNA sequence for clone | 673.A9 |
| SEQ ID NO: 1662 | is the determined cDNA sequence for clone | 673.H12 |
| SEQ ID NO: 1663 | is the determined cDNA sequence for clone | 674.A7.GI: 12728304 |
| SEQ ID NO: 1664 | is the determined cDNA sequence for clone | 674.A7 |
| SEQ ID NO: 1665 | is the determined cDNA sequence for clone | 675.G9.GI: 12736649 |
| SEQ ID NO: 1666 | is the determined cDNA sequence for clone | 675.G9 |
| SEQ ID NO: 1667 | is the determined cDNA sequence for clone | 675.A11.GI: 10435821 |
| SEQ ID NO: 1668 | is the determined cDNA sequence for clone | 675.A11 |
| SEQ ID NO: 1669 | is the determined cDNA sequence for clone | 676.F9 |
| SEQ ID NO: 1670 | is the determined cDNA sequence for clone | 677.F11 |
| SEQ ID NO: 1671 | is the determined cDNA sequence for clone | 680.F1.GI: 3088574 |
| SEQ ID NO: 1672 | is the determined cDNA sequence for clone | 680.F1 |
| SEQ ID NO: 1673 | is the determined cDNA sequence for clone | 680.H3.GI: 12652924 |
| SEQ ID NO: 1674 | is the determined cDNA sequence for clone | 680.H3 |
| SEQ ID NO: 1675 | is the determined cDNA sequence for clone | 680.B11 |
| SEQ ID NO: 1676 | is the determined cDNA sequence for clone | 685.F11 |
| SEQ ID NO: 1677 | is the determined cDNA sequence for clone | 687.B3.72249 |
| SEQ ID NO: 1678 | is the determined cDNA sequence for clone | 678.D2.GI: 12734542 |
| SEQ ID NO: 1679 | is the determined cDNA sequence for clone | 678.D2.72899 |
| SEQ ID NO: 1680 | is the determined cDNA sequence for clone | 683.G3.GI: 4185790 |
| SEQ ID NO: 1681 | is the determined cDNA sequence for clone | 683.G3.70426 |
| SEQ ID NO: 1682 | is the determined cDNA sequence for clone | 673.E12.GI: 10436905 |
| SEQ ID NO: 1683 | is the determined cDNA sequence for clone | 673.E12.72901 |
| SEQ ID NO: 1684 | is the determined cDNA sequence for clone | 672.E3 |
| SEQ ID NO: 1685 | is the determined cDNA sequence for clone | 672.E3.72233 |
| SEQ ID NO: 1686 | is the determined cDNA sequence for clone | 677.C7.GI: 10434626 |
| SEQ ID NO: 1687 | is the determined cDNA sequence for clone | 677.C7.72240 |
| SEQ ID NO: 1688 | is the determined cDNA sequence for clone | 678.E10.GI: 12733361 |
| SEQ ID NO: 1689 | is the determined cDNA sequence for clone | 678.E10.72242 |
| SEQ ID NO: 1690 | is the determined cDNA sequence for clone | 679.C11.GI: 13111934 |
| SEQ ID NO: 1691 | is the determined cDNA sequence for clone | 679.C11.72243 |
| SEQ ID NO: 1692 | is the determined cDNA sequence for clone | 674.D10.71575 |
| SEQ ID NO: 1693 | is the determined cDNA sequence for clone | 664.B3.GI: 11526264 |
| SEQ ID NO: 1694 | is the determined cDNA sequence for clone | 664.B3.71569 |
| SEQ ID NO: 1695 | is the determined cDNA sequence for clone | 670.A3.71571 |
| SEQ ID NO: 1696 | is the determined cDNA sequence for clone | 665.B9.GI: 12737771. |
| SEQ ID NO: 1697 | is the determined cDNA sequence for clone | 665.B9.70580 |
| SEQ ID NO: 1698 | is the determined cDNA sequence for clone | 676G4(70581). |
| | | 678H12(70582). |
| | | 681B5(70586). |
| | | 682E4(70589) |
| SEQ ID NO: 1699 | is the determined cDNA sequence for clone | 681.F7.GI: 12737278. |
| SEQ ID NO: 1700 | is the determined cDNA sequence for clone | 681.F7.70587 |
| SEQ ID NO: 1701 | is the determined cDNA sequence for clone | 681.H11.GI: 12655152 |
| SEQ ID NO: 1702 | is the determined cDNA sequence for clone | 681.H11.70584 |
| SEQ ID NO: 1703 | is the determined cDNA sequence for clone | 681.H3.GI: 11427606 |
| SEQ ID NO: 1704 | is the determined cDNA sequence for clone | 681.H3.70588 |
| SEQ ID NO: 1705 | is the determined cDNA sequence for clone | ‘70984.1’ |
| SEQ ID NO: 1706 | is the determined cDNA sequence for clone | ‘70985.1’ |
| SEQ ID NO: 1707 | is the determined cDNA sequence for clone | ‘70990.1’ |
| SEQ ID NO: 1708 | is the determined cDNA sequence for clone | ‘70991.1’ |
| SEQ ID NO: 1709 | is the determined cDNA sequence for clone | 4.contig.GI: 11427276 |
| SEQ ID NO: 1710 | is the determined cDNA sequence for clone | ‘71023.1’ |
| SEQ ID NO: 1711 | is the determined cDNA sequence for clone | 5.contig.GI: 11422221 |
| SEQ ID NO: 1712 | is the determined cDNA sequence for clone | ‘71016.1’ |
| SEQ ID NO: 1713 | is the determined cDNA sequence for clone | ‘71003.1’ |
| SEQ ID NO: 1714 | is the determined cDNA sequence for clone | 7.contig.GI: 6330128 |
| SEQ ID NO: 1715 | is the determined cDNA sequence for clone | ‘71043.1’ |
| SEQ ID NO: 1716 | is the determined cDNA sequence for clone | 8.contig.GI: 11526264 |
| SEQ ID NO: 1717 | is the determined cDNA sequence for clone | ‘71000.1’ |
| SEQ ID NO: 1718 | is the determined cDNA sequence for clone | ‘71033.1’ |
| SEQ ID NO: 1719 | is the determined cDNA sequence for clone | 9.contig.GI: 7657545 |
| SEQ ID NO: 1720 | is the determined cDNA sequence for clone | ‘70989.1’ |
| SEQ ID NO: 1721 | is the determined cDNA sequence for clone | 10.contig.GI: 482908 |
| SEQ ID NO: 1722 | is the determined cDNA sequence for clone | ‘71040.1’ |
| SEQ ID NO: 1723 | is the determined cDNA sequence for clone | ‘71035.1’ |
| SEQ ID NO: 1724 | is the determined cDNA sequence for clone | ‘71038.1’ |
| SEQ ID NO: 1725 | is the determined cDNA sequence for clone | ‘71007.1’ |
| SEQ ID NO: 1726 | is the determined cDNA sequence for clone | ‘71047.1’ |
| SEQ ID NO: 1727 | is the determined cDNA sequence for clone | 14.contig.GI: 4096861 |
| SEQ ID NO: 1728 | is the determined cDNA sequence for clone | ‘71013.1’ |
| SEQ ID NO: 1729 | is the determined cDNA sequence for clone | ‘70983.1’ |
| SEQ ID NO: 1730 | is the determined cDNA sequence for clone | ‘71027.1’ |
| SEQ ID NO: 1731 | is the determined cDNA sequence for clone | 16.Contig.GI: 11419857 |
| SEQ ID NO: 1732 | is the determined cDNA sequence for clone | ‘71054.1’ |
| SEQ ID NO: 1733 | is the determined cDNA sequence for clone | ‘71041.1’ |
| SEQ ID NO: 1734 | is the determined cDNA sequence for clone | ‘71031.1’ |
| SEQ ID NO: 1735 | is the determined cDNA sequence for clone | ‘71034.1’ |
| SEQ ID NO: 1736 | is the determined cDNA sequence for clone | ‘71019.1’ |
| SEQ ID NO: 1737 | is the determined cDNA sequence for clone | ‘71050.1’ |
| SEQ ID NO: 1738 | is the determined cDNA sequence for clone | 23.contig.GI: 4502778 |
| SEQ ID NO: 1739 | is the determined cDNA sequence for clone | ‘71010.1’ |
| SEQ ID NO: 1740 | is the determined cDNA sequence for clone | 24.Contig.GI: 6005991 |
| SEQ ID NO: 1741 | is the determined cDNA sequence for clone | ‘71044.1’ |
| SEQ ID NO: 1742 | is the determined cDNA sequence for clone | ‘70996.1’ |
| SEQ ID NO: 1743 | is the determined cDNA sequence for clone | 26.Contig.GI: 177801 |
| SEQ ID NO: 1744 | is the determined cDNA sequence for clone | ‘71060.1’ |
| SEQ ID NO: 1745 | is the determined cDNA sequence for clone | 27.Contig.GI: 10439726 |
| SEQ ID NO: 1746 | is the determined cDNA sequence for clone | ‘71057.1’ |
| SEQ ID NO: 1747 | is the determined cDNA sequence for clone | ‘71001.1’ |
| SEQ ID NO: 1748 | is the determined cDNA sequence for clone | 29.contig.gbID.11429588 |
| SEQ ID NO: 1749 | is the determined cDNA sequence for clone | ‘70971.1’ |
| SEQ ID NO: 1750 | is the determined cDNA sequence for clone | ‘70973.1’ |
| SEQ ID NO: 1751 | is the determined cDNA sequence for clone | ‘70974.1’ |
| SEQ ID NO: 1752 | is the determined cDNA sequence for clone | ‘70975.1’ |
| SEQ ID NO: 1753 | is the determined cDNA sequence for clone | ‘70977.1’ |
| SEQ ID NO: 1754 | is the determined cDNA sequence for clone | ‘70980.1’ |
| SEQ ID NO: 1755 | is the determined cDNA sequence for clone | ‘70981.1’ |
| SEQ ID NO: 1756 | is the determined cDNA sequence for clone | ‘70982.1’ |
| SEQ ID NO: 1757 | is the determined cDNA sequence for clone | ‘70986.1’ |
| SEQ ID NO: 1758 | is the determined cDNA sequence for clone | ‘70987.1’ |
| SEQ ID NO: 1759 | is the determined cDNA sequence for clone | ‘70988.1’ |
| SEQ ID NO: 1760 | is the determined cDNA sequence for clone | ‘70997.1’ |
| SEQ ID NO: 1761 | is the determined cDNA sequence for clone | ‘70998.1’ |
| SEQ ID NO: 1762 | is the determined cDNA sequence for clone | ‘70999.1’ |
| SEQ ID NO: 1763 | is the determined cDNA sequence for clone | ‘71006.1’ |
| SEQ ID NO: 1764 | is the determined cDNA sequence for clone | ‘71008.1’ |
| SEQ ID NO: 1765 | is the determined cDNA sequence for clone | ‘71009.1’ |
| SEQ ID NO: 1766 | is the determined cDNA sequence for clone | ‘71011.1’ |
| SEQ ID NO: 1767 | is the determined cDNA sequence for clone | ‘71012.1’ |
| SEQ ID NO: 1768 | is the determined cDNA sequence for clone | ‘71018.1’ |
| SEQ ID NO: 1769 | is the determined cDNA sequence for clone | ‘71021.1’ |
| SEQ ID NO: 1770 | is the determined cDNA sequence for clone | ‘71022.1’ |
| SEQ ID NO: 1771 | is the determined cDNA sequence for clone | ‘71024.1’ |
| SEQ ID NO: 1772 | is the determined cDNA sequence for clone | ‘71028.1’ |
| SEQ ID NO: 1773 | is the determined cDNA sequence for clone | ‘71029.1’ |
| SEQ ID NO: 1774 | is the determined cDNA sequence for clone | ‘71032.1’ |
| SEQ ID NO: 1775 | is the determined cDNA sequence for clone | ‘71036.1’ |
| SEQ ID NO: 1776 | is the determined cDNA sequence for clone | ‘71037.1’ |
| SEQ ID NO: 1777 | is the determined cDNA sequence for clone | ‘71039.1’ |
| SEQ ID NO: 1778 | is the determined cDNA sequence for clone | ‘71045.1’ |
| SEQ ID NO: 1779 | is the determined cDNA sequence for clone | ‘71049.1’ |
| SEQ ID NO: 1780 | is the determined cDNA sequence for clone | ‘71051.1’ |
| SEQ ID NO: 1781 | is the determined cDNA sequence for clone | ‘71055.1’ |
| SEQ ID NO: 1782 | is the determined cDNA sequence for clone | ‘71058.1’ |
| SEQ ID NO: 1783 | is the determined cDNA sequence for clone | ‘71059.1’ |
| SEQ ID NO: 1784 | is the determined cDNA sequence for clone | ‘71062.1’ |
| SEQ ID NO: 1785 | is the determined cDNA sequence for clone | ‘71063.1’ |
| SEQ ID NO: 1786 | is the determined cDNA sequence for clone | ‘71065.1’ |
| SEQ ID NO: 1787 | is the determined cDNA sequence for clone | ‘71066.1’ |
| SEQ ID NO: 1788 | is the determined cDNA sequence for clone | 602287 Human E1A |
| | | enhancer binding protein |
| | | (EIA-F) |
| SEQ ID NO: 1789 | is the predicted amino acid sequence for | SEQ ID NO: 1788, |
| | | Human E1A enhancer |
| | | binding protein (EIA-F) |

### DETAILED DESCRIPTION OF THE INVENTION

The present invention is directed generally to compositions and their use in the therapy and diagnosis of cancer, particularly colon cancer. As described further below, illustrative compositions of the present invention include, but are not restricted to, polypeptides, particularly immunogenic polypeptides, polynucleotides encoding such polypeptides, antibodies and other binding agents, antigen presenting cells (APCs) and immune system cells (e.g., T cells).

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Polypeptide Compositions

As used herein, the term “polypeptide” ” is used in its conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

Particularly illustrative polypeptides of the present invention comprise those encoded by a polynucleotide sequence set forth in any one of SEQ ID NO:1-1788, or a sequence that hybridizes under moderately stringent conditions, or, alternatively, under highly stringent conditions, to a polynucleotide sequence set forth in any one of SEQ ID NO:1-1788. Certain other illustrative polypeptides of the invention comprise amino acid sequences as set forth in any one of SEQ ID NO:1789.

The polypeptides of the present invention are sometimes herein referred to as colon tumor proteins or colon tumor polypeptides, as an indication that their identification has been based at least in part upon their increased levels of expression in colon tumor samples. Thus, a “colon tumor polypeptide” or “colon tumor protein,” refers generally to a polypeptide sequence of the present invention, or a polynucleotide sequence encoding such a polypeptide, that is expressed in a substantial proportion of colon tumor samples, for example preferably greater than about 20%, more preferably greater than about 30%, and most preferably greater than about 50% or more of colon tumor samples tested, at a level that is at least two fold, and preferably at least five fold, greater than the level of expression in normal tissues, as determined using a representative assay provided herein. A colon tumor polypeptide sequence of the invention, based upon its increased level of expression in tumor cells, has particular utility both as a diagnostic marker as well as a therapeutic target, as further described below.

In certain preferred embodiments, the polypeptides of the invention are immunogenic, i.e., they react detectably within an immunoassay (such as an ELISA or T-cell stimulation assay) with antisera and/or T-cells from a patient with colon cancer. Screening for immunogenic activity can be performed using techniques well known to the skilled artisan. For example, such screens can be performed using methods such as those described in Harlow and Lane, Antibodies. A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides disclosed herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

In another embodiment of the invention, polypeptides are provided that comprise one or more polypeptides that are capable of eliciting T cells and/or antibodies that are immunologically reactive with one or more polypeptides described herein, or one or more polypeptides encoded by contiguous nucleic acid sequences contained in the polynucleotide sequences disclosed herein, or immunogenic fragments or variants thereof, or to one or more nucleic acid sequences which hybridize to one or more of these sequences under conditions of moderate to high stringency.

The present invention, in another aspect, provides polypeptide fragments comprising at least about 5, 10, 15, 20, 25, 50, or 100 contiguous amino acids, or more, including all intermediate lengths, of a polypeptide compositions set forth herein, such as those set forth in SEQ ID NO:1789, or those encoded by a polynucleotide sequence set forth in a sequence of SEQ ID NO:1-1788.

In another aspect, the present invention provides variants of the polypeptide compositions described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

In one preferred embodiment, the polypeptide fragments and variants provided by the present invention are immunologically reactive with an antibody and/or T-cell that reacts with a full-length polypeptide specifically set forth herein.

In another preferred embodiment, the polypeptide fragments and variants provided by the present invention exhibit a level of immunogenic activity of at least about 50%, preferably at least about 70%, and most preferably at least about 90% or more of that exhibited by a full-length polypeptide sequence specifically set forth herein.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their immunogenic activity as described herein and/or using any of a number of techniques well known in the art.

For example, certain illustrative variants of the polypeptides of the invention include those in which one or more portions, such as an N-terminal leader sequence or transmembrane domain, have been removed. Other illustrative variants include variants in which a small portion (e.g., 1-30 amino acids, preferably 5-15 amino acids) has been removed from the N- and/or C-terminal of the mature protein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5: 151-153; Myers, E. W. and Muller W. (1988) CABIOS 4: 11-17; Robinson, E. D. (1971) Comb. Theor 11: 105; Saitou, N. Nei, M. (1987) Mol. Biol. Evol. 4: 406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80: 726-730.

Alternatively, optimal alignment of sequences for comparison may be conducted by the local identity algorithm of Smith and Waterman (1981) Add. APL. Math 2: 482, by the identity alignment algorithm of Needleman and Wunsch (1970) J. Mol. Biol. 48: 443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25: 3389-3402 and Altschul et al. (1990) J. Mol. Biol. 215: 403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide may be a xenogeneic polypeptide that comprises an polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Accordingly, the present invention provides methods for purifying the xenogeneic form of the tumor proteins set forth herein, such as the polypeptide set forth in SEQ ID NO:1789, or those encoded by polynucleotide sequences set forth in SEQ ID NO:1-1788.

Therefore, one aspect of the present invention provides xenogeneic variants of the polypeptide compositions described herein. Such xenogeneic variants generally encompassed by the present invention will typically exhibit at least about 70%, 75%, 80%, 85%, 90%, 91%, 92%, 93%, 94%, 95%, 96%, 97%, 98%, or 99% or more identity along their lengths, to a polypeptide sequences set forth herein.

More particularly, the invention is directed to mouse, rat, monkey, porcine and other non-human polypeptides which can be used as xenogeneic forms of human polypeptides set forth herein, to induce immune responses directed against tumor polypeptides of the invention.

Within other illustrative embodiments, a polypeptide may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40: 39-46, 1985; Murphy et al., Proc. Natl. Acad. Sci. USA 83: 8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide as described herein together with an unrelated immunogenic protein, such as an immunogenic protein capable of eliciting a recall response. Examples of such proteins include tetanus, tuberculosis and hepatitis proteins (see, for example, Stoute et al. New Engl. J. Med., 336: 86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. Patent Application 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. Patent Application 60/158,585; see also, Skeiky et al, Infection and Immun. (1999) 67: 3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein-known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43: 265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10: 795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

Polypeptides of the invention are prepared using any of a variety of well known synthetic and/or recombinant techniques, the latter of which are further described below. Polypeptides, portions and other variants generally less than about 150 amino acids can be generated by synthetic means, using techniques well known to those of ordinary skill in the art. In one illustrative example, such polypeptides are synthesized using any of the commercially available solid-phase techniques, such as the Merrifield solid-phase synthesis method, where amino acids are sequentially added to a growing amino acid chain. See Merrifield, J. Am. Chem. Soc. 85: 2149-2146, 1963. Equipment for automated synthesis of polypeptides is commercially available from suppliers such as Perkin Elmer/Applied BioSystems Division (Foster City, Calif.), and may be operated according to the manufacturer's instructions.

In general, polypeptide compositions (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Polynucleotide Compositions

The present invention, in other aspects, provides polynucleotide compositions. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

As will be understood by those skilled in the art, the polynucleotide compositions of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be also recognized by the skilled artisan, polynucleotides of the invention may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a polypeptide/protein of the invention or a portion thereof) or may comprise a sequence that encodes a variant or derivative, preferably and immunogenic variant or derivative, of such a sequence.

Therefore, according to another aspect of the present invention, polynucleotide compositions are provided that comprise some or all of a polynucleotide sequence set forth in any one of SEQ ID NO:1-1788, complements of a polynucleotide sequence set forth in any one of SEQ ID NO:1-1788, and degenerate variants of a polynucleotide sequence set forth in any one of SEQ ID NO:1-1788. In certain preferred embodiments, the polynucleotide sequences set forth herein encode immunogenic polypeptides, as described above.

In other related embodiments, the present invention provides polynucleotide variants having substantial identity to the sequences disclosed herein in SEQ ID NO:1-1788, for example those comprising at least 70% sequence identity, preferably at least 75%, 80%, 85%, 90%, 95%, 96%, 97%, 98%, or 99% or higher, sequence identity compared to a polynucleotide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompasses homologous genes of xenogenic origin.

In additional embodiments, the present invention provides polynucleotide fragments comprising or consisting of various lengths of contiguous stretches of sequence identical to or complementary to one or more of the sequences disclosed herein. For example, polynucleotides are provided by this invention that comprise or consist of at least about 10, 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of the disclosed sequence or at both ends of the disclosed sequence.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C. or 65-70° C.

In certain preferred embodiments, the polynucleotides described above, e.g., polynucleotide variants, fragments and hybridizing sequences, encode polypeptides that are immunologically cross-reactive with a polypeptide sequence specifically set forth herein. In other preferred embodiments, such polynucleotides encode polypeptides that have a level of immunogenic activity of at least about 50%, preferably at least about 70%, and more preferably at least about 90% of that for a polypeptide sequence specifically set forth herein.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5: 151-153; Myers, E. W. and Muller W. (1988) CABIOS 4: 11-17; Robinson, E. D. (1971) Comb. Theor 11: 105; Santou, N. Nes, M. (1987) Mol. Biol. Evol. 4: 406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80: 726-730.

Alternatively, optimal alignment of sequences for comparison may be conducted by the local identity algorithm of Smith and Waterman (1981) Add. APL. Math 2: 482, by the identity alignment algorithm of Needleman and Wunsch (1970) J. Mol. Biol. 48: 443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25: 3389-3402 and Altschul et al. (1990) J. Mol. Biol. 215: 403-410, respectively. BLAST and BLAST 2.0 can be used, for example with the parameters described herein, to determine percent sequence identity for the polynucleotides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89: 10915) alignments, (B) of 50, expectation (E) of 10, M=5, N-4 and a comparison of both strands.

Preferably, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment of the invention, a mutagenesis approach, such as site-specific mutagenesis, is employed for the preparation of immunogenic variants and/or derivatives of the polypeptides described herein. By this approach, specific modifications in a polypeptide sequence can be made through mutagenesis of the underlying polynucleotides that encode them. These techniques provides a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the immunogenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

In another approach for the production of polypeptide variants of the present invention, recursive sequence recombination, as described in U.S. Pat. No. 5,837,458, may be employed. In this approach, iterative cycles of recombination and screening or selection are performed to “evolve” individual polynucleotide variants of the invention having, for example, enhanced immunogenic activity.

In other embodiments of the present invention, the polynucleotide sequences provided herein can be advantageously used as probes or primers for nucleic acid hybridization. As such, it is contemplated that nucleic acid segments that comprise or consist of a sequence region of at least about a 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequences set forth herein, or to any continuous portion of the sequences, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

According to another embodiment of the present invention, polynucleotide compositions comprising antisense oligonucleotides are provided. Antisense oligonucleotides have been demonstrated to be effective and targeted inhibitors of protein synthesis, and, consequently, provide a therapeutic approach by which a disease can be treated by inhibiting the synthesis of proteins that contribute to the disease. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. No. 5,739,119 and U.S. Pat. No. 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. 1988 Jun. 10; 240(4858): 1544-6; Vasanthakumar and Ahmed, Cancer Commun. 1989; 1(4): 225-32; Peris et al., Brain Res Mol Brain Res. 1998 Jun. 15; 57(2): 310-20; U.S. Pat. No. 5,801,154; U.S. Pat. No. 5,789,573; U.S. Pat. No. 5,718,709 and U.S. Pat. No. 5,610,288). Antisense constructs have also been described that inhibit and can be used to treat a variety of abnormal cellular proliferations, e.g. cancer (U.S. Pat. No. 5,747,470; U.S. Pat. No. 5,591,317 and U.S. Pat. No. 5,783,683).

Therefore, in certain embodiments, the present invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof. In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein. Selection of antisense compositions specific for a given gene sequence is based upon analysis of the chosen target sequence and determination of secondary structure, Tₘ, binding energy, and relative stability. Antisense compositions may be selected based upon their relative inability to form dimers, hairpins, or other secondary structures that would reduce or prohibit specific binding to the target mRNA in a host cell. Highly preferred target regions of the mRNA, are those which are at or near the AUG translation initiation codon, and those sequences which are substantially complementary to 5′ regions of the mRNA. These secondary structure analyses and target site selection considerations can be performed, for example, using v.4 of the OLIGO primer analysis software and/or the BLASTN 2.0.5 algorithm software (Altschul et al., Nucleic Acids Res. 1997, 25(17): 3389-402).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., Nucleic Acids Res. 1997 Jul. 15; 25(14): 2730-6). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane.

According to another embodiment of the invention, the polynucleotide compositions described herein are used in the design and preparation of ribozyme molecules for inhibiting expression of the tumor polypeptides and proteins of the present invention in tumor cells. Ribozymes are RNA-protein complexes that cleave nucleic acids in a site-specific fashion. Ribozymes have specific catalytic domains that possess endonuclease activity (Kim and Cech, Proc Natl Acad Sci USA. 1987 December; 84(24): 8788-92; Forster and Symons, Cell. 1987 Apr. 24; 49(2): 211-20). For example, a large number of ribozymes accelerate phosphoester transfer reactions with a high degree of specificity, often cleaving only one of several phosphoesters in an oligonucleotide substrate (Cech et al, Cell. 1981 December; 27(3 Pt 2): 487-96; Michel and Westhof, J Mol. Biol. 1990 Dec. 5; 216(3): 585-610; Reinhold-Hurek and Shub, Nature. 1992 May 14; 357(6374): 173-6). This specificity has been attributed to the requirement that the substrate bind via specific base-pairing interactions to the internal guide sequence (“IGS”) of the ribozyme prior to chemical reaction.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., Proc Natl Acad Sci USA. 1992 Aug. 15; 89(16): 7305-9). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. Nucleic Acids Res. 1992 Sep. 11; 20(17): 4559-65. Examples of hairpin motifs are described by Hampel et al. (Eur. Pat. Appl. Publ. No. EP 0360257), Hampel and Tritz, Biochemistry 1989 Jun. 13; 28(12): 4929-33; Hampel et al., Nucleic Acids Res. 1990 Jan. 25; 18(2): 299-304 and U.S. Pat. No. 5,631,359. An example of the hepatitis δ virus motif is described by Perrotta and Been, Biochemistry. 1992 Dec. 1; 31(47): 11843-52; an example of the RNaseP motif is described by Guerrier-Takada et al., Cell. 1983 December; 35(3 Pt 2): 849-57; Neurospora VS RNA ribozyme motif is described by Collins (Saville and Collins, Cell. 1990 May 18; 61(4): 685-96; Saville and Collins, Proc Natl Acad Sci USA. 1991 Oct. 1; 88(19): 8826-30; Collins and Olive, Biochemistry. 1993 Mar. 23; 32(11): 2795-9); and an example of the Group I intron is described in (U.S. Pat. No. 4,987,071). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

Ribozymes may be designed as described in Int. Pat. Appl. Publ. No. WO 93/23569 and Int. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells Ribozymes expressed from such promoters have been shown to function in mammalian cells. Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

In another embodiment of the invention, peptide nucleic acids (PNAs) compositions are provided. PNA is a DNA mimic in which the nucleobases are attached to a pseudopeptide backbone (Good and Nielsen, Antisense Nucleic Acid Drug Dev. 1997 7(4) 431-37). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol 1997 June; 15(6): 224-9). As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., Science 1991 Dec. 6; 254(5037): 1497-500; Hanvey et al., Science. 1992 Nov. 27; 258(5087): 1481-5; Hyrup and Nielsen, Bioorg Med Chem. 1996 January; 4(1): 5-23). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc or Fmoc protocols for solid-phase peptide synthesis, although other methods, including a modified Merrifield method, have been used.

PNA monomers or ready-made oligomers are commercially available from PerSeptive Biosystems (Framingham, Mass.). PNA syntheses by either Boc or Fmoc protocols are straightforward using manual or automated protocols (Norton et al., Bioorg Med Chem. 1995 April; 3(4): 437-45). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography, providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (for example, Norton et al., Bioorg Med Chem. 1995 April; 3(4): 437-45; Petersen et al., J Pept Sci. 1995 May-June; 1(3): 175-83; Orum et al., Biotechniques. 1995 September; 19(3): 472-80; Footer et al., Biochemistry. 1996 Aug. 20; 35(33): 10673-9; Griffith et al., Nucleic Acids Res. 1995 Aug. 11; 23(15): 3003-8; Pardridge et al., Proc Natl Acad Sci USA. 1995 Jun. 6; 92(12): 5592-6; Boffa et al., Proc Natl Acad Sci USA. 1995 Mar. 14; 92(6): 1901-5; Gambacorti-Passerini et al., Blood. 1996 Aug. 15; 88(4): 1411-7; Armitage et al., Proc Natl Acad Sci USA. 1997 Nov. 11; 94(23): 12320-5; Seeger et al., Biotechniques. 1997 September; 23(3): 512-7). U.S. Pat. No. 5,700,922 discusses PNA-DNA-PNA chimeric molecules and their uses in diagnostics, modulating protein in organisms, and treatment of conditions susceptible to therapeutics.

Methods of characterizing the antisense binding properties of PNAs are discussed in Rose (Anal Chem. 1993 Dec. 15; 65(24): 3545-9) and Jensen et al. (Biochemistry. 1997 Apr. 22; 36(16): 5072-7). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs that have been described and will be apparent to the skilled artisan include use in DNA strand invasion, antisense inhibition, mutational analysis, enhancers of transcription, nucleic acid purification, isolation of transcriptionally active genes, blocking of transcription factor binding, genome cleavage, biosensors, in situ hybridization, and the like.

Polynucleotide Identification Characterization and Expression

Polynucleotides compositions of the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93: 10614-10619, 1996 and Heller et al., Proc. Natl. Acad. Sci. USA 94: 2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16: 8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1: 111-19, 1991) and walking PCR (Parker et al., Nucl. Acids. Res. 19: 3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269: 202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as pBLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264: 5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153: 516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6: 307-311. Alternatively, plant promoters such as the small subunit of RUBISCO or heat shock promoters may be used (Coruzzi, G. et al. (1984) EMBO J. 3: 1671-1680; Broglie, R. et al. (1984) Science 224: 838-843; and Winter, J. et al. (1991) Results Probl. Cell Differ. 17: 85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91: 3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81: 3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20: 125-162).

In addition, a host cell strain may be chosen for its ability to modulate the expression of the inserted sequences or to process the expressed protein in the desired fashion. Such modifications of the polypeptide include, but are not limited to, acetylation, carboxylation. glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11: 223-32) and adenine phosphoribosyltransferase (Lowy, I. et al. (1990) Cell 22: 817-23) genes which can be employed in tk.sup.− or aprt.sup.− cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77: 3567-70); npt, which confers resistance to the aminoglycosides, neomycin and G-418 (Colbere-Garapin, F. et al (1981) J. Mol. Biol. 150: 1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85: 8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55: 121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158: 1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3: 263-281) while the enterokinase cleavage site provides a means for purifying the desired polypeptide from the fusion protein. A discussion of vectors which contain fusion proteins is provided in Kroll, D. J. et al. (1993; DNA Cell Biol. 12: 441-453).

In addition to recombinant production methods, polypeptides of the invention, and fragments thereof, may be produced by direct peptide synthesis using solid-phase techniques (Merrifield J. (1963) J. Am. Chem. Soc. 85: 2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Antibody Compositions Fragments Thereof and Other Binding Agents

According to another aspect, the present invention further provides binding agents, such as antibodies and antigen-binding fragments thereof, that exhibit immunological binding to a tumor polypeptide disclosed herein, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59: 439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Binding agents may be further capable of differentiating between patients with and without a cancer, such as colon cancer, using the representative assays provided herein. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6: 511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69: 2659-2662; Hochman et al. (1976) Biochem 15: 2706-2710; and Ehrlich et al. (1980) Biochem 19: 4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16): 5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349: 293-299; Lobuglio et al. (1989) Proc. Nat. Acad. Sci. USA 86: 4220-4224; Shaw et al. (1987) J. Immunol. 138: 4534-4538; and Brown et al. (1987) Cancer Res. 47: 3577-3583), rodent CDRs grafted into a human supporting FR prior to fusion with an appropriate human antibody constant domain (Riechmann et al. (1988) Nature 332: 323-327; Verhoeyen et al. (1988) Science 239: 1534-1536; and Jones et al. (1986) Nature 321: 522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59: 439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶R, ¹⁸⁸R, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

T Cell Compositions

The present invention, in another aspect, provides T cells specific for a tumor polypeptide disclosed herein, or for a variant or derivative thereof. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. No. 5,240,856; U.S. Pat. No. 5,215,926; WO 89/06280; WO 91/16116 and WO 92/07243). Alternatively, T cells may be derived from related or unrelated humans, non-human mammals, cell lines or cultures.

T cells may be stimulated with a polypeptide, polynucleotide encoding a polypeptide and/or an antigen presenting cell (APC) that expresses such a polypeptide. Such stimulation is performed under conditions and for a time sufficient to permit the generation of T cells that are specific for the polypeptide of interest. Preferably, a tumor polypeptide or polynucleotide of the invention is present within a delivery vehicle, such as a microsphere, to facilitate the generation of specific T cells.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54: 1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a tumor polypeptide, polynucleotide or APC can be expanded in number either in vitro or in vivo. Proliferation of such T cells in vitro may be accomplished in a variety of ways. For example, the T cells can be re-exposed to a tumor polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

T Cell Receptor Compositions

The T cell receptor (TCR) consists of 2 different, highly variable polypeptide chains, termed the T-cell receptor α and β chains, that are linked by a disulfide bond (Janeway, Travers, Walport. Immunobiology. Fourth Ed., 148-159. Elsevier Science Ltd/Garland Publishing. 1999). The α/β heterodimer complexes with the invariant CD3 chains at the cell membrane. This complex recognizes specific antigenic peptides bound to MHC molecules. The enormous diversity of TCR specificities is generated much like immunoglobulin diversity, through somatic gene rearrangement. The β chain genes contain over 50 variable (V), 2 diversity (D), over 10 joining (J) segments, and 2 constant region segments (C). The α chain genes contain over 70 V segments, and over 60 J segments but no D segments, as well as one C segment. During T cell development in the thymus, the D to J gene rearrangement of the β chain occurs, followed by the V gene segment rearrangement to the DJ. This functional VDJ_{β} exon is transcribed and spliced to join to a C_{β}. For the α chain, a V_{α} gene segment rearranges to a J_{α} gene segment to create the functional exon that is then transcribed and spliced to the C_{α}. Diversity is further increased during the recombination process by the random addition of P and N-nucleotides between the V, D, and J segments of the β chain and between the V and J segments in the α chain (Janeway, Travers, Walport. Immunobiology. Fourth Ed., 98 and 150. Elsevier Science Ltd/Garland Publishing. 1999).

The present invention, in another aspect, provides TCRs specific for a polypeptide disclosed herein, or for a variant or derivative thereof. In accordance with the present invention, polynucleotide and amino acid sequences are provided for the V-J or V-D-J junctional regions or parts thereof for the alpha and beta chains of the T-cell receptor which recognize tumor polypeptides described herein. In general, this aspect of the invention relates to T-cell receptors which recognize or bind tumor polypeptides presented in the context of MHC. In a preferred embodiment the tumor antigens recognized by the T-cell receptors comprise a polypeptide of the present invention. For example, cDNA encoding a TCR specific for a colon tumor peptide can be isolated from T cells specific for a tumor polypeptide using standard molecular biological and recombinant DNA techniques.

This invention further includes the T-cell receptors or analogs thereof having substantially the same function or activity as the T-cell receptors of this invention which recognize or bind tumor polypeptides. Such receptors include, but are not limited to, a fragment of the receptor, or a substitution, addition or deletion mutant of a T-cell receptor provided herein. This invention also encompasses polypeptides or peptides that are substantially homologous to the T-cell receptors provided herein or that retain substantially the same activity. The term “analog” includes any protein or polypeptide having an amino acid residue sequence substantially identical to the T-cell receptors provided herein in which one or more residues, preferably no more than 5 residues, more preferably no more than 25 residues have been conservatively substituted with a functionally similar residue and which displays the functional aspects of the T-cell receptor as described herein.

The present invention further provides for suitable mammalian host cells, for example, non-specific T cells, that are transfected with a polynucleotide encoding TCRs specific for a polypeptide described herein, thereby rendering the host cell specific for the polypeptide. The α and β chains of the TCR may be contained on separate expression vectors or alternatively, on a single expression vector that also contains an internal ribosome entry site (IRES) for cap-independent translation of the gene downstream of the IRES. Said host cells expressing TCRs specific for the polypeptide may be used, for example, for adoptive immunotherapy of colon cancer as discussed further below.

In further aspects of the present invention, cloned TCRs specific for a polypeptide recited herein may be used in a kit for the diagnosis of colon cancer. For example, the nucleic acid sequence or portions thereof, of tumor-specific TCRs can be used as probes or primers for the detection of expression of the rearranged genes encoding the specific TCR in a biological sample. Therefore, the present invention further provides for an assay for detecting messenger RNA or DNA encoding the TCR specific for a polypeptide.

Pharmaceutical Compositions

In additional embodiments, the present invention concerns formulation of one or more of the polynucleotide, polypeptide, T-cell, TCR, and/or antibody compositions disclosed herein in pharmaceutically-acceptable carriers for administration to a cell or an animal, either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Therefore, in another aspect of the present invention, pharmaceutical compositions are provided comprising one or more of the polynucleotide, polypeptide, antibody, TCR, and/or T-cell compositions described herein in combination with a physiologically acceptable carrier. In certain preferred embodiments, the pharmaceutical compositions of the invention comprise immunogenic polynucleotide and/or polypeptide compositions of the invention for use in prophylactic and theraputic vaccine applications. Vaccine preparation is generally described in, for example, M. F. Powell and M. J. Newman, eds., “Vaccine Design (the subunit and adjuvant approach),” Plenum Press (NY, 1995). Generally, such compositions will comprise one or more polynucleotide and/or polypeptide compositions of the present invention in combination with one or more immunostimulants.

It will be apparent that any of the pharmaceutical compositions described herein can contain pharmaceutically acceptable salts of the polynucleotides and polypeptides of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

In another embodiment, illustrative immunogenic compositions, e.g., vaccine compositions, of the present invention comprise DNA encoding one or more of the polypeptides as described above, such that the polypeptide is generated in situ. As noted above, the polynucleotide may be administered within any of a variety of delivery systems known to those of ordinary skill in the art. Indeed, numerous gene delivery techniques are well known in the art, such as those described by Rolland, Crit. Rev. Therap. Drug Carrier Systems 15: 143-198, 1998, and references cited therein. Appropriate polynucleotide expression systems will, of course, contain the necessary regulatory DNA regulatory sequences for expression in a patient (such as a suitable promoter and terminating signal). Alternatively, bacterial delivery systems may involve the administration of a bacterium (such as Bacillis-Calmette-Guerrin) that expresses an immunogenic portion of the polypeptide on its cell surface or secretes such an epitope.

Therefore, in certain embodiments, polynucleotides encoding immunogenic polypeptides described herein are introduced into suitable mammalian host cells for expression using any of a number of known viral-based systems. In one illustrative embodiment, retroviruses provide a convenient and effective platform for gene delivery systems. A selected nucleotide sequence encoding a polypeptide of the present invention can be inserted into a vector and packaged in retroviral particles using techniques known in the art. The recombinant virus can then be isolated and delivered to a subject. A number of illustrative retroviral systems have been described (e.g., U.S. Pat. No. 5,219,740; Miller and Rosman (1989) BioTechniques 7: 980-990; Miller, A. D. (1990) Human Gene Therapy 1: 5-14; Scarpa et al. (1991) Virology 180: 849-852; Burns et al. (1993) Proc. Natl. Acad. Sci. USA 90: 8033-8037; and Boris-Lawrie and Temin (1993) Cur. Opin. Genet. Develop. 3: 102-109.

In addition, a number of illustrative adenovirus-based systems have also been described. Unlike retroviruses which integrate into the host genome, adenoviruses persist extrachromosomally thus minimizing the risks associated with insertional mutagenesis (Haj-Ahmad and Graham (1986) J. Virol. 57: 267-274; Bett et al. (1993) J. Virol. 67: 5911-5921; Mittereder et al. (1994) Human Gene Therapy 5: 717-729; Seth et al. (1994) J. Virol. 68: 933-940; Barr et al. (1994) Gene Therapy 1: 51-58; Berkner, K. L. (1988) BioTechniques 6: 616-629; and Rich et al. (1993) Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8: 3988-3996; Vincent et al. (1990) Vaccines 90 (Cold Spring Harbor Laboratory Press); Carter, B. J. (1992) Current Opinion in Biotechnology 3: 533-539; Muzyczka, N. (1992) Current Topics in Microbiol. and Immunol. 158: 97-129; Kotin, R. M. (1994) Human Gene Therapy 5: 793-801; Shelling and Smith (1994) Gene Therapy 1: 165-169; and Zhou et al. (1994) J. Exp. Med. 179: 1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccinia virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87: 6743-6747; Fuerst et al. Proc. Natl. Acad. Sci. USA (1986) 83: 8122-8126.

Alternatively, avipoxviruses, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268: 6866-6869 and Wagner et al. Proc. Natl. Acad. Sci. USA (1992) 89: 6099-6103, can also be used for gene delivery under the invention.

Additional illustrative information on these and other known viral-based delivery systems can be found, for example, in Fisher-Hoch et al., Proc. Natl. Acad. Sci. USA 86: 317-321, 1989; Flexner et al., Ann. N.Y. Acad. Sci. 569: 86-103, 1989; Flexner et al., Vaccine 8: 17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6: 616-627, 1988; Rosenfeld et al., Science 252: 431-434, 1991; Kolls et al., Proc. Natl. Acad. Sci. USA 91: 215-219, 1994; Kass-Eisler et al., Proc. Natl. Acad. Sci. USA 90: 11498-11502, 1993; Guzman et al., Circulation 88: 2838-2848, 1993; and Guzman et al., Cir. Res. 73: 1202-1207, 1993.

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In another embodiment of the invention, a polynucleotide is administered/delivered as “naked” DNA, for example as described in Ulmer et al., Science 259: 1745-1749, 1993 and reviewed by Cohen, Science 259: 1691-1692, 1993. The uptake of naked DNA may be increased by coating the DNA onto biodegradable beads, which are efficiently transported into the cells.

In still another embodiment, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

According to another embodiment, the pharmaceutical compositions described herein will comprise one or more immunostimulants in addition to the immunogenic polynucleotide, polypeptide, antibody, T-cell, TCR, and/or APC compositions of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition is preferably one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7: 145-173, 1989.

Certain preferred adjuvants for eliciting a predominantly Th 1-type response include, for example, a combination of monophosphoryl lipid A, preferably 3-de-O-acylated monophosphoryl lipid A, together with an aluminum salt. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273: 352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other preferred formulations include more than one saponin in the adjuvant combinations of the present invention, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs. Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol to increase viscosity, or may be formulated in a dry powder form with a powder excipient such as lactose.

In one preferred embodiment, the adjuvant system includes the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other preferred formulations comprise an oil-in-water emulsion and tocopherol. Another particularly preferred adjuvant formulation employing QS21, 3D-MPL® adjuvant and tocopherol in an oil-in-water emulsion is described in WO 95/17210.

Another enhanced adjuvant system involves the combination of a CpG-containing oligonucleotide and a saponin derivative particularly the combination of CpG and QS21 is disclosed in WO 00/09159. Preferably the formulation additionally comprises an oil in water emulsion and tocopherol.

Additional illustrative adjuvants for use in the pharmaceutical compositions of the invention include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

Other preferred adjuvants include adjuvant molecules of the general formula
HO(CH₂CH₂O)ₙ-A-R, (I)
wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392: 245-251, 1998) and have been shown to be effective as a physiological adjuvant for eliciting prophylactic or therapeutic antitumor immunity (see Timmerman and Levy, Ann. Rev. Med. 50: 507-529, 1999). In general, dendritic cells may be identified based on their typical shape (stellate in situ, with marked cytoplasmic processes (dendrites) visible in vitro), their ability to take up, process and present antigens with high efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4: 594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide of the invention (or portion or other variant thereof) such that the encoded polypeptide, or an immunogenic portion thereof, is expressed on the cell surface. Such transfection may take place ex vivo, and a pharmaceutical composition comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75: 456-460, 1997. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

While any suitable carrier known to those of ordinary skill in the art may be employed in the pharmaceutical compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration.

Carriers for use within such pharmaceutical compositions are biocompatible, and may also be biodegradable. In certain embodiments, the formulation preferably provides a relatively constant level of active component release. In other embodiments, however, a more rapid rate of release immediately upon administration may be desired. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

In another illustrative embodiment, biodegradable microspheres (e.g., polylactate polyglycolate) are employed as carriers for the compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344, 5,407,609 and 5,942,252. Modified hepatitis B core protein carrier systems. such as described in WO/99 40934, and references cited therein, will also be useful for many applications. Another illustrative carrier/delivery system employs a carrier comprising particulate-protein complexes, such as those described in U.S. Pat. No. 5,928,647, which are capable of inducing a class I-restricted cytotoxic T lymphocyte responses in a host.

In another illustrative embodiment, calcium phosphate core particles are employed as carriers, vaccine adjuvants, or as controlled release matrices for the compositions of this invention. Exemplary calcium phosphate particles are disclosed, for example, in published patent application No. WO/0046147.

The pharmaceutical compositions of the invention will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles. Alternatively, a pharmaceutical composition may be stored in a freeze-dried condition requiring only the addition of a sterile liquid carrier immediately prior to use.

The development of suitable dosing and treatment regimens for using the particular compositions described herein in a variety of treatment regimens, including e.g., oral, parenteral, intravenous, intranasal, and intramuscular administration and formulation, is well known in the art, some of which are briefly discussed below for general purposes of illustration.

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (see, for example, Mathiowitz et al., Nature 1997 Mar. 27; 386(6623): 410-4; Hwang et al., Crit Rev Ther Drug Carrier Syst 1998; 15(3): 243-84; U.S. Pat. No. 5,641,515; U.S. Pat. No. 5,580,579 and U.S. Pat. No. 5,792,451). Tablets, troches, pills, capsules and the like may also contain any of a variety of additional components, for example, a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Of course, any material used in preparing any dosage unit form should be pharmaceutically pure and substantially non-toxic in the amounts employed. In addition, the active compounds may be incorporated into sustained-release preparation and formulations.

Typically, these formulations will contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al., J Controlled Release 1998 Mar. 2; 52(1-2): 81-7) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In certain embodiments, liposomes, nanocapsules, microparticles, lipid particles, vesicles, and the like, are used for the introduction of the compositions of the present invention into suitable host cells/organisms. In particular, the compositions of the present invention may be formulated for delivery either encapsulated in a lipid particle, a liposome, a vesicle, a nanosphere, or a nanoparticle or the like. Alternatively, compositions of the present invention can be bound, either covalently or non-covalently, to the surface of such carrier vehicles.

The formation and use of liposome and liposome-like preparations as potential drug carriers is generally known to those of skill in the art (see for example, Lasic, Trends Biotechnol 1998 July; 16(7): 307-21; Takakura, Nippon Rinsho 1998 March; 56(3): 691-5; Chandran et al., Indian J Exp Biol. 1997 August; 35(8): 801-9; Margalit, Crit Rev Ther Drug Carrier Syst. 1995; 12(2-3): 233-61; U.S. Pat. No. 5,567,434; U.S. Pat. No. 5,552,157; U.S. Pat. No. 5,565,213; U.S. Pat. No. 5,738,868 and U.S. Pat. No. 5,795,587, each specifically incorporated herein by reference in its entirety).

Liposomes have been used successfully with a number of cell types that are normally difficult to transfect by other procedures, including T cell suspensions, primary hepatocyte cultures and PC 12 cells (Renneisen et al., J Biol. Chem. 1990 Sep. 25; 265(27): 16337-42; Muller et al., DNA Cell Biol. 1990 April; 9(3): 221-9). In addition, liposomes are free of the DNA length constraints that are typical of viral-based delivery systems. Liposomes have been used effectively to introduce genes, various drugs, radiotherapeutic agents, enzymes, viruses, transcription factors, allosteric effectors and the like, into a variety of cultured cell lines and animals. Furthermore, he use of liposomes does not appear to be associated with autoimmune responses or unacceptable toxicity after systemic delivery.

In certain embodiments, liposomes are formed from phospholipids that are dispersed in an aqueous medium and spontaneously form multilamellar concentric bilayer vesicles (also termed multilamellar vesicles (MLVs).

Alternatively, in other embodiments, the invention provides for pharmaceutically-acceptable nanocapsule formulations of the compositions of the present invention. Nanocapsules can generally entrap compounds in a stable and reproducible way (see, for example, Quintanar-Guerrero et al., Drug Dev Ind Pharm. 1998 December; 24(12): 1113-28). To avoid side effects due to intracellular polymeric overloading, such ultrafine particles (sized around 0.1 μm) may be designed using polymers able to be degraded in vivo. Such particles can be made as described, for example, by Couvreur et al., Crit Rev Ther Drug Carrier Syst. 1988; 5(1): 1-20; zur Muhlen et al., Eur J Pharm Biopharm. 1998 March; 45(2): 149-55; Zambaux et al. J Controlled Release. 1998 Jan. 2; 50(1-3): 31-40; and U.S. Pat. No. 5,145,684.

Cancer Therapeutic Methods

Immunologic approaches to cancer therapy are based on the recognition that cancer cells can often evade the body's defenses against aberrant or foreign cells and molecules, and that these defenses might be therapeutically stimulated to regain the lost ground, e.g. pgs. 623-648 in Klein, Immunology (Wiley-Interscience, New York, 1982). Numerous recent observations that various immune effectors can directly or indirectly inhibit growth of tumors has led to renewed interest in this approach to cancer therapy, e.g. Jager, et al., Oncology 2001; 60(1): 1-7; Renner, et al., Ann Hematol 2000 December; 79(12): 651-9.

Four-basic cell types whose function has been associated with antitumor cell immunity and the elimination of tumor cells from the body are: i) B-lymphocytes which secrete immunoglobulins into the blood plasma for identifying and labeling the nonself invader cells; ii) monocytes which secrete the complement proteins that are responsible for lysing and processing the immunoglobulin-coated target invader cells; iii) natural killer lymphocytes having two mechanisms for the destruction of tumor cells, antibody-dependent cellular cytotoxicity and natural killing; and iv) T-lymphocytes possessing antigen-specific receptors and having the capacity to recognize a tumor cell carrying complementary marker molecules (Schreiber, H., 1989, in Fundamental Immunology (ed). W. E. Paul, pp. 923-955).

Cancer immunotherapy generally focuses on inducing humoral immune responses, cellular immune responses, or both. Moreover, it is well established that induction of CD4⁺ T helper cells is necessary in order to secondarily induce either antibodies or cytotoxic CD8⁺ T cells. Polypeptide antigens that are selective or ideally specific for cancer cells, particularly colon cancer cells, offer a powerful approach for inducing immune responses against colon cancer, and are an important aspect of the present invention.

Therefore, in further aspects of the present invention, the pharmaceutical compositions described herein may be used to stimulate an immune response against cancer, particularly for the immunotherapy of colon cancer. Within such methods, the pharmaceutical compositions described herein are administered to a patient, typically a warm-blooded animal, preferably a human. A patient may or may not be afflicted with cancer. Pharmaceutical compositions and vaccines may be administered either prior to or following surgical removal of primary tumors and/or treatment such as administration of radiotherapy or conventional chemotherapeutic drugs. As discussed above, administration of the pharmaceutical compositions may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Monoclonal antibodies may be labeled with any of a variety of labels for desired selective usages in detection, diagnostic assays or therapeutic applications (as described in U.S. Pat. Nos. 6,090,365; 6,015,542; 5,843,398; 5,595,721; and 4,708,930, hereby incorporated by reference in their entirety as if each was incorporated individually). In each case, the binding of the labelled monoclonal antibody to the determinant site of the antigen will signal detection or delivery of a particular therapeutic agent to the antigenic determinant on the non-normal cell. A further object of this invention is to provide the specific monoclonal antibody suitably labelled for achieving such desired selective usages thereof.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157: 177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to a tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnostic Compositions, Methods and Kits

In general, a cancer may be detected in a patient based on the presence of one or more colon tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as colon cancer. In addition, such proteins may be useful for the detection of other cancers. The binding agents provided herein generally permit detection of the level of antigen that binds to the agent in the biological sample.

Polynucleotide primers and probes may be used to detect the level of mRNA encoding a tumor protein, which is also indicative of the presence or absence of a cancer. In general, a tumor sequence should be present at a level that is at least two-fold, preferably three-fold, and more preferably five-fold or higher in tumor tissue than in normal tissue of the same type from which the tumor arose. Expression levels of a particular tumor sequence in tissue types different from that in which the tumor arose are irrelevant in certain diagnostic embodiments since the presence of tumor cells can be confirmed by observation of predetermined differential expression levels, e.g., 2-fold, 5-fold, etc, in tumor tissue to expression levels in normal tissue of the same type.

Other differential expression patterns can be utilized advantageously for diagnostic purposes. For example, in one aspect of the invention, overexpression of a tumor sequence in tumor tissue and normal tissue of the same type, but not in other normal tissue types, e.g. PBMCs, can be exploited diagnostically. In this case, the presence of metastatic tumor cells, for example in a sample taken from the circulation or some other tissue site different from that in which the tumor arose, can be identified and/or confirmed by detecting expression of the tumor sequence in the sample, for example using RT-PCR analysis. In many instances, it will be desired to enrich for tumor cells in the sample of interest, e.g., PBMCs, using cell capture or other like techniques.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length colon tumor proteins and polypeptide portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with colon cancer at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as colon cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 g, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use tumor polypeptides to detect antibodies that bind to such polypeptides in a biological sample. The detection of such tumor protein specific antibodies may correlate with the presence of a cancer.

A cancer may also, or alternatively, be detected based on the presence of T cells that specifically react with a tumor protein in a biological sample. Within certain methods, a biological sample comprising CD4⁺ and/or CD8⁺ T cells isolated from a patient is incubated with a tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding a tumor protein in a biological sample. For example, at least two oligonucleotide primers may be employed in a polymerase chain reaction (PCR) based assay to amplify a portion of a tumor cDNA derived from a biological sample, wherein at least one of the oligonucleotide primers is specific for (i.e., hybridizes to) a polynucleotide encoding the tumor protein. The amplified cDNA is then separated and detected using techniques well known in the art, such as gel electrophoresis.

Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a tumor protein of the invention that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, for example, Mullis et al., Cold Spring Harbor Symp. Quant. Biol., 51: 263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another aspect of the present invention, cell capture technologies may be used in conjunction, with, for example, real-time PCR to provide a more sensitive tool for detection of metastatic cells expressing colon tumor antigens. Detection of colon cancer cells in biological samples, e.g., bone marrow samples, peripheral blood, and small needle aspiration samples is desirable for diagnosis and prognosis in colon cancer patients.

Immunomagnetic beads coated with specific monoclonal antibodies to surface cell markers, or tetrameric antibody complexes, may be used to first enrich or positively select cancer cells in a sample. Various commercially available kits may be used, including Dynabeads® Epithelial Enrich (Dynal Biotech, Oslo, Norway), StemSep™ (StemCell Technologies, Inc., Vancouver, BC), and RosetteSep (StemCell Technologies). A skilled artisan will recognize that other methodologies and kits may also be used to enrich or positively select desired cell populations. Dynabeads® Epithelial Enrich contains magnetic beads coated with mAbs specific for two glycoprotein membrane antigens expressed on normal and neoplastic epithelial tissues. The coated beads may be added to a sample and the sample then applied to a magnet, thereby capturing the cells bound to the beads. The unwanted cells are washed away and the magnetically isolated cells eluted from the beads and used in further analyses.

RosetteSep can be used to enrich cells directly from a blood sample and consists of a cocktail of tetrameric antibodies that targets a variety of unwanted cells and crosslinks them to glycophorin A on red blood cells (RBC) present in the sample, forming rosettes. When centrifuged over Ficoll, targeted cells pellet along with the free RBC. The combination of antibodies in the depletion cocktail determines which cells will be removed and consequently which cells will be recovered. Antibodies that are available include, but are not limited to: CD2, CD3, CD4, CD5, CD8, CD10, CD11b, CD14, CD15, CD16, CD19, CD20, CD24, CD25, CD29, CD33, CD34, CD36, CD38, CD41, CD45, CD45RA, CD45RO, CD56, CD66B, CD66e, HLA-DR, IgE, and TCRαβ.

Additionally, it is contemplated in the present invention that mAbs specific for colon tumor antigens can be generated and used in a similar manner. For example, mAbs that bind to tumor-specific cell surface antigens may be conjugated to magnetic beads, or formulated in a tetrameric antibody complex, and used to enrich or positively select metastatic colon tumor cells from a sample. Once a sample is enriched or positively selected, cells may be lysed and RNA isolated. RNA may then be subjected to RT-PCR analysis using colon tumor-specific primers in a real-time PCR assay as described herein. One skilled in the art will recognize that enriched or selected populations of cells may be analyzed by other methods (e.g. in situ hybridization or flow cytometry).

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to a tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding a tumor protein in a biological sample. Such kits generally comprise at least one oligonucleotide probe or primer, as described above, that hybridizes to a polynucleotide encoding a tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding a tumor protein.

The following Examples are offered by way of illustration and not by way of limitation.

### EXAMPLES

### Example 1

### Preparation of Colon Tumor Subtraction Libraries and Identification of Colon Tumor Protein cDNAs

This Example illustrates the identification of cDNA molecules encoding colon tumor proteins. PolyA mRNA was prepared from a pool of three colon tumor cell lines (adenocarcinomas) grown in SCID mice were subtracted with a set of transcripts from normal lung, adrenal gland, bone marrow, small intestine, stomach, pancreas, normal colon, HMEC (human mammary epithelial cell line) and SCID mouse liver/spleen samples. The cDNA synthesis, hybridizations, and PCR amplifications were performed according to standard procedures (Clontech), with modifications at the cDNA digestion steps and in the tester to driver hybridization ratios. Following the PCR amplification steps, the cDNAs were cloned into the pCR2.1 plasmid vector. To analyze the efficiency of the subtraction, the housekeeping gene, actin, was PCR amplified from dilutions of subtracted as well as unsubtracted PCR samples. This results suggest that the library was enriched for genes overexpressed in colon tumor samples.

The Clontech PCR-based cDNA subtraction approach was utilized to prepare two cDNA libraries from pools of tester mRNA collected from three Dukes B stage colon tumor samples. Eight normal tissues, including lung, adrenal gland, bone marrow, small intestine, heart, pancreas, colon, and liver were represented in the driver mRNA pool. The two libraries, CS/B 1105 and CS/B 1605, shared the same tester and driver mRNA samples but differed in their tester:driver ratios (1:5 and 1:30, respectively). To analyze the efficiency of the subtraction, the housekeeping gene, actin, was PCR amplified from dilutions of subtracted as well as unsubtracted PCR samples. This results suggest that the library was enriched for genes overexpressed in colon tumor samples. 172 randomly selected clones were subjected to DNA sequencing and are presented herein as SEQ ID NO: 57-229. Additional sequence data was generated by bulk sequencing clones isolated from the CS/B1105 and CS/B11605 subtraction libraries and are presented herein as SEQ ID NO: 230-1660.

Further disclosed herein are sequences derived from a fourth colon tumor expression library which sequences are presented herein as SEQ ID NO: 1661-1704.

Antigens obtained from this colon PCR subtracted cDNA libraries may be used for immunotherapeutic purposes in individuals with colon adenocarcinoma and/or as diagnostic markers for colon adenocarcinoma.

### Example 2

### Analysis of cDNA Expression Using Microarray Technology

In additional studies, sequences disclosed herein were evaluated for overexpression in specific tumor tissues by microarray analysis. Using this approach, cDNA sequences were PCR amplified and their mRNA expression profiles in tumor and normal tissues were examined using cDNA microarray technology essentially as described (Schena et al., Science 270(5235): 467-70 (1995). In brief, the clones were arrayed onto glass slides as multiple replicas, with each location corresponding to a unique cDNA clone (as many as 5500 clones can be arrayed on a single slide, or chip). Each chip was hybridized with a pair of cDNA probes that were fluorescence-labeled with Cy3 and Cy5, respectively. Typically, 1 μg of polyA⁺ RNA was used to generate each cDNA probe. After hybridization, the chips were scanned and the fluorescence intensity recorded for both Cy3 and Cy5 channels. There were multiple built-in quality control steps. First, the probe quality was monitored using a panel of ubiquitously expressed genes. Secondly, the control plate also includee yeast DNA fragments of which complementary RNA were spiked into the probe synthesis for measuring the quality of the probe and the sensitivity of the analysis. Currently, this methodology offers a sensitivity of 1 in 100,000 copies of mRNA. Finally, the reproducibility of this technology was ensured by including duplicated control cDNA elements at different locations.

Table 2 identifies 27 clones found to be at least two-fold overexpressed in colon tumor cells as compared to a panel of normal tissues by microarray analysis.

TABLE 2

| array | Clone Sequence Identifier | Ratio | clone I.D. |
|---|---|---|---|
| p0175r03c18 | R0676 F9 | 2.62 | 72239, |
| p0174r13c21 | R0675 A11 | 2.16 | 72237, |
| p0174r09c13 | R0674 A7 | 2.67 | 72236, |
| p0176r01c22 | R0680 B11 | 2.3 | 72244, |
| p0174r05c17 | R0673 A9 | 2.09 | 72234, |
| p0174r08c24 | R0673 H12 | 2.06 | 71574, 72235 |
| p0174r16c17 | R0675 G9 | 2.46 | 72238, |
| p0175r07c22 | R0677 F11 | 3.21 | 72241, |
| p0176r03c02 | R0680 F1 | 2.93 | 72245, |
| p0176r04c06 | R0680 H3 | 2.09 | 72246, |
| p0177r07c22 | R0685 F11 | 2.27 | 71675, 72247, |
| | | | 72902, 71041 |
| p0177r13c06 | R0687 B3 | 3.43 | 72249, 72904, 70985 |
| p0175r10c04 | R0678 D2 | 2.05 | 70424, 72899 |
| p0176r16c05 | R0683 G3 | 2.03 | 70426, 72900 |
| p0174r07c23 | R0673 E12 | 2.58 | 72901, |
| p0174r03c05 | R0672 E3 | 2.09 | 72233 |
| p0175r06c13 | R0677 C7 | 2.13 | 72240 |
| p0175r11c19 | R0678 E10 | 3.44 | 72242 |
| p0175r14c21 | R0679 C11 | 2.75 | 72243 |
| p0174r10c20 | R0674 D10 | 2.58 | 71575 |
| p0172r01c06 | R0664 B3 | 2.05 | 71569 |
| p0173r09c05 | R0670 A3 | 2.35 | 71571 |
| p0172r05c18 | R0665 B9 | 2.36 | 70580 |
| p0175r04c07 | 676_G4 & 678_H12 & | 3.94 | 70581, 70582, |
| | 681_B5 & 682_E4 | | 70586, 70589 |
| p0176r07c14 | R0681 F7 | 2.27 | 70587 |
| p0176r08c22 | R0681 H11 | 2.02 | 70584 |
| p0176r08c06 | R0681 H3 | 2.25 | 70588 |

In addition, the following clones (Table 3) were repeatedly identified by microarray analysis as being at least two-fold overexpressed in colon tumor cells as compared to a panel of normal tissues.

TABLE 3

| | | | |
|---|---|---|---|
| 70971 | 70973 | 70974 | 71049 |
| 70975 | 70977 | 70980 | 71058 |
| 70981 | 70982 | 70986 | 71063 |
| 70987 | 70988 | 70997 | 71051 |
| 70998 | 70999 | 71006 | 71059 |
| 71008 | 71009 | 71011 | 71065 |
| 71012 | 71018 | 71021 | 71055 |
| 71022 | 71024 | 71028 | 71062 |
| 71029 | 71032 | 71036 | 71066 |
| 71037 | 71039 | 71045 | |

### Example 3

### Analysis of cDNA Expression Using Real-Time PCR

Two clones isolated from the subtraction library described in Example 1 and that showed at least 2-fold overexpression in colon tumors by microarray, were selected for further mRNA expression analysis by real-time PCR. The first clone, C1490P (SEQ ID NO:1660; also referred to as clone R0680 B11 and 72244), showed no significant similarity to any known sequences when searched against the Genbank nucleic acid database. The second clone, C1491P (SEQ ID NO:1681; also referred to as clone R0683 G3 and 70426), has some similarity to adenovirus EIA enhancer binding protein (set forth in SEQ ID NO:1788 (cDNA) and 1789 (amino acid)).

The first-strand cDNA used in the quantitative real-time PCR was synthesized from 20 μg of total RNA that was treated with DNase I (Amplification Grade, Gibco BRL Life Technology, Gaithersburg, Md.), using Superscript Reverse Transcriptase (RT) (Gibco BRL Life Technology, Gaithersburg, Md.). Real-time PCR was performed with a GeneAmp™ 5700 sequence detection system (PE Biosystems, Foster City, Calif.). The 5700 system uses SYBR™ green, a fluorescent dye that only intercalates into double stranded DNA, and a set of gene-specific forward and reverse primers. The increase in fluorescence was monitored during the whole amplification process. The optimal concentration of primers was determined using a checkerboard approach and a pool of cDNAs from breast tumors was used in this process. The PCR reaction was performed in 25 μl volumes that include 2.5 μl of SYBR green buffer, 2 μl of cDNA template and 2.5 μl each of the forward and reverse primers for the gene of interest. The cDNAs used for RT reactions were diluted 1:10 for each gene of interest and 1:100 for the β-actin control. In order to quantitate the amount of specific cDNA (and hence initial mRNA) in the sample, a standard curve was generated for each run using the plasmid DNA containing the gene of interest. Standard curves were generated using the Ct values determined in the real-time PCR which were related to the initial cDNA concentration used in the assay. Standard dilution ranging from 20-2×10⁶ copies of the gene of interest was used for this purpose. In addition, a standard curve was generated for β-actin ranging from 200 fg-2000 fg. This enabled standardization of the initial RNA content of a tissue sample to the amount of β-actin for comparison purposes. The mean copy number for each group of tissues tested was normalized to a constant amount of β-actin, allowing the evaluation of the over-expression levels seen with each of the genes.

The real-time analysis confirmed previous microarray results and showed that C1490P is overexpressed in the majority of colon tumor samples in comparison to normal samples. Overexpression of C1490P was also seen in lymph nodes and thymus. Some C1490P expression was observed in normal colon but at a much lower level than was seen in tumor samples. Likewise, some low levels of expression were observed in breast, esophagus, small intestine, stomach, trachea, thymus, and bone marrow. C1491P is overexpressed in the majority of colon tumor samples when compared to normal colon and a panel of other normal tissue. Low expression of this gene was observed in normal pancreas, pituitary, and low expression in some salivary and adrenal gland samples. Thus, the results indicate that these 2 candidates may be used for immunotherapeutic purposes in individuals with colon cancer and/or as diagnostic markers for colon cancer.

### Example 4

### Peptide Priming of T-Helper Lines

Generation of CD4⁺ T helper lines and identification of peptide epitopes derived from tumor-specific antigens that are capable of being recognized by CD4⁺ T cells in the context of HLA class II molecules, is carried out as follows:

Fifteen-mer peptides overlapping by 10 amino acids, derived from a tumor-specific antigen, are generated using standard procedures. Dendritic cells (DC) are derived from PBMC of a normal donor using GM-CSF and IL-4 by standard protocols. CD4⁺ T cells are generated from the same donor as the DC using MACS beads (Miltenyi Biotec, Auburn, Calif.) and negative selection. DC are pulsed overnight with pools of the 15-mer peptides, with each peptide at a final concentration of 0.25 μg/ml. Pulsed DC are washed and plated at 1×10⁴ cells/well of 96-well V-bottom plates and purified CD4⁺ T cells are added at 1×10⁵/well. Cultures are supplemented with 60 ng/ml IL-6 and 10 ng/ml IL-12 and incubated at 37° C. Cultures are restimulated as above on a weekly basis using DC generated and pulsed as above as antigen presenting cells, supplemented with 5 ng/ml IL-7 and 10 U/ml IL-2. Following 4 in vitro stimulation cycles, resulting CD4⁺ T cell lines (each line corresponding to one well) are tested for specific proliferation and cytokine production in response to the stimulating pools of peptide with an irrelevant pool of peptides used as a control.

### Example 5

### Generation of Tumor-Specific CTL Lines Using In Vitro Whole-Gene Priming

Using in vitro whole-gene priming with tumor antigen-vaccinia infected DC (see, for example, Yee et al, The Journal of Immunology, 157(9): 4079-86, 1996), human CTL lines are derived that specifically recognize autologous fibroblasts transduced with a specific tumor antigen, as determined by interferon-γ ELISPOT analysis. Specifically, dendritic cells (DC) are differentiated from monocyte cultures derived from PBMC of normal human donors by growing for five days in RPMI medium containing 10% human serum, 50 ng/ml human GM-CSF and 30 ng/ml human IL-4. Following culture, DC are infected overnight with tumor antigen-recombinant vaccinia virus at a multiplicity of infection (M.O.I) of five, and matured overnight by the addition of 3 μg/ml CD40 ligand. Virus is then inactivated by UV irradiation. CD8+ T cells are isolated using a magnetic bead system, and priming cultures are initiated using standard culture techniques. Cultures are restimulated every 7-10 days using autologous primary fibroblasts retrovirally transduced with previously identified tumor antigens. Following four stimulation cycles, CD8+ T cell lines are identified that specifically produce interferon-γ when stimulated with tumor antigen-transduced autologous fibroblasts. Using a panel of HLA-mismatched B-LCL lines transduced with a vector expressing a tumor antigen, and measuring interferon-γ production by the CTL lines in an ELISPOT assay, the HLA restriction of the CTL lines is determined.

### Example 6

### Generation and Characterization of Anti-Tumor Antigen Monoclonal Antibodies

Mouse monoclonal antibodies are raised against E. Coli derived tumor antigen proteins as follows: Mice are immunized with Complete Freund's Adjuvant (CFA) containing 50 μg recombinant tumor protein, followed by a subsequent intraperitoneal boost with Incomplete Freund's Adjuvant (IFA) containing 10 μg recombinant protein. Three days prior to removal of the spleens, the mice are immunized intravenously with approximately 50 μg of soluble recombinant protein. The spleen of a mouse with a positive titer to the tumor antigen is removed, and a single-cell suspension made and used for fusion to SP2/O myeloma cells to generate B cell hybridomas. The supernatants from the hybrid clones are tested by ELISA for specificity to recombinant tumor protein, and epitope mapped using peptides that spanned the entire tumor protein sequence. The mAbs are also tested by flow cytometry for their ability to detect tumor protein on the surface of cells stably transfected with the cDNA encoding the tumor protein.

### Example 7

### Synthesis of Polypeptides

Polypeptides are synthesized on a Perkin Elmer/Applied Biosystems Division 430A peptide synthesizer using FMOC chemistry with HPTU (O-Benzotriazole-N,N,N′,N′-tetramethyluronium hexafluorophosphate) activation. A Gly-Cys-Gly sequence is attached to the amino terminus of the peptide to provide a method of conjugation, binding to an immobilized surface, or labeling of the peptide. Cleavage of the peptides from the solid support is carried out using the following cleavage mixture: trifluoroacetic acid:ethanedithiol:thioanisole:water:phenol (40:1:2:2:3). After cleaving for 2 hours, the peptides are precipitated in cold methyl-t-butyl-ether. The peptide pellets are then dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) is used to elute the peptides. Following lyophilization of the pure fractions, the peptides are characterized using electrospray or other types of mass spectrometry and by amino acid analysis.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An isolated polynucleotide comprising a sequence selected from the group consisting of:
(a) sequences provided in SEQ ID NO:1-1788;
(b) complements of the sequences provided in SEQ ID NO:1-1788;
(c) sequences consisting of at least 20 contiguous residues of a sequence provided in SEQ ID NO:1-1788;
(d) sequences that hybridize to a sequence provided in SEQ ID NO:1-1788, under moderately stringent conditions;
(e) sequences having at least 75% identity to a sequence of SEQ ID NO:1-1788;
(f) sequences having at least 90% identity to a sequence of SEQ ID NO:1-1788; and
(g) degenerate variants of a sequence provided in SEQ ID NO:1-1788.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1;
(c) sequences having at least 90% identity to a sequence encoded by a polynucleotide of claim 1;
(d) sequences set forth in SEQ ID NO:1789;
(e) sequences having at least 70% identity to a sequence set forth in SEQ ID NO:1789; and
(f) sequences having at least 90% identity to a sequence set forth in SEQ ID NO:1789.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NO:1-1788 under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polypeptide according to claim 2,
under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells,
and thereby inhibiting the development of a cancer in the patient.
